SEQUENCE LISTING (60 sequences)

SEQ ID NO: 1 - protein, 6 aa, Artificial Sequence
  Peptide Linker
GGGGGK

SEQ ID NO: 2 - protein, 7 aa, Artificial Sequence
  Peptide Linker
GGGGGKR

SEQ ID NO: 3 - protein, 8 aa, Artificial Sequence
  Peptide Linker
GGGKGGGG

SEQ ID NO: 4 - protein, 8 aa, Artificial Sequence
  Peptide Linker
GGGNGSGG

SEQ ID NO: 5 - protein, 8 aa, Artificial Sequence
  Peptide Linker
GGGCGGGG

SEQ ID NO: 6 - protein, 5 aa, Artificial Sequence
  Peptide Linker
GPNGG

SEQ ID NO: 7 - protein, 6 aa, Artificial Sequence
  Peptide Linker
GGEGGG

SEQ ID NO: 8 - protein, 8 aa, Artificial Sequence
  Peptide Linker
GGEEEGGG

SEQ ID NO: 9 - protein, 5 aa, Artificial Sequence
  Peptide Linker
GEEEG

SEQ ID NO: 10 - protein, 4 aa, Artificial Sequence
  Peptide Linker
GEEE

SEQ ID NO: 11 - protein, 6 aa, Artificial Sequence
  Peptide Linker
GGDGGG

SEQ ID NO: 12 - protein, 7 aa, Artificial Sequence
  Peptide Linker
GGDDDGG

SEQ ID NO: 13 - protein, 5 aa, Artificial Sequence
  Peptide Linker
GDDDG

SEQ ID NO: 14 - protein, 4 aa, Artificial Sequence
  Peptide Linker
GDDD

SEQ ID NO: 15 - protein, 21 aa, Artificial Sequence
  Peptide Linker
GGGGSDDSDEGSDGEDGGGGS

SEQ ID NO: 16 - protein, 5 aa, Artificial Sequence
  Peptide Linker
WEWEW

SEQ ID NO: 17 - protein, 5 aa, Artificial Sequence
  Peptide Linker
FEFEF

SEQ ID NO: 18 - protein, 6 aa, Artificial Sequence
  Peptide Linker
EEEWWW

SEQ ID NO: 19 - protein, 6 aa, Artificial Sequence
  Peptide Linker
EEEFFF

SEQ ID NO: 20 - protein, 7 aa, Artificial Sequence
  Peptide Linker
WWEEEWW

SEQ ID NO: 21 - protein, 7 aa, Artificial Sequence
  Peptide Linker
FFEEEFF

SEQ ID NO: 22 - protein, 3 aa, Artificial Sequence
  Peptide Linker
GGG

SEQ ID NO: 23 - protein, 4 aa, Artificial Sequence
  Peptide Linker
GGGG

SEQ ID NO: 24 - protein, 5 aa, Artificial Sequence
  Peptide Linker
GGGGG

SEQ ID NO: 25 - protein, 6 aa, Artificial Sequence
  Peptide Linker
GGGGGG

SEQ ID NO: 26 - protein, 7 aa, Artificial Sequence
  Peptide Linker
GGGGGGG

SEQ ID NO: 27 - protein, 5 aa, Artificial Sequence
  Peptide Linker
GGGGS

SEQ ID NO: 28 - protein, 9 aa, Artificial Sequence
  Peptide Linker
GGGSGGGGS

SEQ ID NO: 29 - protein, 10 aa, Artificial Sequence
  Peptide Linker
GGGGSGGGGS

SEQ ID NO: 30 - protein, 2 aa, Artificial Sequence
  Peptide Linker
GG

SEQ ID NO: 31 - protein, 15 aa, Artificial Sequence
  Peptide Linker
GGGGSGGGGSGGGGS

SEQ ID NO: 32 - protein, 25 aa, Artificial Sequence
  Peptide Linker
GGGGSGGGGSGGGGSGGGGSGGGGS

SEQ ID NO: 33 - protein, 4 aa, Artificial Sequence
  Peptide Linker
GAGA

SEQ ID NO: 34 - protein, 6 aa, Artificial Sequence
  Peptide Linker
GAGAGA

SEQ ID NO: 35 - protein, 8 aa, Artificial Sequence
  Peptide Linker
GAGAGAGA

SEQ ID NO: 36 - protein, 2 aa, Artificial Sequence
  Peptide Linker
AA

SEQ ID NO: 37 - protein, 3 aa, Artificial Sequence
  Peptide Linker
AAA

SEQ ID NO: 38 - protein, 4 aa, Artificial Sequence
  Peptide Linker
AAAA

SEQ ID NO: 39 - protein, 5 aa, Artificial Sequence
  Peptide Linker
AAAAA

SEQ ID NO: 40 - protein, 6 aa, Artificial Sequence
  Peptide Linker
AAAAAA

SEQ ID NO: 41 - protein, 7 aa, Artificial Sequence
  Peptide Linker
AAAAAAA

SEQ ID NO: 42 - protein, 8 aa, Artificial Sequence
  Peptide Linker
AAAAAAAA

SEQ ID NO: 43 - protein, 8 aa, Artificial Sequence
  Peptide Linker
GGGSGGGS

SEQ ID NO: 44 - protein, 12 aa, Artificial Sequence
  Peptide Linker
GGGSGGGSGGGS

SEQ ID NO: 45 - protein, 16 aa, Artificial Sequence
  Peptide Linker
GGGSGGGSGGGSGGGS

SEQ ID NO: 46 - protein, 20 aa, Artificial Sequence
  Peptide Linker
GGGSGGGSGGGSGGGSGGGS

SEQ ID NO: 47 - protein, 24 aa, Artificial Sequence
  Peptide Linker
GGGSGGGSGGGSGGGSGGGSGGGS

SEQ ID NO: 48 - protein, 5 aa, Artificial Sequence
  Peptide Linker
GGGGS

SEQ ID NO: 49 - protein, 20 aa, Artificial Sequence
  Peptide Linker
GGGGSGGGGSGGGGSGGGGS

SEQ ID NO: 50 - protein, 30 aa, Artificial Sequence
  Peptide Linker
GGGGSGGGGSGGGGSGGGGSGGGGSGGGGS

SEQ ID NO: 51 - protein, 8 aa, Artificial Sequence
  Peptide Linker
GGGGGGGG

SEQ ID NO: 52 - protein, 22 aa, Artificial Sequence
  Signal Peptide
MDMRVPAQLLGLLLLWLRGARC

SEQ ID NO: 53 - protein, 19 aa, Artificial Sequence
  Signal Peptide
MAWALLLLTLLTQGTGSWA

SEQ ID NO: 54 - protein, 19 aa, Artificial Sequence
  Signal Peptide
MTCSPLLLTLLIHCTGSWA

SEQ ID NO: 55 - protein, 20 aa, Artificial Sequence
  Signal Peptide
MEAPAQLLFLLLLWLPDTTG

SEQ ID NO: 56 - protein, 19 aa, Artificial Sequence
  Signal Peptide
MEWTWRVLFLVAAATGAHS

SEQ ID NO: 57 - protein, 20 aa, Artificial Sequence
  Signal Peptide
METPAQLLFLLLLWLPDTTG

SEQ ID NO: 58 - protein, 20 aa, Artificial Sequence
  Signal Peptide
METPAQLLFLLLLWLPDTTG

SEQ ID NO: 59 - protein, 19 aa, Artificial Sequence
  Signal Peptide
MKHLWFFLLLVAAPRWVLS

SEQ ID NO: 60 - protein, 19 aa, Artificial Sequence
  Signal Peptide
MEWSWVFLFFLSVTTGVHS

(19) United States
(12) Patent Application Publication
(10) Pub. No.: US 2023/0374162 A1
(43) Pub. Date: Nov. 23, 2023

(54) RATIONAL SELECTION OF BUILDING BLOCKS FOR THE ASSEMBLY OF MULTISPECIFIC ANTIBODIES

(71) Applicant: AMGEN INC., Thousand Oaks, CA (US)

(72) Inventors: Danyang GONG, Arcadia, CA (US); Fernando GARCES, San Francisco, CA (US); Zhulun WANG, Los Altos, CA (US)

(73) Assignee: AMGEN INC., Thousand Oaks, CA (US)

(21) Appl. No.: 18/248,345

(22) PCT Filed: Oct. 5, 2021
(86) PCT No.: PCT/US2021/053650

§ 371 Date: Apr. 7, 2023

Related U.S. Application Data
(60) Provisional application No. 63/088,972, filed on Oct. 7, 2020.

(51) Int. Cl. C07K 16/46; C12N 15/63

(52) U.S. Cl. CPC C07K 16/468; C12N 15/63; C07K 2317/31; C07K 2317/565; C07K 2317/55; C07K 2317/622

(57) ABSTRACT

The ability to generate a single antibody-based construct that can recognize multiple targets simultaneously, is paramount to advance many therapeutics candidates to clinic. Often, this implies extensive protein design with vary degrees of success. In the case of multispecific antibody constructs, there are multiple modalities from which to choose and often multiple antigen binders as well. Described here is the discovery of new methods to optimally pair antigen binders with the proper format, including the selection of common light chains.

### FIELD OF THE INVENTION

The present invention relates to the field of biopharmaceuticals. In particular, the invention relates to methods of making multispecific antibody constructs and methods of choosing the optimal multispecific module for such constructs.

### BACKGROUND OF THE INVENTION

Bispecific antibodies (Bispecifics) represent an exciting new generation of large molecule therapeutics in a field currently dominated by monoclonal antibodies (mAbs)^{1,2}. A defining feature of Bispecifics is the ability to recognize two epitopes located on the same or distinct targets. This dual-recognition capability expands the functionality of conventional mAbs, allowing for diverse applications such as recruiting immune cells to destroy tumor cells, crosslinking distinct cell surface receptors or enhancing tissue specificity^{1,3}. For example, Amgen's Bispecific T-cell Engager (BiTE®) binds both a CD3 epitope on the surface of T cells and a tumor-associated antigen^{4,5}, effectively acting as a bridge to link immunologically active T cells and target tumor cells. To date, over 100 bispecific formats have been reported, with over 85 in development and three receiving FDA approval^{1,6, 7}. Generally, Bispecifics can be classified in three categories: i) fragment fusion (e.g. tandem scFv and DART), ii) IgG fusion (e.g. IgG-scFv and DVD-Ig) and iii) IgG-like molecule (e.g. Hetero-Fc)^{7,8}. While fragment fusions and IgG-fusions show a simple engineered configuration (only one or two polypeptide chains), which favors purification and stable cell line generation, these formats often display low yield and undesirable stability profile. In contrast, IgG-like Bispecifics that mimic the native structure of IgG molecules (e.g. Hetero-Fc) show higher stability and superior cell production. Moreover, they are among the most represented bispecific formats in clinical trials, possibly due to the good half-life profile in serum and low potential for immunogenicity¹. However, due to the high number of chains (3-4) in these formats, mispaired species are a significant contaminant which require multiple purification steps to remove and can significantly reduce the final purification yield.

In an engineered IgG-like bispecific antibody, multiple Heavy Chains (HCs) and Light Chains (LCs) are assembled into a single molecule to enable the recognition of two distinct epitopes. Therefore, a challenge for developing IgG-like Bispecifics is to ensure the correct chain pairing. In the case of 4-chain Hetero-Fcs, the co-expression of these chains in the same cell can result in 9 possible combinations of mispaired IgG species^{9,10}. In the past few years, several strategies including knobs-into-holes¹¹, strand-exchange engineered domain¹² and charge pair mutations (CPMs)^{13,14}, have been developed to address the HC/HC and HC/LC pairing problems. In most cases of HC/LC engineering, the rationale is to engineer the chain interface in such a way that favors cognate HC/LC pairing over non-cognate. However, despite the best engineering efforts, sequence diversity (complementarity-determining regions (CDRs), framework, and LC isotype) often limits the success of these engineering tools when applied as a rigid platform to HC/LC pairing.

To overcome these difficulties, the usage of a common Light Chain (cLC) is appealing as it avoids the need to drive pairing between specific HCs and LCs. However, the identification of a cLC that maintains the desired binding profile to distinct epitopes when paired with different HCs is both challenging and often requires significant investment early in the drug development process¹⁵. In general, two methods are most commonly used to discover antibodies that carry a cLC. The first involves the screening of display libraries that consists of diverse HC sequences but only one or few LCs. Alternatively, mice expressing a universal LC are immunized for each of the desired targets^{16,17} Since both approaches restrict the available LCs, these cLC antibodies often show suboptimal binding affinities requiring extensive engineering, mostly in the HC, to optimize target affinity. In contrast, both HC and LC can be targeted for optimization efforts in regular mAbs. Moreover, structures of antibody-antigen complexes reveal that much of antibodies/epitope interactions are HC driven and in some rare cases the LC does not make any productive interactions¹⁸. Accordingly, some LCs may be suitable for pairing with non-cognate HCs while retaining target binding affinity. The cLC Hetero-Fcs assembled with such LCs, together with the cognate and non-cognate HCs, require less optimization as they retain the native affinity in their cognate HC/LC arms.

The development of therapeutic antibodies usually starts with immunization of humanized animal models with selected antigens^{19,20} leading to the identification and isolation of lead mAbs. These mAbs are selected to meet design goals such as target specificity, binding affinity, cross-species reactivity, yield, stability and no immunogenicity, among others, but little is known of the properties required for those that will become building blocks for Bispecifics. Frequently, this demands the empirical testing of hundreds of Bispecifics to evaluate every parental mAb combination, resulting in increased timelines and resources.

Accordingly, there is a need for two high-throughput screening methods to facilitate the rational selection of lead mAbs to make Bispecifics and multispecifics. The present application describes competition and non-competition Chain Selectivity Assessment (CSA). Using competition CSA (cCSA), mAbs whose HCs & LCs assemble effectively into Hetero-Fc molecules are selected with little or minimal cross-pairing between the two Fabs. Non-competition CSA (ncCSA is a powerful tool to identify cLCs in a cost-effective manner.

### SUMMARY OF THE INVENTION

In one aspect the present invention is directed to a method for selecting a multispecific antibody construct, the method comprising:
(a) obtaining a plurality of antibody Fab fragments, scFvs, or a combination thereof, wherein each Fab fragment and scFv comprises three heavy chain CDRs and three light chain CDRs that specifically bind a first antigen;
(b) obtaining a plurality of antibody Fab fragments, scFvs, or a combination thereof, wherein each Fab fragment and scFv comprises three heavy chain CDRs and three light chain CDRs that specifically bind a second antigen;
(c) cloning into a vector:
(i) the CDRs of the three heavy chain CDRs that specifically bind the first antigen,
(ii) the CDRs of the three heavy chain CDRs that specifically bind the second antigen, and
(iii) three light chain CDRs selected from the group consisting of
(a) the CDRs of the three light chain CDRs that specifically bind the first antigen,
(b) the CDRs of the three light chain CDRs that specifically bind the second antigen, and
(c) CDRs of three light chain CDRs that do not specifically bind either the first antigen or the second antigen;
wherein the vector(s) encode(s) for one type of multispecific antibody construct module and a plurality of vectors are generated that encode for a plurality of multispecific antibody constructs comprising the heavy chain CDRs that bind to each antigen and the light chain CDRs of (c)(iii);
(d) expressing each multispecific antibody construct in a mammalian host cell;
(e) purifying each multispecific antibody construct;
(f)(i) measuring the expression levels of each multispecific antibody construct and (ii) measuring the binding affinity of each multispecific antibody construct to the first antigen and the second antigen,
wherein steps (f)(i) and (f)(ii) can be performed simultaneously or in any order; and
(g) comparing the expression levels of (f)(i) and the binding affinities of (f)(ii) for each multispecific antibody construct in order to identify the optimal pairing of the three heavy chain CDRs that specifically bind a first antigen and the light chain CDRs of (c)(iii) with the optimal pairing of the three heavy chain CDRs that specifically bind a second antigen and the same light chain CDRs of (c)(iii).

In one embodiment, the plurality of antibody Fab fragments, scFvs, or a combination thereof, wherein each Fab fragment and scFv comprise three heavy chain CDRs and three light chain CDRs that specifically bind a first antigen is selected from the group consisting of at least two antibody Fab fragments, scFvs, or a combination thereof; at least three antibody Fab fragments, scFvs, or a combination thereof; at least four antibody Fab fragments, scFvs, or a combination thereof; at least five antibody Fab fragments, scFvs, or a combination thereof; at least six antibody Fab fragments, scFvs, or a combination thereof; at least seven antibody Fab fragments, scFvs, or a combination thereof; at least eight antibody Fab fragments, scFvs, or a combination thereof; at least nine antibody Fab fragments, scFvs, or a combination thereof; and at least ten antibody Fab fragments, scFvs, or a combination thereof.

In one embodiment, the plurality of antibody Fab fragments, scFvs, or a combination thereof, wherein each Fab fragment and scFv comprise three heavy chain CDRs and three light chain CDRs that specifically bind a second antigen is selected from the group consisting of at least two antibody Fab fragments, scFvs, or a combination thereof; at least three antibody Fab fragments, scFvs, or a combination thereof; at least four antibody Fab fragments, scFvs, or a combination thereof; at least five antibody Fab fragments, scFvs, or a combination thereof; at least six antibody Fab fragments, scFvs, or a combination thereof; at least seven antibody Fab fragments, scFvs, or a combination thereof; at least eight antibody Fab fragments, scFvs, or a combination thereof; at least nine antibody Fab fragments, scFvs, or a combination thereof; and at least ten antibody Fab fragments, scFvs, or a combination thereof.

In one embodiment, the multispecific antibody construct modules include at least two of the modules selected from the group consisting of Fab/Fab hetero Fc, scFab/scFab hetero Fc, Fab/scFv hetero Fc, Fab/Fab-scFv hetero Fc, Fab/scFv-Fab hetero Fc, Fab/Fab hetero Fc-scFv, IgG-Fab, scFab-Fc-Fab, IgG-scFv, scFv-IgG, and Fab-scFv-Fc.

In one embodiment, the mammalian host cell is selected from the group consisting of Chinese hamster ovary (“CHO”) cells, monkey kidney CV1 line transformed by SV40 (“COS-7”); human embryonic kidney line 293 (“HEK293”); baby hamster kidney cells (“BHK”); mouse sertoli cells (“TM4”); monkey kidney cells (“CV1”); African green monkey kidney cells (“VERO-76”); human cervical carcinoma cells (“HELA”); canine kidney cells (“MDCK”); buffalo rat liver cells (“BRL”); human lung cells (“W138”); human hepatoma cells (“Hep G2”); mouse mammary tumor (“MMT”); TRI cells; MRC 5 cells: and FS4 cells.

In one embodiment, the expression levels are determined by a method selected from the group consisting of A280 measurement SDS-PAGE, microchip capillary electrophoresis (MCE), Bradford assay, and bicinchoninic acid (BCA) assay.

In one embodiment, the binding affinity of each multispecific antibody construct to the first antigen and the second antigen is measured using Octet, Forte Bio, Carterra LSA, SPR and Flow cytometry.

In one embodiment, each multispecific antibody construct is purified by Protein A, Lambda and Kappa resins, and affinity tag purification.

In one aspect the present invention is directed to a method for selecting a multispecific antibody construct, the method comprising:
(a) obtaining a plurality of at least two antibody Fab fragments, scFvs, or a combination thereof, wherein each Fab fragment and scFv comprises three heavy chain CDRs and three light chain CDRs that specifically bind a first antigen;
(b) obtaining a plurality of at least two antibody Fab fragments, scFvs, or a combination thereof, wherein each Fab fragment and scFv comprises three heavy chain CDRs and three light chain CDRs that specifically bind a second antigen;
(c) cloning into a vector:
(i) the CDRs of the three heavy chain CDRs that specifically bind the first antigen,
(ii) the CDRs of the three heavy chain CDRs that specifically bind the second antigen, and
(iii) three light chain CDRs selected from the group consisting of
(a) the CDRs of the three light chain CDRs that specifically bind the first antigen,
(b) the CDRs of the three light chain CDRs that specifically bind the second antigen, and
(c) CDRs of three light chain CDRs that do not specifically either the first antigen or the second antigen;
wherein the vector(s) encode(s) for a multispecific antibody construct module and a plurality of vectors are generated that encode for a plurality of multispecific antibody constructs comprising the heavy chain CDRs that bind to each antigen and the light chain CDRs of (c)(iii);
(d) expressing each multispecific antibody construct in a mammalian host cell, wherein the mammalian host cell is selected from the group consisting of HEK293 cells and CHO cells;
(e) purifying each multispecific antibody construct using Protein A chromatography;
(f)(i) measuring the expression levels of each multispecific antibody construct using A280 measurement and (ii) measuring the binding affinity of each multispecific antibody construct to the first antigen and the second antigen using Octet,
wherein steps (f)(i) and (f)(ii) can be performed simultaneously or in any order; and
(g) comparing the expression levels of (f)(i) and the binding affinities of (f)(ii) for each multispecific antibody construct in order to identify the optimal pairing of the three heavy chain CDRs that specifically bind a first antigen and the light chain CDRs of (c)(iii) with the optimal pairing of the three heavy chain CDRs that specifically bind a second antigen and the same light chain CDRs of (c)(iii).

In one embodiment, the multispecific antibody construct modules include at least two of the modules selected from the group consisting of Fab/Fab hetero Fc, scFab/scFab hetero Fc, Fab/scFv hetero Fc, Fab/Fab-scFv hetero Fc, Fab/scFv-Fab hetero Fc, Fab/Fab hetero Fc-scFv, IgG-Fab, scFab-Fc-Fab, IgG-scFv, scFv-IgG, and Fab-scFv-Fc.

In one aspect the present invention is directed to a method for selecting a multispecific antibody construct, the method comprising:
(a) obtaining a plurality of antibody Fab fragments, scFvs, or a combination thereof, wherein each Fab fragment and scFv comprises three heavy chain CDRs and three light chain CDRs that specifically bind a first antigen;
(b) obtaining a plurality of antibody Fab fragments, scFvs, or a combination thereof, wherein each Fab fragment and scFv comprises three heavy chain CDRs and three light chain CDRs that specifically bind a second antigen;
(c) cloning the CDRs of the two pluralities into a vector(s) that encode(s) for a multispecific antibody construct module and a plurality of vectors are generated that encode a plurality of multispecific antibody constructs comprising the CDRs that bind to each antigen;
(d) expressing each multispecific antibody construct in a mammalian host cell;
(e) purifying each multispecific antibody construct;
(f)(i) measuring the expression levels of each multispecific antibody construct and (ii) calculating the percent of correct and incorrect multispecific antibody construct module species produced,
wherein steps (f)(i) and (f)(ii) can be performed simultaneously or in any order; and
(g) comparing the expression levels of (f)(i) and the percentage of correct and incorrect multispecific antibody construct module species of (f)(ii) for each multispecific antibody construct in order to identify the optimal pairing of three heavy chain CDRs and three light chain CDRs that specifically bind a first antigen with three heavy chain CDRs and three light chain CDRs that specifically bind a second antigen.

In one embodiment, the plurality of antibody Fab fragments, scFvs, or a combination thereof, wherein each Fab fragment and scFv comprise three heavy chain CDRs and three light chain CDRs that specifically bind a first antigen is selected from the group consisting of at least two antibody Fab fragments, scFvs, or a combination thereof; at least three antibody Fab fragments, scFvs, or a combination thereof; at least four antibody Fab fragments, scFvs, or a combination thereof; at least five antibody Fab fragments, scFvs, or a combination thereof; at least six antibody Fab fragments, scFvs, or a combination thereof; at least seven antibody Fab fragments, scFvs, or a combination thereof; at least eight antibody Fab fragments, scFvs, or a combination thereof; at least nine antibody Fab fragments, scFvs, or a combination thereof; and at least ten antibody Fab fragments, scFvs, or a combination thereof.

In one embodiment, the plurality of antibody Fab fragments, scFvs, or a combination thereof, wherein each Fab fragment and scFv comprise three heavy chain CDRs and three light chain CDRs that specifically bind a second antigen is selected from the group consisting of at least two antibody Fab fragments, scFvs, or a combination thereof; at least three antibody Fab fragments, scFvs, or a combination thereof; at least four antibody Fab fragments, scFvs, or a combination thereof; at least five antibody Fab fragments, scFvs, or a combination thereof; at least six antibody Fab fragments, scFvs, or a combination thereof; at least seven antibody Fab fragments, scFvs, or a combination thereof; at least eight antibody Fab fragments, scFvs, or a combination thereof; at least nine antibody Fab fragments, scFvs, or a combination thereof; and at least ten antibody Fab fragments, scFvs, or a combination thereof.

In one embodiment, the multispecific antibody construct module is selected from the group consisting of Fab/Fab hetero Fc, scFab/scFab hetero Fc, Fab/scFv hetero Fc, Fab/Fab-scFv hetero Fc, Fab/scFv-Fab hetero Fc, Fab/Fab hetero Fc-scFv, IgG-Fab, scFab-Fc-Fab, IgG-scFv, scFv-IgG, and Fab-scFv-Fc.

In one embodiment, the mammalian host cell is selected from the group consisting of Chinese hamster ovary (“CHO”) cells, monkey kidney CV1 line transformed by SV40 (“COS-7”); human embryonic kidney line 293 (“HEK293”); baby hamster kidney cells (“BHK”); mouse sertoli cells (“TM4”); monkey kidney cells (“CV1”); African green monkey kidney cells (“VERO-76”); human cervical carcinoma cells (“HELA”); canine kidney cells (“MDCK”); buffalo rat liver cells (“BRL”); human lung cells (“W138”); human hepatoma cells (“Hep G2”); mouse mammary tumor (“MMT”); TRI cells; MRC 5 cells: and FS4 cells.

In one embodiment, the expression levels are determined by a method selected from the group consisting of A280 measurement SDS-PAGE, microchip capillary electrophoresis (MCE), Bradford assay, and bicinchoninic acid (BCA) assay.

In one embodiment, the percentage of correct and incorrect multispecific antibody construct modality species is determined by a method selected from the group consisting of liquid chromatography-mass spectrometry (“LC-MS”), Caliper, HPLC SEC, SDS-PAGE, and microchip capillary electrophoresis (“MCE”).

In one embodiment, each multispecific antibody construct is purified by Protein A, Lambda and Kappa resins, and affinity tag purification.

In one aspect the present invention is directed to a method for selecting a multispecific antibody construct, the method comprising:
(a) obtaining a plurality of at least two antibody Fab fragments, scFvs, or a combination thereof, wherein each Fab fragment and scFv comprises three heavy chain CDRs and three light chain CDRs that specifically bind a first antigen;
(b) obtaining a plurality of at least two antibody Fab fragments, scFvs, or a combination thereof, wherein each Fab fragment and scFv comprises three heavy chain CDRs and three light chain CDRs that specifically bind a second antigen;
(c) cloning the CDRs of the two pluralities into a vector(s) that encode(s) for a multispecific antibody construct module and a plurality of vectors are generated that encode a plurality of multispecific antibody constructs that bind to each antigen;
(d) expressing each multispecific antibody construct in a mammalian host cell, wherein the mammalian host cell is selected from the group consisting of HEK293 cells and CHO cells;
(e) purifying each multispecific antibody construct using Protein A chromatography;
(f)(i) measuring the expression levels of each multispecific antibody construct using A280 measurement and (ii) calculating the percent of correct and incorrect multispecific antibody construct module species produced using liquid chromatography-mass spectrometry (“LC-MS”),
wherein steps (f)(i) and (f)(ii) can be performed simultaneously or in any order; and
(g) comparing the expression levels of (f)(i) and the percentage of correct and incorrect multispecific antibody construct module species of (f)(ii) for each multispecific antibody construct in order to identify the optimal pairing of three heavy chain CDRs and three light chain CDRs that specifically bind a first antigen with three heavy chain CDRs and three light chain CDRs that specifically bind a second antigen.

In one embodiment, the multispecific antibody construct module is selected from the group consisting of Fab/Fab hetero Fc, scFab/scFab hetero Fc, Fab/scFv hetero Fc, Fab/Fab-scFv hetero Fc, Fab/scFv-Fab hetero Fc, Fab/Fab hetero Fc-scFv, IgG-Fab, scFab-Fc-Fab, IgG-scFv, scFv-IgG, and Fab-scFv-Fc.

In one aspect the present invention is directe to a method for selecting a multispecific antibody construct, the method comprising:
(a) obtaining a first antibody Fab fragment or scFv, wherein each Fab fragment or scFv comprises three heavy chain CDRs and three light chain CDRs that specifically bind a first antigen;
(b) obtaining a second antibody Fab fragment or scFv, wherein each Fab fragment or scFv comprises three heavy chain CDRs and three light chain CDRs that specifically bind a second antigen;
(c) cloning into a vector:
(i) the CDRs of the three heavy chain CDRs that specifically bind the first antigen,
(ii) the CDRs of the three heavy chain CDRs that specifically bind the second antigen, and
(iii) three light chain CDRs selected from the group consisting of
(a) the CDRs of the three light chain CDRs that specifically bind the first antigen,
(b) the CDRs of the three light chain CDRs that specifically bind the second antigen, and
(c) CDRs of three light chain CDRs that do not specifically either the first antigen or the second antigen;
wherein the vector(s) encode(s) for more than one type of multispecific antibody construct module and a plurality of vectors are generated that encode for a plurality of multispecific antibody constructs of different modules comprising the heavy chain CDRs that bind to each antigen and the light chain CDRs of (c)(iii);
(d) expressing each multispecific antibody construct of the different modules in a mammalian host cell;
(e) purifying each multispecific antibody construct;
(f)(i) measuring the expression levels of each multispecific antibody construct and (ii) measuring the binding affinity of each multispecific antibody construct to the first antigen and the second antigen,
wherein steps (f)(i) and (f)(ii) can be performed simultaneously or in any order; and
(g) comparing the expression levels of (f)(i) and the binding affinities of (f)(ii) for each multispecific antibody construct in order to identify the optimal module for pairing of the three heavy chain CDRs that specifically bind a first antigen and the light chain CDRs of (c)(iii) with the three heavy chain CDRs that specifically bind a second antigen and the same light chain CDRs of (c)(iii).

In one embodiment, the multispecific antibody construct modules include at least two of the modules selected from the group consisting of Fab/Fab hetero Fc, scFab/scFab hetero Fc, Fab/scFv hetero Fc, Fab/Fab-scFv hetero Fc, Fab/scFv-Fab hetero Fc, Fab/Fab hetero Fc-scFv, IgG-Fab, scFab-Fc-Fab, IgG-scFv, scFv-IgG, and Fab-scFv-Fc.

In one embodiment, the mammalian host cell is selected from the group consisting of Chinese hamster ovary (“CHO”) cells, monkey kidney CV1 line transformed by SV40 (“COS-7”); human embryonic kidney line 293 (“HEK293”); baby hamster kidney cells (“BHK”); mouse sertoli cells (“TM4”); monkey kidney cells (“CV1”); African green monkey kidney cells (“VERO-76”); human cervical carcinoma cells (“HELA”); canine kidney cells (“MDCK”); buffalo rat liver cells (“BRL”); human lung cells (“W138”); human hepatoma cells (“Hep G2”); mouse mammary tumor (“MMT”); TRI cells; MRC 5 cells: and FS4 cells.

In one embodiment, the expression levels are determined by a method selected from the group consisting of A280 measurement SDS-PAGE, microchip capillary electrophoresis (MCE), Bradford assay, and bicinchoninic acid (BCA) assay.

In one embodiment, binding affinity of each multispecific antibody construct to the first antigen and the second antigen is measured using Octet, Forte Bio, Carterra LSA, SPR and Flow cytometry.

In one embodiment, each multispecific antibody construct is purified by Protein A, Lambda and Kappa resins, and affinity tag purification.

In one aspect the present invention is directed to a method for selecting a multispecific antibody construct, the method comprising:
(a) obtaining a first antibody Fab fragment or scFv, wherein each Fab fragment or scFv comprises three heavy chain CDRs and three light chain CDRs that specifically bind a first antigen;
(b) obtaining a second antibody Fab fragment or scFv, wherein each Fab fragment or scFv comprises three heavy chain CDRs and three light chain CDRs that specifically bind a second antigen;
(c) cloning into a vector:
(i) the CDRs of the three heavy chain CDRs that specifically bind the first antigen,
(ii) the CDRs of the three heavy chain CDRs that specifically bind the second antigen, and
(iii) three light chain CDRs selected from the group consisting of
(a) the CDRs of the three light chain CDRs that specifically bind the first antigen,
(b) the CDRs of the three light chain CDRs that specifically bind the second antigen, and
(c) CDRs of three light chain CDRs that do not specifically either the first antigen or the second antigen;
wherein the vector(s) encode(s) for more than one type of multispecific antibody construct module and a plurality of vectors are generated that encode for a plurality of multispecific antibody constructs of different modules comprising the heavy chain CDRs that bind to each antigen and the light chain CDRs of (c)(iii);
(d) expressing each multispecific antibody construct in a mammalian host cell, wherein the mammalian host cell is selected from the group consisting of HEK293 cells and CHO cells;
(e) purifying each multispecific antibody construct using Protein A chromatography;
(f)(i) measuring the expression levels of each multispecific antibody construct using A280 measurement and (ii) measuring the binding affinity of each multispecific antibody construct to the first antigen and the second antigen using Octet,
(g) comparing the expression levels of (f)(i) and the binding affinities of (f)(ii) for each multispecific antibody construct in order to identify the optimal module for pairing of the three heavy chain CDRs that specifically bind a first antigen and the light chain CDRs of (c)(iii) with the three heavy chain CDRs that specifically bind a second antigen and the same light chain CDRs of (c)(iii).

In one embodiment, the multispecific antibody construct modules include at least two of the modules selected from the group consisting of Fab/Fab hetero Fc, scFab/scFab hetero Fc, Fab/scFv hetero Fc, Fab/Fab-scFv hetero Fc, Fab/scFv-Fab hetero Fc, Fab/Fab hetero Fc-scFv, IgG-Fab, scFab-Fc-Fab, IgG-scFv, scFv-IgG, and Fab-scFv-Fc.

In one aspect the present invention is directed to a method for selecting a multispecific antibody construct, the method comprising:
(a) obtaining a first antibody Fab fragment or scFv, wherein each Fab fragment or scFv comprises three heavy chain CDRs and three light chain CDRs that specifically bind a first antigen;
(b) obtaining a second antibody Fab fragment or scFv, wherein each Fab fragment or scFv comprises three heavy chain CDRs and three light chain CDRs that specifically bind a second antigen;
(c) cloning the CDRs of the first antibody Fab fragment or scFv and the second antibody Fab fragment or scFv into a vector(s),
wherein the vector(s) encode(s) for more than one type of multispecific antibody construct module and a plurality of vectors are generated that encode for a plurality of multispecific antibody constructs of different modules comprising the CDRs that bind to each antigen;
(d) expressing each multispecific antibody construct in a mammalian host cell;
(e) purifying each multispecific antibody construct;
(f)(i) measuring the expression levels of each multispecific antibody construct and (ii) calculating the percent of correct and incorrect multispecific antibody construct module species produced,
wherein steps (f)(i) and (f)(ii) can be performed simultaneously or in any order; and
(g) comparing the expression levels of (f)(i) and the percentage of correct and incorrect multispecific antibody construct module species of (f)(ii) for each multispecific antibody construct in order to identify the optimal multispecific antibody construct module for pairing of the three heavy chain CDRs and three light chain CDRs that specifically bind a first antigen with the three heavy chain CDRs and three light chain CDRs that specifically bind a second antigen.

In one embodiment, the multispecific antibody construct modules include at least two of the modules selected from the group consisting of Fab/Fab hetero Fc, scFab/scFab hetero Fc, Fab/scFv hetero Fc, Fab/Fab-scFv hetero Fc, Fab/scFv-Fab hetero Fc, Fab/Fab hetero Fc-scFv, IgG-Fab, scFab-Fc-Fab, IgG-scFv, scFv-IgG, and Fab-scFv-Fc.

In one embodiment, the mammalian host cell is selected from the group consisting of Chinese hamster ovary (“CHO”) cells, monkey kidney CV1 line transformed by SV40 (“COS-7”); human embryonic kidney line 293 (“HEK293”); baby hamster kidney cells (“BHK”); mouse sertoli cells (“TM4”); monkey kidney cells (“CV1”); African green monkey kidney cells (“VERO-76”); human cervical carcinoma cells (“HELA”); canine kidney cells (“MDCK”); buffalo rat liver cells (“BRL”); human lung cells (“W138”); human hepatoma cells (“Hep G2”); mouse mammary tumor (“MMT”); TRI cells; MRC 5 cells: and FS4 cells.

In one embodiment, the expression levels are determined by a method selected from the group consisting of A280 measurement SDS-PAGE, microchip capillary electrophoresis (MCE), Bradford assay, and bicinchoninic acid (BCA) assay.

In one embodiment, the percentage of correct and incorrect multispecific antibody construct modality species is determined by a method selected from the group consisting of liquid chromatography-mass spectrometry (“LC-MS”), Caliper, HPLC SEC, SDS-PAGE, and microchip capillary electrophoresis (“MCE”).

In one embodiment, each multispecific antibody construct is purified by Protein A, Lambda and Kappa resins, and affinity tag purification.

In one aspect the present invention is directed to a method for selecting a multispecific antibody construct, the method comprising:
(a) obtaining a first antibody Fab fragment or scFv, wherein each Fab fragment or scFv comprises three heavy chain CDRs and three light chain CDRs that specifically bind a first antigen;
(b) obtaining a second antibody Fab fragment or scFv, wherein each Fab fragment or scFv comprises three heavy chain CDRs and three light chain CDRs that specifically bind a second antigen;
(c) cloning the CDRs of the first antibody Fab fragment or scFv and the second antibody Fab fragment or scFv into a vector(s),
wherein the vector(s) encode(s) for more than one type of multispecific antibody construct module and a plurality of vectors are generated that encode for a plurality of multispecific antibody constructs of different modules comprising the CDRs that bind to each antigen;
(d) expressing each multispecific antibody construct in a mammalian host cell, wherein the mammalian host cell is selected from the group consisting of HEK293 cells and CHO cells;
(e) purifying each multispecific antibody construct using Protein A chromatography;
(f)(i) measuring the expression levels of each multispecific antibody construct using A280 and (ii) calculating the percent of correct and incorrect multispecific antibody construct module species produced using liquid chromatography-mass spectrometry (“LC-MS”),
wherein steps (f)(i) and (f)(ii) can be performed simultaneously or in any order; and
(g) comparing the expression levels of (f)(i) and the percentage of correct and incorrect multispecific antibody construct module species of (f)(ii) for each multispecific antibody construct in order to identify the optimal multispecific antibody construct module for pairing of the three heavy chain CDRs and three light chain CDRs that specifically bind a first antigen with the three heavy chain CDRs and three light chain CDRs that specifically bind a second antigen.

In one embodiment, the multispecific antibody construct modules include at least two of the modules selected from the group consisting of Fab/Fab hetero Fc, scFab/scFab hetero Fc, Fab/scFv hetero Fc, Fab/Fab-scFv hetero Fc, Fab/scFv-Fab hetero Fc, Fab/Fab hetero Fc-scFv, IgG-Fab, scFab-Fc-Fab, IgG-scFv, scFv-IgG, and Fab-scFv-Fc.

### BRIEF DESCRIPTION OF THE DRAWINGS

FIG. 1 depicts a structural analysis of HC-LC interface. A) Schematic illustration of an IgG structural configuration and surface representation of the Fv region. In the Fab arm colored in blue, the Fv (VH/VL) interface, CH1/CL interface and CDR regions are each circled by dash lines. The surface of VH and VL domains of an IgG (PDB:1HZH) was created using PyMol with a color code for each CDR. The interface between VH and VL is highlighted with a dash line. B) Calculation of HC/LC interfacing residues using 6 structures performed by PDBePISA²⁹. The number of interfacing residues in the corresponding regions are listed. C) Sequence alignment of 508 human antibodies with structures deposited in Protein Data Bank (PDB) highlighting the CDRs diversity.

FIG. 2 depicts a schematic representation of the design of high-throughput Chain Selective Assessment methods (CSA). A) Schematic representation of high-throughput CSA. Two panels of parental mAbs against Target-A (in blue) and Target-B (in green) are subjected to high-throughput screening with 2 possible outcomes, 4-chain Hetero-Fcs and cLC Hetero-Fcs. B) Schematic and timeline of the 2 high-throughput CSA methods. In the competition CSA (cCSA) experiment, each anti-Target-A mAb is combined with every anti-Target-B mAb. To ensure HC heterodimerization CPMs represented in red and blue dots were engineered into the CH3 domains. Thus, 4 HC/LC pairing scenarios are left to the native properties within the chains interface. All combinations of 2 HCs and 2 LCs are co-transfected into HEK293-6E cells, followed by ProA purification and LC-MS quantitation of each IgG species. A sample with high percentage of correct MW IgG species will indicate that the corresponding antibody combination have high preference for cognate HC/LC pairing while minimizing cross-pairing. In the non-competition CSA (ncCSA) experiment, non-cognate HC/LC pairs are tested for expression in HEK293-6E cells, followed by ProA purification and determination of protein concentration (A280). High level of hybrid IgGs expression will suggest promiscuous LCs to be selected for cLC Hetero-Fcs assemble together with the cognate and non-cognate HCs. To identify positive binders, purified hybrid IgGs are further analyzed by ForteBio Octet HTX.

FIG. 3 depicts a high-throughput screening for low-crosspairing antibody combinations in cCSA experiment. A) Analysis of protein expression level. Twelve parental mAbs (8 anti-Target-A and 4 anti-Target-B) and the 32 resulting Hetero-Fc combinations were transiently expressed in HEK293-6E cells. The molecules in the conditioned media were purified with a high-throughput KingFisher Flex system, followed by measuring protein concentration (A280). Expression levels are represented by ProA yields calculated in milligrams (mg) per liter of conditioned medium. B) Representative high-resolution LC-MS analysis for 2 ProA purified samples (A3×B4 and A4×B2) highlighted with schematics showing correct and mispaired IgG species. C) The percentage of IgG species with Hetero-HCs was calculated from LC-MS data and plotted. Percentage of IgG species with correct MW (1×HC1+1×LC1+1×HC2+1×LC2) reflects the correct HC/LC pairing. *, small MW difference where LC-MS unable to distinguish IgG species.

FIG. 4 depicts predictability of HC/LC pairing in 4-chain Hetero-Fcs. A) Final yields observed after CIEX purification with a purity target of >90%. The yields of Hetero-Fcs and their corresponding parental mAbs were plotted side-by-side. B) Receiver operating characteristic curve (ROC) plot of the CIEX yields and % of correct IgG species. C) Correlational analysis of CIEX yields and HC/LC pairing. The dash line indicates 50% benchmark of correct IgG species determined by LC-MS. D) CIEX chromatographs of two representative molecules. E) Breakdown of number of molecules in each step of cCSA experiment.

FIG. 5 depicts a high-throughput screening for cLC Hetero-Fcs by ncCSA. A) Relative expression of 144 non-cognate HC/LC pairs in two bispecific panels (A×B and C×B). ProA yield of each non-cognate HC/LC pair was normalized to that of the corresponding cognate HC/LC pair (mAb) control. A non-cognate HC/LC pair with relative expression level >0.5 suggest that its LC is promiscuous to the paired non-cognate HC and it becomes selected to assemble cLC Hetero-Fc together with cognate HC. B) Selected ProA purified non-cognate HC/LC pairs and mAb controls were analyzed by non-reducing SDS-PAGE gel and measured by A280 to determine yields. C) Schematics of 2 possible cLC Hetero-Fcs. CPMs are shown as red and blue dots in the CH3 domains. D) and E) Expression levels of cLC Hetero-Fcs for the two bispecific panels, A×B and C×B, respectively, represented by ProA yields calculated in mg per liter of conditioned media. Dash lines highlight 60 mg/L. F) Heatmap plot of the relative binding affinity of cLC Hetero-Fcs to their cognate and non-cognate antigens. The binding affinity (K_{D}) of 106 cLC Hetero-Fcs and 22 corresponding mAbs to soluble antigen-A, -B or -C was measured by ForteBio Octet. Then, the relative binding affinity of cLC Hetero-Fcs compared to that of the corresponding cognate and non-cognate HC mAbs were calculated and plotted. G) Inverted pyramid diagram showing number of molecules in each step of ncCSA experiment.

FIG. 6 depicts expression, purification and binding properties of two selected cLC Hetero-Fc molecules. A) Final CIEX yields of two cLC Hetero-Fcs (A2×B4 and C4×B3) and their corresponding parental mAbs. B) CIEX chromatographs for A2×B4 and C4×B3. C-E) Binding kinetics of two cLC Hetero-Fcs (A2×B4 and C4×B3) and respective controls (two hybrid IgGs (HC-A2/LC-B4 and HC-C4/LC-B3) and two parental mAbs (B4 and B3)). All binding kinetics sensorgrams show processed data overlaid with the global fit to a 1:1 binding model. The weaker binding to antigen-C is rapid equilibrium with a lack of curvature leading to the larger variance in replicate measurements. The binding affinity (K_{D}) was calculated as mean±SD from three independent measurements.

FIG. 7 depicts multispecific antibody construct modules.

FIG. 8 depicts a summary of purification and analytics of 32 4-chain Hetero-Fcs and corresponding parental mAbs.

FIG. 9 depicts relative expression of 144 non-cognate HC/LC pairs in two bispecific programs (A×B and C×B). ProA yield of each non-cognate HC/LC pair was normalized to that of the corresponding cognate HC/LC pair (mAb) control and plotted.

FIG. 10 depicts expression of non-cognate HC/LC pairs and parental mAbs (highlighted in grey) in non-competition CSA experiment.

FIG. 11 depicts expression of anti-Target-C parental mAbs.

FIG. 12 depicts high-resolution LC-MS analysis of ProA purified cLC Hetero-IgGs, for the two bispecific programs, A×B (A) and C×B (B). The percentage of IgG species with correct MW was calculated from LC-MS data and plotted. The average values of cLC Hetero-IgGs and 4-chain Hetero-IgGs (FIG. 3C) are highlighted by dash lines.

FIG. 13 depicts binding kinetics of parental mAbs to their corresponding antigens.

FIG. 14 depicts a summary of the purification and analytics of two cLC Hetero-IgGs and their parental mAbs.

FIG. 15 depicts schematics of 1 correctly assembled IgG species and 9 mispaired IgG species after co-expression of 2 different HCs and 2 different LCs in a single cell.

FIG. 16 depicts expression of parental mAbs and 4-chain Hetero-IgGs in competition CSA experiment. A) Analysis of ProA purified proteins by non-reducing SDS-PAGE gels. B), Analysis of protein expression levels shown in milligrams per liter after ProA purification step.

FIG. 17 depicts relative binding affinity of 4-chain Hetero-IgGs compared to parental mAbs. The binding affinity (K_{D}) of 11 SP-purified Hetero-IgGs and their corresponding mAbs to soluble antigen-A and/or antigen-B were measured by Fortebio Octet. Then, the relative binding affinity of Hetero-IgGs compared to the corresponding parental mAbs were calculated and plotted.

### DETAILED DESCRIPTION OF THE INVENTION

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

The term “and/or” wherever used herein includes the meaning of “and”, “or” and “all or any other combination of the elements connected by said term”.

The term “about” or “approximately” as used herein means within ±20%, preferably within ±15%, more preferably within ±10%, and most preferably within ±5% of a given value or range.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim.

In each instance herein any of the terms “comprising”, “consisting essentially of” and “consisting of” may be replaced with either of the other two terms.

As used herein, the term “antigen binding protein” refers to a protein that specifically binds to one or more target antigens. An antigen binding protein can include an antibody and functional fragments thereof. A “functional antibody fragment” is a portion of an antibody that lacks at least some of the amino acids present in a full-length heavy chain and/or light chain, but which is still capable of specifically binding to an antigen. A functional antibody fragment includes, but is not limited to, a Fab fragment, a Fab′ fragment, a F(ab′)₂ fragment, a Fv fragment, a Fd fragment, and a complementarity determining region (CDR) fragment, and can be derived from any mammalian source, such as human, mouse, rat, rabbit, or camelid. Functional antibody fragments may compete for binding of a target antigen with an intact antibody and the fragments may be produced by the modification of intact antibodies (e.g. enzymatic or chemical cleavage) or synthesized de novo using recombinant DNA technologies or peptide synthesis.

An antigen binding protein can also include a protein comprising one or more functional antibody fragments incorporated into a single polypeptide chain or into multiple polypeptide chains. For instance, antigen binding proteins can include, but are not limited to, a single chain Fv (scFv), a diabody (see, e.g., EP 404,097; WO 93/11161; and Hollinger et al., Proc. Natl. Acad. Sci. USA, Vol. 90:6444-6448, 1993); an intrabody; a domain antibody (single VL or VH domain or two or more VH domains joined by a peptide linker; see Ward et al., Nature, Vol. 341:544-546, 1989); a maxibody (2 scFvs fused to Fc region, see Fredericks et al., Protein Engineering, Design & Selection, Vol. 17:95-106, 2004 and Powers et al., Journal of Immunological Methods, Vol. 251:123-135, 2001); a triabody; a tetrabody; a minibody (scFv fused to CH3 domain; see Olafsen et al., Protein Eng Des Sel., Vol. 17:315-23, 2004); a peptibody (one or more peptides attached to an Fc region, see WO 00/24782); a linear antibody (a pair of tandem Fd segments (VH-CH1-VH-CH1) which, together with complementary light chain polypeptides, form a pair of antigen binding regions, see Zapata et al., Protein Eng., Vol. 8:1057-1062, 1995); a small modular immunopharmaceutical (see U.S. Patent Publication No. 20030133939); and immunoglobulin fusion proteins (e.g. IgG-scFv, IgG-Fab, 2scFv-IgG, 4scFv-IgG, VH-IgG, IgG-VH, and Fab-scFv-Fc).

“Multispecific” means that an antigen binding protein is capable of specifically binding to two or more different antigens. “Bispecific” means that an antigen binding protein is capable of specifically binding to two different antigens. As used herein, an antigen binding protein “specifically binds” to a target antigen when it has a significantly higher binding affinity for, and consequently is capable of distinguishing, that antigen, compared to its affinity for other unrelated proteins, under similar binding assay conditions. Antigen binding proteins that specifically bind an antigen may have an equilibrium dissociation constant (K_{D})≤1×10⁻⁶ M. The antigen binding protein specifically binds antigen with “high affinity” when the K_{D} is ≤1×10⁻⁸ M.

Affinity is determined using a variety of techniques, an example of which is an affinity ELISA assay. In various embodiments, affinity is determined by a surface plasmon resonance assay (e.g., BIAcore®-based assay). Using this methodology, the association rate constant (kₐ in M⁻¹s⁻¹) and the dissociation rate constant (k_{d} in s⁻¹) can be measured. The equilibrium dissociation constant (K_{D} in M) can then be calculated from the ratio of the kinetic rate constants (k_{d}/kₐ). In some embodiments, affinity is determined by a kinetic method, such as a Kinetic Exclusion Assay (KinExA) as described in Rathanaswami et al. Analytical Biochemistry, Vol. 373:52-60, 2008. Using a KinExA assay, the equilibrium dissociation constant (K_{D} in M) and the association rate constant (kₐ in M⁻¹s⁻¹) can be measured. The dissociation rate constant (k_{d} in s⁻¹) can be calculated from these values (K_{D}×kₐ). In other embodiments, affinity is determined by an equilibrium/solution method. In certain embodiments, affinity is determined by a FACS binding assay.

In some embodiments, the multispecific antigen binding proteins described herein exhibit desirable characteristics such as binding avidity as measured by k_{d} (dissociation rate constant) of about 10⁻², 10⁻¹, 10⁻⁴, 10⁻¹, 10⁻⁶, 10⁻⁷, 10⁻⁸, 10⁻⁹, 10⁻¹⁰ s⁻¹ or lower (lower values indicating higher binding avidity), and/or binding affinity as measured by K_{D} (equilibrium dissociation constant) of about 10⁻⁹, 10⁻¹⁰, 10⁻¹¹, 10⁻¹², 10⁻¹³, 10⁻¹⁴, 10⁻¹, 10⁻¹⁶ M or lower (lower values indicating higher binding affinity).

As used herein, the term “antigen binding domain,” which is used interchangeably with “binding domain,” refers to the region of the antigen binding protein that contains the amino acid residues that interact with the antigen and confer on the antigen binding protein its specificity and affinity for the antigen.

As used herein, the term “CDR” refers to the complementarity determining region (also termed “minimal recognition units” or “hypervariable region”) within antibody variable sequences. There are three heavy chain variable region CDRs (CDRH1, CDRH2 and CDRH3) and three light chain variable region CDRs (CDRL1, CDRL2 and CDRL3). The term “CDR region” as used herein refers to a group of three CDRs that occur in a single variable region (i.e. the three-light chain CDRs or the three-heavy chain CDRs). The CDRs in each of the two chains typically are aligned by the framework regions to form a structure that binds specifically with a specific epitope or domain on the target protein. From N-terminus to C-terminus, naturally-occurring light and heavy chain variable regions both typically conform with the following order of these elements: FR1, CDR1, FR2, CDR2, FR3, CDR3 and FR4. A numbering system has been devised for assigning numbers to amino acids that occupy positions in each of these domains. This numbering system is defined in Kabat Sequences of Proteins of Immunological Interest (1987 and 1991, NIH, Bethesda, MD), or Chothia & Lesk, 1987, J. Mol. Biol. 196:901-917; Chothia et al., 1989, Nature 342:878-883. Complementarity determining regions (CDRs) and framework regions (FR) of a given antibody may be identified using this system.

In some embodiments of the multispecific antigen binding proteins of the invention, the binding domains comprise a Fab, a Fab′, a F(ab′)₂, a Fv, a single-chain variable fragment (scFv), or a nanobody. In one embodiment, both binding domains are Fab fragments. In another embodiment, one binding domain is a Fab fragment and the other binding domain is a scFv.

Papain digestion of antibodies produces two identical antigen-binding fragments, called “Fab” fragments, each with a single antigen-binding site, and a residual “Fc” fragment which contains the immunoglobulin constant region. The Fab fragment contains all of the variable domain, as well as the constant domain of the light chain and the first constant domain (CH1) of the heavy chain. Thus, a “Fab fragment” is comprised of one immunoglobulin light chain (light chain variable region (VL) and constant region (CL)) and the CH1 region and variable region (VH) of one immunoglobulin heavy chain. The heavy chain of a Fab molecule cannot form a disulfide bond with another heavy chain molecule. The Fc fragment displays carbohydrates and is responsible for many antibody effector functions (such as binding complement and cell receptors), that distinguish one class of antibody from another. The “Fd fragment” comprises the VH and CH1 domains from an immunoglobulin heavy chain. The Fd fragment represents the heavy chain component of the Fab fragment.

A “Fab′ fragment” is a Fab fragment having at the C-terminus of the CH1 domain one or more cysteine residues from the antibody hinge region.

A “F(ab′)₂ fragment” is a bivalent fragment including two Fab′ fragments linked by a disulfide bridge between the heavy chains at the hinge region.

The “Fv” fragment is the minimum fragment that contains a complete antigen recognition and binding site from an antibody. This fragment consists of a dimer of one immunoglobulin heavy chain variable region (VH) and one immunoglobulin light chain variable region (VL) in tight, non-covalent association. It is in this configuration that the three CDRs of each variable region interact to define an antigen binding site on the surface of the VH-VL dimer. A single light chain or heavy chain variable region (or half of an Fv fragment comprising only three CDRs specific for an antigen) has the ability to recognize and bind antigen, although at a lower affinity than the entire binding site comprising both VH and VL.

A “single-chain variable antibody fragment” or “scFv fragment” comprises the VH and VL regions of an antibody, wherein these regions are present in a single polypeptide chain, and optionally comprising a peptide linker between the VH and VL regions that enables the Fv to form the desired structure for antigen binding (see e.g., Bird et al., Science, Vol. 242:423-426, 1988; and Huston et al., Proc. Natl. Acad. Sci. USA, Vol. 85:5879-5883, 1988).

In particular, embodiments of the multispecific antigen binding proteins of the invention, the binding domains comprise an immunoglobulin heavy chain variable region (VH) and an immunoglobulin light chain variable region (VL) of an antibody or antibody fragment which specifically binds to the desired antigen.

The “variable region,” used interchangeably herein with “variable domain” (variable region of a light chain (VL), variable region of a heavy chain (VH)) refers to the region in each of the light and heavy immunoglobulin chains which is involved directly in binding the antibody to the antigen. As discussed above, the regions of variable light and heavy chains have the same general structure and each region comprises four framework (FR) regions whose sequences are widely conserved, connected by three CDRs. The framework regions adopt a beta-sheet conformation and the CDRs may form loops connecting the beta-sheet structure. The CDRs in each chain are held in their three-dimensional structure by the framework regions and form, together with the CDRs from the other chain, the antigen binding site.

The binding domains that specifically bind to target antigens can be derived a) from known antibodies to these antigens or b) from new antibodies or antibody fragments obtained by de novo immunization methods using the antigen proteins or fragments thereof, by phage display, or other routine methods. The antibodies from which the binding domains for the multispecific antigen binding proteins are derived can be monoclonal antibodies, polyclonal antibodies, recombinant antibodies, human antibodies, or humanized antibodies. In certain embodiments, the antibodies from which the binding domains are derived are monoclonal antibodies. In these and other embodiments, the antibodies are human antibodies or humanized antibodies and can be of the IgG1-, IgG2-, IgG3-, or IgG4-type.

The term “monoclonal antibody” (or “mAb”) as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against an individual antigenic site or epitope, in contrast to polyclonal antibody preparations that typically include different antibodies directed against different epitopes. Monoclonal antibodies may be produced using any technique known in the art, e.g., by immortalizing spleen cells harvested from the transgenic animal after completion of the immunization schedule. The spleen cells can be immortalized using any technique known in the art, e.g., by fusing them with myeloma cells to produce hybridomas. Myeloma cells for use in hybridoma-producing fusion procedures preferably are non-antibody-producing, have high fusion efficiency, and enzyme deficiencies that render them incapable of growing in certain selective media which support the growth of only the desired fused cells (hybridomas). Examples of suitable cell lines for use in mouse fusions include Sp-20, P3-X63/Ag8, P3-X63-Ag8.653, NS1/1.Ag 4 1, Sp210-Ag14, FO, NSO/U, MPC-11, MPC11-X45-GTG 1.7 and S194/5XXO Bul; examples of cell lines used in rat fusions include R210.RCY3, Y3-Ag 1.2.3, IR983F and 4B210. Other cell lines useful for cell fusions are U-266, GM1500-GRG2, LICR-LON-HMy2 and UC729-6.

In some instances, a hybridoma cell line is produced by immunizing an animal (e.g., a transgenic animal having human immunoglobulin sequences) with target antigen; harvesting spleen cells from the immunized animal; fusing the harvested spleen cells to a myeloma cell line, thereby generating hybridoma cells; establishing hybridoma cell lines from the hybridoma cells, and identifying a hybridoma cell line that produces an antibody that binds target antigen.

Monoclonal antibodies secreted by a hybridoma cell line can be purified using any technique known in the art, such as protein A-Sepharose, hydroxylapatite chromatography, gel electrophoresis, dialysis, or affinity chromatography. Hybridomas or mAbs may be further screened to identify mAbs with particular properties, such as the ability to bind cells expressing target antigen, ability to block or interfere with the binding of the target antigen ligand to their respective receptors, or the ability to functionally block either of the receptors, e.g., a cAMP assay.

In some embodiments, the binding domains of the multispecific antigen binding proteins of the invention may be derived from humanized antibodies. A “humanized antibody” refers to an antibody in which regions (e.g. framework regions) have been modified to comprise corresponding regions from a human immunoglobulin. Generally, a humanized antibody can be produced from a monoclonal antibody raised initially in a non-human animal. Certain amino acid residues in this monoclonal antibody, typically from non-antigen recognizing portions of the antibody, are modified to be homologous to corresponding residues in a human antibody of corresponding isotype. Humanization can be performed, for example, using various methods by substituting at least a portion of a rodent variable region for the corresponding regions of a human antibody (see, e.g., U.S. Pat. Nos. 5,585,089 and 5,693,762; Jones et al., Nature, Vol. 321:522-525, 1986; Riechmann et al., Nature, Vol. 332:323-27, 1988; Verhoeyen et al., Science, Vol. 239:1534-1536, 1988). The CDRs of light and heavy chain variable regions of antibodies generated in another species can be grafted to consensus human FRs. To create consensus human FRs, FRs from several human heavy chain or light chain amino acid sequences may be aligned to identify a consensus amino acid sequence.

New antibodies generated against the target antigen from which binding domains for the multispecific antigen binding proteins of the invention can be derived can be fully human antibodies. A “fully human antibody” is an antibody that comprises variable and constant regions derived from or indicative of human germ line immunoglobulin sequences. One specific means provided for implementing the production of fully human antibodies is the “humanization” of the mouse humoral immune system. Introduction of human immunoglobulin (Ig) loci into mice in which the endogenous Ig genes have been inactivated is one means of producing fully human monoclonal antibodies (mAbs) in mouse, an animal that can be immunized with any desirable antigen. Using fully human antibodies can minimize the immunogenic and allergic responses that can sometimes be caused by administering mouse or mouse-derived mAbs to humans as therapeutic agents.

Fully human antibodies can be produced by immunizing transgenic animals (usually mice) that are capable of producing a repertoire of human antibodies in the absence of endogenous immunoglobulin production. Antigens for this purpose typically have six or more contiguous amino acids, and optionally are conjugated to a carrier, such as a hapten. See, e.g., Jakobovits et al., 1993, Proc. Natl. Acad. Sci. USA 90:2551-2555; Jakobovits et al., 1993, Nature 362:255-258; and Bruggermann et al., 1993, Year in Immunol. 7:33. In one example of such a method, transgenic animals are produced by incapacitating the endogenous mouse immunoglobulin loci encoding the mouse heavy and light immunoglobulin chains therein, and inserting into the mouse genome large fragments of human genome DNA containing loci that encode human heavy and light chain proteins. Partially modified animals, which have less than the full complement of human immunoglobulin loci, are then cross-bred to obtain an animal having all of the desired immune system modifications. When administered an immunogen, these transgenic animals produce antibodies that are immunospecific for the immunogen but have human rather than murine amino acid sequences, including the variable regions. For further details of such methods, see, for example, WO96/33735 and WO94/02602. Additional methods relating to transgenic mice for making human antibodies are described in U.S. Pat. Nos. 5,545,807; 6,713,610; 6,673,986; 6,162,963; 5,939,598; 5,545,807; 6,300,129; 6,255,458; 5,877,397; 5,874,299 and 5,545,806; in PCT publications WO91/10741, WO90/04036, WO 94/02602, WO 96/30498, WO 98/24893 and in EP 546073B1 and EP 546073A1.

The transgenic mice described above, referred to herein as “HuMab” mice, contain a human immunoglobulin gene minilocus that encodes unrearranged human heavy (mu and gamma) and kappa light chain immunoglobulin sequences, together with targeted mutations that inactivate the endogenous mu and kappa chain loci (Lonberg et al., 1994, Nature 368:856-859). Accordingly, the mice exhibit reduced expression of mouse IgM or kappa and in response to immunization, and the introduced human heavy and light chain transgenes undergo class switching and somatic mutation to generate high affinity human IgG kappa monoclonal antibodies (Lonberg et al., supra.; Lonberg and Huszar, 1995, Intern. Rev. Immunol. 13: 65-93; Harding and Lonberg, 1995, Ann. N.Y Acad. Sci. 764:536-546). The preparation of HuMab mice is described in detail in Taylor et al., 1992, Nucleic Acids Research 20:6287-6295; Chen et al., 1993, International Immunology 5:647-656; Tuaillon et al., 1994, J. Immunol. 152:2912-2920; Lonberg et al., 1994, Nature 368:856-859; Lonberg, 1994, Handbook of Exp. Pharmacology 113:49-101; Taylor et al., 1994, International Immunology 6:579-591; Lonberg and Huszar, 1995, Intern. Rev. Immunol. 13:65-93; Harding and Lonberg, 1995, Ann. N.Y Acad. Sci. 764:536-546; Fishwild et al., 1996, Nature Biotechnology 14:845-851; the foregoing references are hereby incorporated by reference in their entirety for all purposes. See, further U.S. Pat. Nos. 5,545,806; 5,569,825; 5,625,126; 5,633,425; 5,789,650; 5,877,397; 5,661,016; 5,814,318; 5,874,299; and 5,770,429; as well as U.S. Pat. No. 5,545,807; International Publication Nos. WO 93/1227; WO 92/22646; and WO 92/03918, the disclosures of all of which are hereby incorporated by reference in their entirety for all purposes. Technologies utilized for producing human antibodies in these transgenic mice are disclosed also in WO 98/24893, and Mendez et al., 1997, Nature Genetics 15:146-156, which are hereby incorporated by reference.

Human-derived antibodies can also be generated using phage display techniques. Phage display is described in e.g., Dower et al., WO 91/17271, McCafferty et al., WO 92/01047, and Caton and Koprowski, Proc. Natl. Acad. Sci. USA, 87:6450-6454 (1990), each of which is incorporated herein by reference in its entirety. The antibodies produced by phage technology are usually produced as antigen binding fragments, e.g. Fv or Fab fragments, in bacteria and thus lack effector functions. Effector functions can be introduced by one of two strategies: The fragments can be engineered either into complete antibodies for expression in mammalian cells, or into multispecific antibody fragments with a second binding site capable of triggering an effector function, if desired. Typically, the Fd fragment (VH-CH1) and light chain (VL-CL) of antibodies are separately cloned by PCR and recombined randomly in combinatorial phage display libraries, which can then be selected for binding to a particular antigen. The antibody fragments are expressed on the phage surface, and selection of Fv or Fab (and therefore the phage containing the DNA encoding the antibody fragment) by antigen binding is accomplished through several rounds of antigen binding and re-amplification, a procedure termed panning. Antibody fragments specific for the antigen are enriched and finally isolated. Phage display techniques can also be used in an approach for the humanization of rodent monoclonal antibodies, called “guided selection” (see Jespers, L. S., et al., Bio/Technology 12, 899-903 (1994)). For this, the Fd fragment of the mouse monoclonal antibody can be displayed in combination with a human light chain library, and the resulting hybrid Fab library may then be selected with antigen. The mouse Fd fragment thereby provides a template to guide the selection. Subsequently, the selected human light chains are combined with a human Fd fragment library. Selection of the resulting library yields entirely human Fab.

In certain embodiments, the multispecific antigen binding proteins of the invention are antibodies. As used herein, the term “antibody” refers to a tetrameric immunoglobulin protein comprising two light chain polypeptides (about 25 kDa each) and two heavy chain polypeptides (about 50-70 kDa each). The term “light chain” or “immunoglobulin light chain” refers to a polypeptide comprising, from amino terminus to carboxyl terminus, a single immunoglobulin light chain variable region (VL) and a single immunoglobulin light chain constant domain (CL). The immunoglobulin light chain constant domain (CL) can be kappa (κ) or lambda (λ). The term “heavy chain” or “immunoglobulin heavy chain” refers to a polypeptide comprising, from amino terminus to carboxyl terminus, a single immunoglobulin heavy chain variable region (VH), an immunoglobulin heavy chain constant domain 1 (CH1), an immunoglobulin hinge region, an immunoglobulin heavy chain constant domain 2 (CH2), an immunoglobulin heavy chain constant domain 3 (CH3), and optionally an immunoglobulin heavy chain constant domain 4 (CH4). Heavy chains are classified as mu (μ), delta (Δ), gamma (γ), alpha (α), and epsilon (ε), and define the antibody's isotype as IgM, IgD, IgG, IgA, and IgE, respectively. The IgG-class and IgA-class antibodies are further divided into subclasses, namely, IgG1, IgG2, IgG3, and IgG4, and IgA1 and IgA2, respectively. The heavy chains in IgG, IgA, and IgD antibodies have three domains (CH1, CH2, and CH3), whereas the heavy chains in IgM and IgE antibodies have four domains (CH1, CH2, CH3, and CH4). The immunoglobulin heavy chain constant domains can be from any immunoglobulin isotype, including subtypes. The antibody chains are linked together via inter-polypeptide disulfide bonds between the CL domain and the CH1 domain (i.e. between the light and heavy chain) and between the hinge regions of the antibody heavy chains.

In particular embodiments, the multispecific antigen binding proteins of the invention are heterodimeric antibodies (used interchangeably herein with “hetero immunoglobulins” or “hetero Igs”), which refer to antibodies comprising two different light chains and two different heavy chains.

The heterodimeric antibodies can comprise any immunoglobulin constant region. The term “constant region” as used herein refers to all domains of an antibody other than the variable region. The constant region is not involved directly in binding of an antigen, but exhibits various effector functions. As described above, antibodies are divided into particular isotypes (IgA, IgD, IgE, IgG, and IgM) and subtypes (IgG1, IgG2, IgG3, IgG4, IgA1 IgA2) depending on the amino acid sequence of the constant region of their heavy chains. The light chain constant region can be, for example, a kappa- or lambda-type light chain constant region, e.g., a human kappa- or lambda-type light chain constant region, which are found in all five antibody isotypes.

The heavy chain constant region of the heterodimeric antibodies can be, for example, an alpha-, delta-, epsilon-, gamma-, or mu-type heavy chain constant region, e.g., a human alpha-, delta-, epsilon-, gamma-, or mu-type heavy chain constant region. In some embodiments, the heterodimeric antibodies comprise a heavy chain constant region from an IgG1, IgG2, IgG3, or IgG4 immunoglobulin. In one embodiment, the heterodimeric antibody comprises a heavy chain constant region from a human IgG1 immunoglobulin. In another embodiment, the heterodimeric antibody comprises a heavy chain constant region from a human IgG2 immunoglobulin.

The term “antibody construct” refers to a molecule in which the structure and/or function is/are based on the structure and/or function of an antibody, e.g., of a full-length or whole immunoglobulin molecule. An antibody construct is hence capable of binding to its specific target or antigen. Furthermore, an antibody construct according to the invention comprises the minimum structural requirements of an antibody which allow for the target binding. This minimum requirement may e.g. be defined by the presence of at least the three light chain CDRs (i.e. CDR1, CDR2 and CDR3 of the VL region) and/or the three heavy chain CDRs (i.e. CDR1, CDR2 and CDR3 of the VH region), preferably of all six CDRs. The antibodies on which the constructs according to the invention are based include for example monoclonal, recombinant, chimeric, deimmunized, humanized and human antibodies. A “multispecific antibody construct” is an antibody construct that can bind more than one antigen, or bind multiple epitopes on a single antigen.

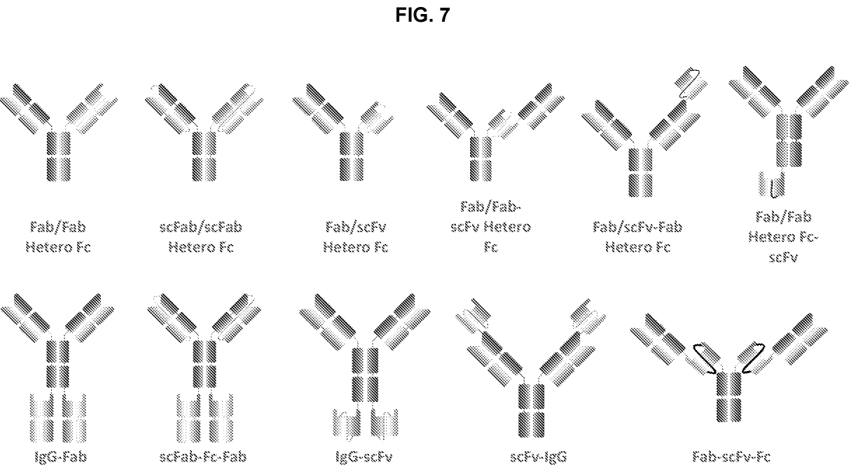

An “antibody construct module” refers to the overall design of an antibody construct and can include fragments of full-length antibodies, such as VH, VHH, VL, (s)dAb, Fv, Fd, Fab, Fab′, F(ab′)2 or “r IgG” (“half antibody”). Antibody construct modules according to the invention may also be modified fragments of antibodies, also called antibody variants, such as scFv, di-scFv or bi(s)-scFv, scFv-Fc, scFv-zipper, scFab, Fab2, Fab3, diabodies, single chain diabodies, tandem diabodies (Tandab's), tandem di-scFv, tandem tri-scFv, “minibodies” exemplified by a structure which is as follows: (VH-VL-CH3)2, (scFv-CH3)2, ((scFv)2-CH3+CH3), ((scFv)2-CH3) or (scFv-CH3-scFv)2, multibodies such as triabodies or tetrabodies, and single domain antibodies such as nanobodies or single variable domain antibodies comprising merely one variable domain, which might be VHH, VH or VL, that specifically bind an antigen or epitope independently of other V regions or domains. “Multispecific antibody construct modules” are antibody construct modules that bind more than one antigen, or bind multiple epitopes on a single antigen. Non-limiting examples are shown in FIG. 7.

In one embodiment, the multispecific antibody construct modules include at least two of the modules selected from the group consisting of Fab/Fab hetero Fc, scFab/scFab hetero Fc, Fab/scFv hetero Fc, Fab/Fab-scFv hetero Fc, Fab/scFv-Fab hetero Fc, Fab/Fab hetero Fc-scFv, IgG-Fab, scFab-Fc-Fab, IgG-scFv, scFv-IgG, and Fab-scFv-Fc.

For clarity, a “multispecific antibody construct” refers to one particular molecule while a “multispecific antibody construct module” refers to the overall design of a multispecific antibody construct. E.g., where each antibody construct element is located in relationship to each other and to an optional Fc region. Accordingly, in certain embodiments, the multispecific antibody construct modules of the present invention comprise an Fc portion. Such an Fc portion may be homodimeric or heterodimeric. Such an Fc portion may also be a single chain Fc (“scFc”) or instead be formed by two separate polypeptides.

In one embodiment, a multispecific antibody of this disclosure is a Duobody™ Duobodies can be made by the DuoBody™ technology platform (Genmab A/S) as described, e.g., in International Publication Nos. WO 2008/119353, WO 2011/131746, WO 2011/147986, and WO 2013/060867, Labrijn A F et al., PNAS, 110(13): 5145-5150 (2013), Gramer et al., mAbs, 5(6): 962-973 (2013), and Labrijn et al., Nature Protocols, 9(10): 2450-2463 (2014). This technology can be used to combine one half of a first monospecific antibody containing two heavy and two light chains with one half of a second monospecific antibody containing two heavy and two light chains. The resultant heterodimer contains one heavy chain and one light chain from the first antibody paired with one heavy chain and one light chain from the second antibody. When both of the monospecific antibodies recognize different epitopes on different antigens, the resultant heterodimer is a multispecific antibody.

For the DuoBody™ platform, each of the monospecific antibodies includes a heavy chain constant region with a single point mutation in the heavy chain. These point mutations permit a stronger interaction between the heavy chains in the resulting multispecific antibody than between the heavy chains in either of the monospecific antibodies without the mutations. The single point mutation in each monospecific antibody can be at residue 366, 368, 370, 399, 405, 407, or 409 (EU numbering) in the heavy chain of the heavy chain constant region (see, WO 2011/131746). Furthermore, the single point mutation is located at a different residue in one monospecific antibody relative to the other monospecific antibody. For example, one monospecific antibody can comprise the mutation F405L (EU numbering; phenylalanine to leucine mutation at residue 405), or one of F405A, F405D, F405E, F405H, F4051, F405K, F405M, F405N, F405Q, F405S, F405T, F405V, F405W, and F405Y mutations, while the other monospecific antibody can comprise the mutation K409R (EU numbering; lysine to arginine mutation at residue 409). The heavy chain constant regions of the monospecific antibodies can be an IgG1, IgG2, IgG3, or IgG4 isotype (e.g., a human IgG1 isotype), and a multispecific antibody produced by the DuoBody” ™ technology can be modified to alter (e.g., reduce) Fc-mediated effector functions and/or improve half-life. One method of generating a Duobody™ involves the following: (i) separate expression of two parental IgG1s containing single matching point mutations (i.e., K409R and F405L (or one of F405A, F405D, F405E, F405H, F4051, F405K, F405M, F405N, F405Q, F405S, F405T, F405V, F405W, and F405Y mutations) (EU numbering)) in the heavy chain; (ii) mixing of parental IgG1s under permissive redox conditions in vitro to enable recombination of half-molecules; (iii) removal of the reductant to allow re-oxidation of interchain disulfide bonds; and (iv) analysis of exchange efficiency and final product using chromatography-based or mass spectrometry (MS)-based methods (see, Labrijn et al., Nature Protocols, 9(10): 2450-2463 (2014)).

Another exemplary method of generating multispecific antibodies is by the knobs-into-holes technology (Ridgway et al., Protein Eng., 9:617-621 (1996); WO 2006/028936). The mispairing problem of Ig heavy chains that is a chief drawback for making multispecific antibodies is reduced in this technology by mutating selected amino acids forming the interface of the heavy chains in IgG. At positions within the heavy chain at which the two heavy chains interact directly, an amino acid with a small side chain (hole) is introduced into the sequence of one heavy chain and an amino acid with a large side chain (knob) into the counterpart interacting residue location on the other heavy chain. In some instances, antibodies of the disclosure have immunoglobulin chains in which the heavy chains have been modified by mutating selected amino acids that interact at the interface between two polypeptides so as to preferentially form a multispecific antibody. The multispecific antibodies can be composed of immunoglobulin chains of the same subclass or different subclasses. In one instance, a multispecific antibody that binds to gp120 and CD3 comprises a T366W (EU numbering) mutation in the “knobs chain” and T366S, L368A, Y407V 9EU numbering) mutations in the “hole chain.” In certain embodiments, an additional interchain disulfide bridge is introduced between the heavy chains by, e.g., introducing a Y349C mutation into the “knobs chain” and a E356C mutation or a S354C mutation into the “hole chain.” In certain embodiments, R409D, K370E mutations are introduced in the “knobs chain” and D399K, E357K mutations in the “hole chain.” In other embodiments, Y349C, T366W mutations are introduced in one of the chains and E356C, T366S, L368A, Y407V mutations in the counterpart chain. In some embodiments. Y349C, T366W mutations are introduced in one chain and S354C, T366S, L368A, Y407V mutations in the counterpart chain. In some embodiments, Y349C, T366W mutations are introduced in one chain and S354C, T366S, L368A, Y407V mutations in the counterpart chain. In yet other embodiments, Y349C, T366W mutations are introduced in one chain and S354C, T366S, L368A, Y407V mutations in the counterpart chain (all EU numbering).

Yet another method of generating multispecific antibodies is the CrossMab technology. CrossMab are chimeric antibodies constituted by the halves of two full-length antibodies. For correct chain pairing, it combines two technologies: (i) the knob-into-hole which favors a correct pairing between the two heavy chains; and (ii) an exchange between the heavy and light chains of one of the two Fabs to introduce an asymmetry which avoids light-chain mispairing. See, Ridgway et al., Protein Eng., 9:617-621 (1996); Schaefer et al., PNAS, 108:11187-11192 (2011). CrossMabs can combine two or more antigen-binding domains for targeting two or more targets or for introducing bivalency towards one target such as the 2:1 format.

To facilitate the association of a particular heavy chain with its cognate light chain, both the heavy and light chains may contain complimentary amino acid substitutions. As used herein, “complimentary amino acid substitutions” refer to a substitution to a positively-charged amino acid in one chain paired with a negatively-charged amino acid substitution in the other chain. For example, in some embodiments, the heavy chain comprises at least one amino acid substitution to introduce a charged amino acid and the corresponding light chain comprises at least one amino acid substitution to introduce a charged amino acid, wherein the charged amino acid introduced into the heavy chain has the opposite charge of the amino acid introduced into the light chain. In certain embodiments, one or more positively-charged residues (e.g., lysine, histidine or arginine) can be introduced into a first light chain (LC1) and one or more negatively-charged residues (e.g., aspartic acid or glutamic acid) can be introduced into the companion heavy chain (HC1) at the binding interface of LC1/HC1, whereas one or more negatively-charged residues (e.g., aspartic acid or glutamic acid) can be introduced into a second light chain (LC2) and one or more positively-charged residues (e.g., lysine, histidine or arginine) can be introduced into the companion heavy chain (HC2) at the binding interface of LC2/HC2. The electrostatic interactions will direct the LC1 to pair with HC1 and LC2 to pair with HC2, as the opposite charged residues (polarity) at the interface attract. The heavy/light chain pairs having the same charged residues (polarity) at an interface (e.g. LC1/HC2 and LC2/HC1) will repel, resulting in suppression of the unwanted HC/LC pairings.

In these and other embodiments, the CH1 domain of the heavy chain or the CL domain of the light chain comprises an amino acid sequence differing from wild-type IgG amino acid sequence such that one or more positively-charged amino acids in wild-type IgG amino acid sequence is replaced with one or more negatively-charged amino acids. Alternatively, the CH1 domain of the heavy chain or the CL domain of the light chain comprises an amino acid sequence differing from wild-type IgG amino acid sequence such that one or more negatively-charged amino acids in wild-type IgG amino acid sequence is replaced with one or more positively-charged amino acids. In some embodiments, one or more amino acids in the CH1 domain of the first and/or second heavy chain in the heterodimeric antibody at an EU position selected from F126, P127, L128, A141, L145, K147, D148, H168, F170, P171, V173, Q175, S176, S183, V185 and K213 is replaced with a charged amino acid. In certain embodiments, a preferred residue for substitution with a negatively- or positively-charged amino acid is 5183 (EU numbering system). In some embodiments, 5183 is substituted with a positively-charged amino acid. In alternative embodiments, 5183 is substituted with a negatively-charged amino acid. For instance, in one embodiment, S183 is substituted with a negatively-charged amino acid (e.g. S183E) in the first heavy chain, and 5183 is substituted with a positively-charged amino acid (e.g. S183K) in the second heavy chain.

In embodiments in which the light chain is a kappa light chain, one or more amino acids in the CL domain of the first and/or second light chain in the heterodimeric antibody at a position (EU and Kabat numbering in a kappa light chain) selected from F116, F118, S121, D122, E123, Q124, S131, V133, L135, N137, N138, Q160, 5162, T164, S174 and S176 is replaced with a charged amino acid. In embodiments in which the light chain is a lambda light chain, one or more amino acids in the CL domain of the first and/or second light chain in the heterodimeric antibody at a position (Kabat numbering in a lambda chain) selected from T116, F118, S121, E123, E124, K129, T131, V133, L135, S137, E160, T162, S165, Q167, A174, S176 and Y178 is replaced with a charged amino acid. In some embodiments, a preferred residue for substitution with a negatively- or positively-charged amino acid is S176 (EU and Kabat numbering system) of the CL domain of either a kappa or lambda light chain. In certain embodiments, S176 of the CL domain is replaced with a positively-charged amino acid. In alternative embodiments, S176 of the CL domain is replaced with a negatively-charged amino acid. In one embodiment, S176 is substituted with a positively-charged amino acid (e.g. S176K) in the first light chain, and S176 is substituted with a negatively-charged amino acid (e.g. S176E) in the second light chain.

In addition to or as an alternative to the complimentary amino acid substitutions in the CH1 and CL domains, the variable regions of the light and heavy chains in the heterodimeric antibody may contain one or more complimentary amino acid substitutions to introduce charged amino acids. For instance, in some embodiments, the VH region of the heavy chain or the VL region of the light chain of a heterodimeric antibody comprises an amino acid sequence differing from wild-type IgG amino acid sequence such that one or more positively-charged amino acids in wild-type IgG amino acid sequence is replaced with one or more negatively-charged amino acids. Alternatively, the VH region of the heavy chain or the VL region of the light chain comprises an amino acid sequence differing from wild-type IgG amino acid sequence such that one or more negatively-charged amino acids in wild-type IgG amino acid sequence is replaced with one or more positively-charged amino acids.

V region interface residues (i.e., amino acid residues that mediate assembly of the VH and VL regions) within the VH region include Kabat positions 1, 3, 35, 37, 39, 43, 44, 45, 46, 47, 50, 59, 89, 91, and 93. One or more of these interface residues in the VH region can be substituted with a charged (positively- or negatively-charged) amino acid. In certain embodiments, the amino acid at Kabat position 39 in the VH region of the first and/or second heavy chain is substituted for a positively-charged amino acid, e.g., lysine. In alternative embodiments, the amino acid at Kabat position 39 in the VH region of the first and/or second heavy chain is substituted for a negatively-charged amino acid, e.g., glutamic acid. In some embodiments, the amino acid at Kabat position 39 in the VH region of the first heavy chain is substituted for a negatively-charged amino acid (e.g. G39E), and the amino acid at Kabat position 39 in the VH region of the second heavy chain is substituted for a positively-charged amino acid (e.g. G39K). In some embodiments, the amino acid at Kabat position 44 in the VH region of the first and/or second heavy chain is substituted for a positively-charged amino acid, e.g., lysine. In alternative embodiments, the amino acid at Kabat position 44 in the VH region of the first and/or second heavy chain is substituted for a negatively-charged amino acid, e.g., glutamic acid. In certain embodiments, the amino acid at Kabat position 44 in the VH region of the first heavy chain is substituted for a negatively-charged amino acid (e.g. G44E), and the amino acid at Kabat position 44 in the VH region of the second heavy chain is substituted for a positively-charged amino acid (e.g. G44K).

V region interface residues (i.e., amino acid residues that mediate assembly of the VH and VL regions) within the VL region include Kabat positions 32, 34, 35, 36, 38, 41, 42, 43, 44, 45, 46, 48, 49, 50, 51, 53, 54, 55, 56, 57, 58, 85, 87, 89, 90, 91, and 100. One or more interface residues in the VL region can be substituted with a charged amino acid, preferably an amino acid that has an opposite charge to those introduced into the VH region of the cognate heavy chain. In some embodiments, the amino acid at Kabat position 100 in the VL region of the first and/or second light chain is substituted for a positively-charged amino acid, e.g., lysine. In alternative embodiments, the amino acid at Kabat position 100 in the VL region of the first and/or second light chain is substituted for a negative-charged amino acid, e.g., glutamic acid. In certain embodiments, the amino acid at Kabat position 100 in the VL region of the first light chain is substituted for a positively-charged amino acid (e.g. G100K), and the amino acid at Kabat position 100 in the VL region of the second light chain is substituted for a negatively-charged amino acid (e.g. G100E).

In one embodiment, a first Fc region comprises negatively charged amino acids at residues corresponding to positions 409 and 392 and a second Fc region comprises positively charged amino acids at residues corresponding to positions 399 and 356, wherein the numbering of amino acid residues is according to the EU index as set forth in Kabat.

In one embodiment, the first Fc region comprises K/R409D and K392D mutations and the second Fc region comprises D399K and E356K mutations, wherein the numbering of amino acid residues is according to the EU index as set forth in Kabat.

In one embodiment, a first Fc region comprises negatively charged amino acids at residues corresponding to positions 409, 439, and 392 and a second Fc region comprises positively charged amino acids at residues corresponding to positions 399 and 356, wherein the numbering of amino acid residues is according to the EU index as set forth in Kabat.

In one embodiment, the first Fc region comprises K/R409D, K439D, and K392D mutations and the second Fc region comprises D399K and E356K mutations, wherein the numbering of amino acid residues is according to the EU index as set forth in Kabat.

In one embodiment, one Fc region comprises a F405L, F405A, F405D, F405E, F405H, F405I, F405K, F405M, F405N, F405Q, F405S, F405T, F405V, F405W, or F405Y mutation; and the other Fc region comprises a K409R mutation; wherein the numbering of amino acid residues is according to the EU index as set forth in Kabat. In one embodiment, one Fc region comprises a T366W mutation; and the other Fc region comprises T366S, L368A, Y407V mutations; wherein the numbering of amino acid residues is according to the EU index as set forth in Kabat. In one embodiment, one Fc region comprises K/R409D and K370E mutations; and the other Fc region comprises D399K and E357K mutations; wherein the numbering of amino acid residues is according to the EU index as set forth in Kabat.

In particular embodiments, the heterodimeric antibody comprises a first Fc region comprising negatively-charged amino acids at residues corresponding to positions 392 and 409 (e.g., K392D and K409D substitutions), and a second Fc region comprising positively-charged amino acids at residues corresponding to positions 356 and 399 (e.g., E356K and D399K substitutions). In other particular embodiments, the heterodimeric antibody comprises a first Fc region comprising negatively-charged amino acids at residues corresponding to positions 392, 409, and 439 (e.g., K392D, K409D, and K439D substitutions), and a second Fc region comprising positively-charged amino acids at residues corresponding to positions 356 and 399 (e.g., E356K and D399K substitutions). In other particular embodiments, the heterodimeric antibody comprises a first Fc region comprising negatively-charged amino acids at residues corresponding to positions 392, 409, and 370 (e.g., K392D, K409D, and K370D substitutions), and a second Fc region comprising positively-charged amino acids at residues corresponding to positions 356, 399, and 357 (e.g., E356K, D399K, and E357K substitutions).

In one embodiment, one Fc region comprises a Y349C mutation; and the other Fc region comprises either a E356C or a S354C mutation; wherein the numbering of amino acid residues is according to the EU index as set forth in Kabat. In one embodiment, one Fc region comprises Y349C and T366W mutations; and the other Fc region comprises E356C, T366S, L368A, and Y407V mutations; wherein the numbering of amino acid residues is according to the EU index as set forth in Kabat. In one embodiment, one Fc region comprises Y349C and T366W mutations; and the other Fc region comprises S354C, T366S, L368A, Y407V mutations; wherein the numbering of amino acid residues is according to the EU index as set forth in Kabat.

In certain embodiments, a heterodimeric antibody of the invention comprises a first heavy chain and a second heavy chain and a first light chain and a second light chain, wherein the first heavy chain comprises amino acid substitutions at positions 183 (EU), 392 (EU) and 409 (EU), wherein the second heavy chain comprises amino acid substitutions at positions 183 (EU), 356 (EU) and 399 (EU), wherein the first and second light chains comprise an amino acid substitution at position 176 (EU), and wherein the amino acid substitutions introduce a charged amino acid at said positions. In related embodiments, the serine at position 176 (EU) of the first light chain is replaced with lysine, the serine at position 176 (EU) of the second light chain is replaced with glutamic acid, the serine at position 183 (EU) of the first heavy chain is replaced with glutamic acid, the lysine at position 392 (EU) of the first heavy chain is replaced with aspartic acid, the lysine at position 409 (EU) of the first heavy chain is replaced with aspartic acid, the serine at position 183 (EU) of the second heavy chain is replaced with lysine, the glutamic acid at position 356 (EU) of the second heavy chain is replaced with lysine, and/or the aspartic acid at position 399 (EU) of the second heavy chain is replaced with lysine.

In certain embodiments, a heterodimeric antibody of the invention comprises a first heavy chain and a second heavy chain and a first light chain and a second light chain, wherein the first heavy chain comprises amino acid substitutions at positions 183 (EU), 392 (EU), 409 (EU), and 439 (EU) wherein the second heavy chain comprises amino acid substitutions at positions 183 (EU), 356 (EU) and 399 (EU), wherein the first and second light chains comprise an amino acid substitution at position 176 (EU), and wherein the amino acid substitutions introduce a charged amino acid at said positions. In related embodiments, the serine at position 176 (EU) of the first light chain is replaced with lysine, the serine at position 176 (EU) of the second light chain is replaced with glutamic acid, the serine at position 183 (EU) of the first heavy chain is replaced with glutamic acid, the lysine at position 392 (EU) of the first heavy chain is replaced with aspartic acid, the lysine at position 409 (EU) of the first heavy chain is replaced with aspartic acid, the lysine at position 439 (EU) of the first heavy chain is replaced with aspartic acid, the serine at position 183 (EU) of the second heavy chain is replaced with lysine, the glutamic acid at position 356 (EU) of the second heavy chain is replaced with lysine, and/or the aspartic acid at position 399 (EU) of the second heavy chain is replaced with lysine.

In certain embodiments, a heterodimeric antibody of the invention comprises a first heavy chain and a second heavy chain and a first light chain and a second light chain, wherein the first heavy chain comprises amino acid substitutions at positions 44 (Kabat), 183 (EU), 392 (EU) and 409 (EU), wherein the second heavy chain comprises amino acid substitutions at positions 44 (Kabat), 183 (EU), 356 (EU) and 399 (EU), wherein the first and second light chains comprise an amino acid substitution at positions 100 (Kabat) and 176 (EU), and wherein the amino acid substitutions introduce a charged amino acid at said positions. In related embodiments, the glycine at position 44 (Kabat) of the first heavy chain is replaced with glutamic acid, the glycine at position 44 (Kabat) of the second heavy chain is replaced with lysine, the glycine at position 100 (Kabat) of the first light chain is replaced with lysine, the glycine at position 100 (Kabat) of the second light chain is replaced with glutamic acid, the serine at position 176 (EU) of the first light chain is replaced with lysine, the serine at position 176 (EU) of the second light chain is replaced with glutamic acid, the serine at position 183 (EU) of the first heavy chain is replaced with glutamic acid, the lysine at position 392 (EU) of the first heavy chain is replaced with aspartic acid, the lysine at position 409 (EU) of the first heavy chain is replaced with aspartic acid, the serine at position 183 (EU) of the second heavy chain is replaced with lysine, the glutamic acid at position 356 (EU) of the second heavy chain is replaced with lysine, and/or the aspartic acid at position 399 (EU) of the second heavy chain is replaced with lysine.

As used herein, the term “Fc region” refers to the C-terminal region of an immunoglobulin heavy chain which may be generated by papain digestion of an intact antibody. The Fc region of an immunoglobulin generally comprises two constant domains, a CH2 domain and a CH3 domain, and optionally comprises a CH4 domain. In certain embodiments, the Fc region is an Fc region from an IgG1, IgG2, IgG3, or IgG4 immunoglobulin. In some embodiments, the Fc region comprises CH2 and CH3 domains from a human IgG1 or human IgG2 immunoglobulin. The Fc region may retain effector function, such as Clq binding, complement dependent cytotoxicity (CDC), Fc receptor binding, antibody-dependent cell-mediated cytotoxicity (ADCC), and phagocytosis. In other embodiments, the Fc region may be modified to reduce or eliminate effector function as described in further detail herein.

In some embodiments of the antigen binding proteins of the invention, the binding domain positioned at the carboxyl terminus of the Fc region (i.e. the carboxyl-terminal binding domain) is a scFv. In certain embodiments, the scFv comprises a heavy chain variable region (VH) and light chain variable region (VL) connected by a peptide linker. The variable regions may be oriented within the scFv in a VH-VL or VL-VH orientation. For instance, in one embodiment, the scFv comprises, from N-terminus to C-terminus, a VH region, a peptide linker, and a VL region. In another embodiment, the scFv comprises, from N-terminus to C-terminus, a VL region, a peptide linker, and a VH region. The VH and VL regions of the scFv may contain one or more cysteine substitutions to permit disulfide bond formation between the VH and VL regions. Such cysteine clamps stabilize the two variable domains in the antigen-binding configuration. In one embodiment, position 44 (Kabat numbering) in the VH region and position 100 (Kabat numbering) in the VL region are each substituted with a cysteine residue.

In one aspect the present invention is directed to a method for selecting a multispecific antibody construct, the method comprising:
(a) obtaining a plurality of antibody Fab fragments, scFvs, or a combination thereof, wherein each Fab fragment and scFv comprises three heavy chain CDRs and three light chain CDRs that specifically bind a first antigen;
(b) obtaining a plurality of antibody Fab fragments, scFvs, or a combination thereof, wherein each Fab fragment and scFv comprises three heavy chain CDRs and three light chain CDRs that specifically bind a second antigen;
(c) cloning into a vector:
(i) the CDRs of the three heavy chain CDRs that specifically bind the first antigen,
(ii) the CDRs of the three heavy chain CDRs that specifically bind the second antigen, and
(iii) three light chain CDRs selected from the group consisting of
(a) the CDRs of the three light chain CDRs that specifically bind the first antigen,
(b) the CDRs of the three light chain CDRs that specifically bind the second antigen, and
(c) CDRs of three light chain CDRs that do not specifically bind either the first antigen or the second antigen;
wherein the vector(s) encode(s) for one type of multispecific antibody construct module and a plurality of vectors are generated that encode for a plurality of multispecific antibody constructs comprising the heavy chain CDRs that bind to each antigen and the light chain CDRs of (c)(iii);
(d) expressing each multispecific antibody construct in a mammalian host cell;
(e) purifying each multispecific antibody construct;
(f)(i) measuring the expression levels of each multispecific antibody construct and (ii) measuring the binding affinity of each multispecific antibody construct to the first antigen and the second antigen,
wherein steps (f)(i) and (f)(ii) can be performed simultaneously or in any order; and
(g) comparing the expression levels of (f)(i) and the binding affinities of (f)(ii) for each multispecific antibody construct in order to identify the optimal pairing of the three heavy chain CDRs that specifically bind a first antigen and the light chain CDRs of (c)(iii) with the optimal pairing of the three heavy chain CDRs that specifically bind a second antigen and the same light chain CDRs of (c)(iii).

The CDRs can be cloned into the same vector or into different vectors, as long as the multiple vectors are transfected into the same mammalian cell. Cloning the CDRs into vectors includes inserting the CDRHs into a VH framework and the CDRLs into a VL framework. The VHs and VLs thus formed can then be fused to each other via a linker, to form a scFv, or they can be fused to CH1 and CL constant regions.

The vectors generated are then transfected into a mammalian host cell wherein one type of multispecific antibody construct module is expressed. However, many different types of multispecific antibody constructs of this particular module are produced due to the plurality of Fab and/or scFv fragments from which they are formed.

Using the method described above, common light chains that can bind to either antigen are identified. The source of these light chains can be either the three light chain CDRs that specifically bind the first antigen, the three light chain CDRs that specifically bind the second antigen, or even CDRs that come from Fab and/or scFv fragments that were identified by binding to a completely different third antigen. In this case, the heavy chain CDRs would do the bulk of the binding to the antigen and the light chain CDRs would have a more passive role.

In one aspect the present invention is directed to a method for selecting a multispecific antibody construct, the method comprising:
(a) obtaining a plurality of antibody Fab fragments, scFvs, or a combination thereof, wherein each Fab fragment and scFv comprises three heavy chain CDRs and three light chain CDRs that specifically bind a first antigen;
(b) obtaining a plurality of antibody Fab fragments, scFvs, or a combination thereof, wherein each Fab fragment and scFv comprises three heavy chain CDRs and three light chain CDRs that specifically bind a second antigen;
(c) cloning the CDRs of the two pluralities into a vector(s) that encode(s) for a multispecific antibody construct module and a plurality of vectors are generated that encode a plurality of multispecific antibody constructs comprising the CDRs that bind to each antigen;
(d) expressing each multispecific antibody construct in a mammalian host cell;
(e) purifying each multispecific antibody construct;
(f)(i) measuring the expression levels of each multispecific antibody construct and (ii) calculating the percent of correct and incorrect multispecific antibody construct module species produced,
wherein steps (f)(i) and (f)(ii) can be performed simultaneously or in any order; and
(g) comparing the expression levels of (f)(i) and the percentage of correct and incorrect multispecific antibody construct module species of (f)(ii) for each multispecific antibody construct in order to identify the optimal pairing of three heavy chain CDRs and three light chain CDRs that specifically bind a first antigen with three heavy chain CDRs and three light chain CDRs that specifically bind a second antigen.

The CDRs can be cloned into the same vector or into different vectors, as long as the multiple vectors are transfected into the same mammalian cell. Cloning the CDRs into vectors includes inserting the CDRHs into a VH framework and the CDRLs into a VL framework. The VHs and VLs thus formed can then be fused to each other via a linker, to form a scFv, or they can be fused to CH1 and CL constant regions.

The vectors generated are then transfected into a mammalian host cell wherein one type of multispecific antibody construct module is expressed. However, many different types of multispecific antibody constructs of this particular module are produced due to the plurality of Fab and/or scFv fragments from which they are formed.

Using the method described above, optimal binding pairs that bind each antigen in a particular module are identified.

In one embodiment, the plurality of antibody Fab fragments, scFvs, or a combination thereof, wherein each Fab fragment and scFv comprise three heavy chain CDRs and three light chain CDRs that specifically bind a first antigen is selected from the group consisting of at least two antibody Fab fragments, scFvs, or a combination thereof; at least three antibody Fab fragments, scFvs, or a combination thereof; at least four antibody Fab fragments, scFvs, or a combination thereof; at least five antibody Fab fragments, scFvs, or a combination thereof; at least six antibody Fab fragments, scFvs, or a combination thereof; at least seven antibody Fab fragments, scFvs, or a combination thereof; at least eight antibody Fab fragments, scFvs, or a combination thereof; at least nine antibody Fab fragments, scFvs, or a combination thereof; and at least ten antibody Fab fragments, scFvs, or a combination thereof.

In one embodiment, the plurality of antibody Fab fragments, scFvs, or a combination thereof, wherein each Fab fragment and scFv comprise three heavy chain CDRs and three light chain CDRs that specifically bind a second antigen is selected from the group consisting of at least two antibody Fab fragments, scFvs, or a combination thereof; at least three antibody Fab fragments, scFvs, or a combination thereof; at least four antibody Fab fragments, scFvs, or a combination thereof; at least five antibody Fab fragments, scFvs, or a combination thereof; at least six antibody Fab fragments, scFvs, or a combination thereof; at least seven antibody Fab fragments, scFvs, or a combination thereof; at least eight antibody Fab fragments, scFvs, or a combination thereof; at least nine antibody Fab fragments, scFvs, or a combination thereof; and at least ten antibody Fab fragments, scFvs, or a combination thereof.

In one aspect the present invention is directe to a method for selecting a multispecific antibody construct, the method comprising:
(a) obtaining a first antibody Fab fragment or scFv, wherein each Fab fragment or scFv comprises three heavy chain CDRs and three light chain CDRs that specifically bind a first antigen;
(b) obtaining a second antibody Fab fragment or scFv, wherein each Fab fragment or scFv comprises three heavy chain CDRs and three light chain CDRs that specifically bind a second antigen;
(c) cloning into a vector:
(i) the CDRs of the three heavy chain CDRs that specifically bind the first antigen,
(ii) the CDRs of the three heavy chain CDRs that specifically bind the second antigen, and
(iii) three light chain CDRs selected from the group consisting of
(a) the CDRs of the three light chain CDRs that specifically bind the first antigen,
(b) the CDRs of the three light chain CDRs that specifically bind the second antigen, and
(c) CDRs of three light chain CDRs that do not specifically either the first antigen or the second antigen;
wherein the vector(s) encode(s) for more than one type of multispecific antibody construct module and a plurality of vectors are generated that encode for a plurality of multispecific antibody constructs of different modules comprising the heavy chain CDRs that bind to each antigen and the light chain CDRs of (c)(iii);
(d) expressing each multispecific antibody construct of the different modules in a mammalian host cell;
(e) purifying each multispecific antibody construct;
(f)(i) measuring the expression levels of each multispecific antibody construct and (ii) measuring the binding affinity of each multispecific antibody construct to the first antigen and the second antigen,
wherein steps (f)(i) and (f)(ii) can be performed simultaneously or in any order; and
(g) comparing the expression levels of (f)(i) and the binding affinities of (f)(ii) for each multispecific antibody construct in order to identify the optimal module for pairing of the three heavy chain CDRs that specifically bind a first antigen and the light chain CDRs of (c)(iii) with the three heavy chain CDRs that specifically bind a second antigen and the same light chain CDRs of (c)(iii).

The CDRs can be cloned into the same vector or into different vectors, as long as the multiple vectors are transfected into the same mammalian cell. Cloning the CDRs into vectors includes inserting the CDRHs into a VH framework and the CDRLs into a VL framework. The VHs and VLs thus formed can then be fused to each other via a linker, to form a scFv, or they can be fused to CH1 and CL constant regions.

The vectors generated are then transfected into a mammalian host cell wherein many types of multispecific antibody construct modules are expressed, but the antigen binding portions have the same CDRs (6 CDRs for one antigen binding portion, 6 for the other antigen binding portion, but there can be multiple antigen biding portions in a particular module; see FIG. 7).

Using the method described above, the optimal multispecific antibody construct module for the antigen binding portions that utilize a cLC are identified.

In one aspect the present invention is directed to a method for selecting a multispecific antibody construct, the method comprising:
(a) obtaining a first antibody Fab fragment or scFv, wherein each Fab fragment or scFv comprises three heavy chain CDRs and three light chain CDRs that specifically bind a first antigen;
(b) obtaining a second antibody Fab fragment or scFv, wherein each Fab fragment or scFv comprises three heavy chain CDRs and three light chain CDRs that specifically bind a second antigen;
(c) cloning the CDRs of the first antibody Fab fragment or scFv and the second antibody Fab fragment or scFv into a vector(s),
wherein the vector(s) encode(s) for more than one type of multispecific antibody construct module and a plurality of vectors are generated that encode for a plurality of multispecific antibody constructs of different modules comprising the CDRs that bind to each antigen;
(d) expressing each multispecific antibody construct in a mammalian host cell;
(e) purifying each multispecific antibody construct;
(f)(i) measuring the expression levels of each multispecific antibody construct and (ii) calculating the percent of correct and incorrect multispecific antibody construct module species produced,
wherein steps (f)(i) and (f)(ii) can be performed simultaneously or in any order; and
(g) comparing the expression levels of (f)(i) and the percentage of correct and incorrect multispecific antibody construct module species of (f)(ii) for each multispecific antibody construct in order to identify the optimal multispecific antibody construct module for pairing of the three heavy chain CDRs and three light chain CDRs that specifically bind a first antigen with the three heavy chain CDRs and three light chain CDRs that specifically bind a second antigen.

The CDRs can be cloned into the same vector or into different vectors, as long as the multiple vectors are transfected into the same mammalian cell. Cloning the CDRs into vectors includes inserting the CDRHs into a VH framework and the CDRLs into a VL framework. The VHs and VLs thus formed can then be fused to each other via a linker, to form a scFv, or they can be fused to CH1 and CL constant regions.

The vectors generated are then transfected into a mammalian host cell wherein many types of multispecific antibody construct modules are expressed, but the antigen binding portions have the same CDRs (6 CDRs for one antigen binding portion, 6 for the other antigen binding portion, but there can be multiple antigen biding portions in a particular module; see FIG. 7).

Using the method described above, the optimal multispecific antibody construct module for the antigen binding portions are identified.

In one embodiment, the multispecific antibody construct modules include at least two of the modules selected from the group consisting of Fab/Fab hetero Fc, scFab/scFab hetero Fc, Fab/scFv hetero Fc, Fab/Fab-scFv hetero Fc, Fab/scFv-Fab hetero Fc, Fab/Fab hetero Fc-scFv, IgG-Fab, scFab-Fc-Fab, IgG-scFv, scFv-IgG, and Fab-scFv-Fc.

In one embodiment, the mammalian host cell is selected from the group consisting of Chinese hamster ovary (“CHO”) cells, monkey kidney CV1 line transformed by SV40 (“COS-7”); human embryonic kidney line 293 (“HEK293”); baby hamster kidney cells (“BHK”); mouse sertoli cells (“TM4”); monkey kidney cells (“CV1”); African green monkey kidney cells (“VERO-76”); human cervical carcinoma cells (“HELA”); canine kidney cells (“MDCK”); buffalo rat liver cells (“BRL”); human lung cells (“W138”); human hepatoma cells (“Hep G2”); mouse mammary tumor (“MMT”); TRI cells; MRC 5 cells: and FS4 cells.

In one embodiment, the expression levels are determined by a method selected from the group consisting of A280 measurement SDS-PAGE, microchip capillary electrophoresis (MCE), Bradford assay, and bicinchoninic acid (BCA) assay.

In one embodiment, the binding affinity of each multispecific antibody construct to the first antigen and the second antigen is measured using Octet, Forte Bio, Carterra LSA, SPR and Flow cytometry.

In one embodiment, the percentage of correct and incorrect multispecific antibody construct modality species is determined by a method selected from the group consisting of liquid chromatography-mass spectrometry (“LC-MS”), Caliper, HPLC SEC, SDS-PAGE, and microchip capillary electrophoresis (“MCE”).

In one embodiment, each multispecific antibody construct is purified by Protein A, Lambda and Kappa resins, and affinity tag purification. In certain embodiments, the affinity tag is selected from the group consisting of polyHis (such as hexaHis), streptavidin, FLAG, HA (hemaglutinin influenza virus), and myc tags.

In one aspect the present invention is directed to a method for selecting a multispecific antibody construct, the method comprising:
(a) obtaining a plurality of at least two antibody Fab fragments, scFvs, or a combination thereof, wherein each Fab fragment and scFv comprises three heavy chain CDRs and three light chain CDRs that specifically bind a first antigen;
(b) obtaining a plurality of at least two antibody Fab fragments, scFvs, or a combination thereof, wherein each Fab fragment and scFv comprises three heavy chain CDRs and three light chain CDRs that specifically bind a second antigen;
(c) cloning into a vector:
(i) the CDRs of the three heavy chain CDRs that specifically bind the first antigen,
(ii) the CDRs of the three heavy chain CDRs that specifically bind the second antigen, and
(iii) three light chain CDRs selected from the group consisting of
(a) the CDRs of the three light chain CDRs that specifically bind the first antigen,
(b) the CDRs of the three light chain CDRs that specifically bind the second antigen, and
(c) CDRs of three light chain CDRs that do not specifically either the first antigen or the second antigen;
wherein the vector(s) encode(s) for a multispecific antibody construct module and a plurality of vectors are generated that encode for a plurality of multispecific antibody constructs comprising the heavy chain CDRs that bind to each antigen and the light chain CDRs of (c)(iii);
(d) expressing each multispecific antibody construct in a mammalian host cell, wherein the mammalian host cell is selected from the group consisting of HEK293 cells and CHO cells;
(e) purifying each multispecific antibody construct using Protein A chromatography;
(f)(i) measuring the expression levels of each multispecific antibody construct using A280 measurement and (ii) measuring the binding affinity of each multispecific antibody construct to the first antigen and the second antigen using Octet,
wherein steps (f)(i) and (f)(ii) can be performed simultaneously or in any order; and
(g) comparing the expression levels of (f)(i) and the binding affinities of (f)(ii) for each multispecific antibody construct in order to identify the optimal pairing of the three heavy chain CDRs that specifically bind a first antigen and the light chain CDRs of (c)(iii) with the optimal pairing of the three heavy chain CDRs that specifically bind a second antigen and the same light chain CDRs of (c)(iii).

In one aspect the present invention is directed to a method for selecting a multispecific antibody construct, the method comprising:
(a) obtaining a plurality of at least two antibody Fab fragments, scFvs, or a combination thereof, wherein each Fab fragment and scFv comprises three heavy chain CDRs and three light chain CDRs that specifically bind a first antigen;
(b) obtaining a plurality of at least two antibody Fab fragments, scFvs, or a combination thereof, wherein each Fab fragment and scFv comprises three heavy chain CDRs and three light chain CDRs that specifically bind a second antigen;
(c) cloning the CDRs of the two pluralities into a vector(s) that encode(s) for a multispecific antibody construct module and a plurality of vectors are generated that encode a plurality of multispecific antibody constructs that bind to each antigen;
(d) expressing each multispecific antibody construct in a mammalian host cell, wherein the mammalian host cell is selected from the group consisting of HEK293 cells and CHO cells;
(e) purifying each multispecific antibody construct using Protein A chromatography;
(f)(i) measuring the expression levels of each multispecific antibody construct using A280 measurement and (ii) calculating the percent of correct and incorrect multispecific antibody construct module species produced using liquid chromatography-mass spectrometry (“LC-MS”),
wherein steps (f)(i) and (f)(ii) can be performed simultaneously or in any order; and
(g) comparing the expression levels of (f)(i) and the percentage of correct and incorrect multispecific antibody construct module species of (f)(ii) for each multispecific antibody construct in order to identify the optimal pairing of three heavy chain CDRs and three light chain CDRs that specifically bind a first antigen with three heavy chain CDRs and three light chain CDRs that specifically bind a second antigen.

In one aspect the present invention is directed to a method for selecting a multispecific antibody construct, the method comprising:
(a) obtaining a first antibody Fab fragment or scFv, wherein each Fab fragment or scFv comprises three heavy chain CDRs and three light chain CDRs that specifically bind a first antigen;
(b) obtaining a second antibody Fab fragment or scFv, wherein each Fab fragment or scFv comprises three heavy chain CDRs and three light chain CDRs that specifically bind a second antigen;
(c) cloning into a vector:
(i) the CDRs of the three heavy chain CDRs that specifically bind the first antigen,
(ii) the CDRs of the three heavy chain CDRs that specifically bind the second antigen, and
(iii) three light chain CDRs selected from the group consisting of
(a) the CDRs of the three light chain CDRs that specifically bind the first antigen,
(b) the CDRs of the three light chain CDRs that specifically bind the second antigen, and
(c) CDRs of three light chain CDRs that do not specifically either the first antigen or the second antigen;
wherein the vector(s) encode(s) for more than one type of multispecific antibody construct module and a plurality of vectors are generated that encode for a plurality of multispecific antibody constructs of different modules comprising the heavy chain CDRs that bind to each antigen and the light chain CDRs of (c)(iii);
(d) expressing each multispecific antibody construct in a mammalian host cell, wherein the mammalian host cell is selected from the group consisting of HEK293 cells and CHO cells;
(e) purifying each multispecific antibody construct using Protein A chromatography;
(f)(i) measuring the expression levels of each multispecific antibody construct using A280 measurement and (ii) measuring the binding affinity of each multispecific antibody construct to the first antigen and the second antigen using Octet,
(g) comparing the expression levels of (f)(i) and the binding affinities of (f)(ii) for each multispecific antibody construct in order to identify the optimal module for pairing of the three heavy chain CDRs that specifically bind a first antigen and the light chain CDRs of (c)(iii) with the three heavy chain CDRs that specifically bind a second antigen and the same light chain CDRs of (c)(iii).

In one aspect the present invention is directed to a method for selecting a multispecific antibody construct, the method comprising:
(a) obtaining a first antibody Fab fragment or scFv, wherein each Fab fragment or scFv comprises three heavy chain CDRs and three light chain CDRs that specifically bind a first antigen;
(b) obtaining a second antibody Fab fragment or scFv, wherein each Fab fragment or scFv comprises three heavy chain CDRs and three light chain CDRs that specifically bind a second antigen;
(c) cloning the CDRs of the first antibody Fab fragment or scFv and the second antibody Fab fragment or scFv into a vector(s),
wherein the vector(s) encode(s) for more than one type of multispecific antibody construct module and a plurality of vectors are generated that encode for a plurality of multispecific antibody constructs of different modules comprising the CDRs that bind to each antigen;
(d) expressing each multispecific antibody construct in a mammalian host cell, wherein the mammalian host cell is selected from the group consisting of HEK293 cells and CHO cells;
(e) purifying each multispecific antibody construct using Protein A chromatography;
(f)(i) measuring the expression levels of each multispecific antibody construct using A280 and (ii) calculating the percent of correct and incorrect multispecific antibody construct module species produced using liquid chromatography-mass spectrometry (“LC-MS”),
wherein steps (f)(i) and (f)(ii) can be performed simultaneously or in any order; and
(g) comparing the expression levels of (f)(i) and the percentage of correct and incorrect multispecific antibody construct module species of (f)(ii) for each multispecific antibody construct in order to identify the optimal multispecific antibody construct module for pairing of the three heavy chain CDRs and three light chain CDRs that specifically bind a first antigen with the three heavy chain CDRs and three light chain CDRs that specifically bind a second antigen.

In certain embodiments, the scFv is fused or otherwise connected at its amino terminus to the carboxyl terminus of the Fc region (e.g. the carboxyl terminus of the CH3 domain) through a peptide linker. Thus, in one embodiment, the scFv is fused to an Fc region such that the resulting fusion protein comprises, from N-terminus to C-terminus, a CH2 domain, a CH3 domain, a first peptide linker, a VH region, a second peptide linker, and a VL region. In another embodiment, the scFv is fused to an Fc region such that the resulting fusion protein comprises, from N-terminus to C-terminus, a CH2 domain, a CH3 domain, a first peptide linker, a VL region, a second peptide linker, and a VH region. A “fusion protein” is a protein that includes polypeptide components derived from more than one parental protein or polypeptide. Typically, a fusion protein is expressed from a fusion gene in which a nucleotide sequence encoding a polypeptide sequence from one protein is appended in frame with, and optionally separated by a linker from, a nucleotide sequence encoding a polypeptide sequence from a different protein. The fusion gene can then be expressed by a recombinant host cell to produce the single fusion protein.

A “peptide linker” refers to an oligopeptide of about 2 to about 50 amino acids that covalently joins one polypeptide to another polypeptide. The peptide linkers can be used to connect the VH and VL domains within the scFv. The peptide linkers can also be used to connect a scFv, Fab fragment, or other functional antibody fragment to the amino terminus or carboxyl terminus of an Fc region to create multispecific antigen binding proteins as described herein. Preferably, the peptide linkers are at least 5 amino acids in length. In certain embodiments, the peptide linkers are from about 5 amino acids in length to about 40 amino acids in length. In other embodiments, the peptide linkers are from about 8 amino acids in length to about 30 amino acids in length. In still other embodiments, the peptide linkers are from about 10 amino acids in length to about 20 amino acids in length.

Preferably, but not necessarily, the peptide linker comprises amino acids from among the twenty canonical amino acids, particularly cysteine, glycine, alanine, proline, asparagine, glutamine, and/or serine. In certain embodiments, the peptide linker is comprised of a majority of amino acids that are sterically unhindered, such as glycine, serine, and alanine. Thus, linkers that are preferred in some embodiments, include polyglycines, polyserines, and polyalanines, or combinations of any of these. Some exemplary peptide linkers include, but are not limited to, poly(Gly)₂-s (SEQ ID NOs: 22-26, 30, and 51), particularly (Gly)₃ (SEQ ID NO: 22), (Gly)₄(SEQ ID NO: 23), (Gly)₅ (SEQ ID NO: 24), (Gly)₆ (SEQ ID NO: 25) and (Gly)₇ (SEQ ID NO: 26), as well as, poly(Gly)₄Ser (SEQ ID NO: 48), poly(Gly-Ala)₂₋₄(SEQ ID NOs: 33-35) and poly(Ala)₂₋₈(SEQ ID NOs: 36-42). In certain embodiments, the peptide linker is (GlyₓSer)ₙ where x=3 or 4 and n=2, 3, 4, 5 or 6 (SEQ ID NOs: 29, 31, 32 and 43-50). Such peptide linkers include “L5” (GGGGS or “G₄S”; SEQ ID NO: 27), “L9” (GGGSGGGGS; or “G₃SG₄S”; SEQ ID NO: 28), “L10” (GGGGSGGGGS; or “(G₄S)2”; SEQ ID NO: 29), “L15” (GGGGSGGGGSGGGGS; or “(G₄S)₃”; SEQ ID NO: 31), and “L25” (GGGGSGGGGSGGGGSGGGGSGGGGS; or “(G₄S)₅”; SEQ ID NO:32). In some embodiments, the peptide linker joining the VH and VL regions within the scFv is a L15 or (G₄S)₃ linker (SEQ ID NO: 31). In these and other embodiments, the peptide linker joining the carboxyl-terminal binding domain (e.g. scFv or Fab) to the C-terminus of the Fc region is a L9 or G₃SG₄S linker (SEQ ID NO: 28) or a L10 (G₄S)₂ linker (SEQ ID NO: 29).

Other specific examples of peptide linkers that may be used in the multispecific antigen binding proteins of the invention include (Gly)₅Lys (SEQ ID NO: 1); (Gly)₅LysArg (SEQ ID NO: 2); (Gly)₃Lys(Gly)₄ (SEQ ID NO: 3); (Gly)₃AsnGlySer(Gly)₂ (SEQ ID NO: 4); (Gly)₃Cys(Gly)₄ (SEQ ID NO: 5); GlyProAsnGlyGly (SEQ ID NO: 6); GGEGGG (SEQ ID NO: 7); GGEEEGGG (SEQ ID NO: 8); GEEEG (SEQ ID NO: 9); GEEE (SEQ ID NO: 10); GGDGGG (SEQ ID NO: 11); GGDDDGG (SEQ ID NO: 12); GDDDG (SEQ ID NO: 13); GDDD (SEQ ID NO: 14); GGGGSDDSDEGSDGEDGGGGS (SEQ ID NO: 15); WEWEW (SEQ ID NO: 16); FEFEF (SEQ ID NO: 17); EEEWWW (SEQ ID NO: 18); EEEFFF (SEQ ID NO: 19); WWEEEWW (SEQ ID NO: 20); and FFEEEFF (SEQ ID NO: 21).

The heavy chain constant regions or the Fc regions of the multispecific antigen binding proteins described herein may comprise one or more amino acid substitutions that affect the glycosylation and/or effector function of the antigen binding protein. One of the functions of the Fc region of an immunoglobulin is to communicate to the immune system when the immunoglobulin binds its target. This is commonly referred to as “effector function.” Communication leads to antibody-dependent cellular cytotoxicity (ADCC), antibody-dependent cellular phagocytosis (ADCP), and/or complement dependent cytotoxicity (CDC). ADCC and ADCP are mediated through the binding of the Fc region to Fc receptors on the surface of cells of the immune system. CDC is mediated through the binding of the Fc with proteins of the complement system, e.g., Clq. In some embodiments, the multispecific antigen binding proteins of the invention comprise one or more amino acid substitutions in the constant region to enhance effector function, including ADCC activity, CDC activity, ADCP activity, and/or the clearance or half-life of the antigen binding protein. Exemplary amino acid substitutions (EU numbering) that can enhance effector function include, but are not limited to, E233L, L234I, L234Y, L235S, G236A, S239D, F243L, F243V, P247I, D280H, K290S, K290E, K290N, K290Y, R292P, E294L, Y296W, S298A, S298D, S298V, S298G, S298T, T299A, Y300L, V3051, Q311M, K326A, K326E, K326W, A330S, A330L, A330M, A330F, 1332E, D333A, E333S, E333A, K334A, K334V, A339D, A339Q, P396L, or combinations of any of the foregoing.

In other embodiments, the multispecific antigen binding proteins of the invention comprise one or more amino acid substitutions in the constant region to reduce effector function. Exemplary amino acid substitutions (EU numbering) that can reduce effector function include, but are not limited to, C220S, C226S, C229S, E233P, L234A, L234V, V234A, L234F, L235A, L235E, G237A, P238S, S267E, H268Q, N297A, N297G, V309L, E318A, L328F, A330S, A331S, P331S or combinations of any of the foregoing.

Glycosylation can contribute to the effector function of antibodies, particularly IgG1 antibodies. Thus, in some embodiments, the multispecific antigen binding proteins of the invention may comprise one or more amino acid substitutions that affect the level or type of glycosylation of the binding proteins. Glycosylation of polypeptides is typically either N-linked or O-linked. N-linked refers to the attachment of the carbohydrate moiety to the side chain of an asparagine residue. The tri-peptide sequences asparagine-X-serine and asparagine-X-threonine, where X is any amino acid except proline, are the recognition sequences for enzymatic attachment of the carbohydrate moiety to the asparagine side chain. Thus, the presence of either of these tri-peptide sequences in a polypeptide creates a potential glycosylation site. O-linked glycosylation refers to the attachment of one of the sugars N-acetylgalactosamine, galactose, or xylose, to a hydroxyamino acid, most commonly serine or threonine, although 5-hydroxyproline or 5-hydroxylysine may also be used.

In certain embodiments, glycosylation of the multispecific antigen binding proteins described herein is increased by adding one or more glycosylation sites, e.g., to the Fc region of the binding protein. Addition of glycosylation sites to the antigen binding protein can be conveniently accomplished by altering the amino acid sequence such that it contains one or more of the above-described tri-peptide sequences (for N-linked glycosylation sites). The alteration may also be made by the addition of, or substitution by, one or more serine or threonine residues to the starting sequence (for O-linked glycosylation sites). For ease, the antigen binding protein amino acid sequence may be altered through changes at the DNA level, particularly by mutating the DNA encoding the target polypeptide at preselected bases such that codons are generated that will translate into the desired amino acids.

The invention also encompasses production of multispecific antigen binding protein molecules with altered carbohydrate structure resulting in altered effector activity, including antigen binding proteins with absent or reduced fucosylation that exhibit improved ADCC activity. Various methods are known in the art to reduce or eliminate fucosylation. For example, ADCC effector activity is mediated by binding of the antibody molecule to the FcγRIII receptor, which has been shown to be dependent on the carbohydrate structure of the N-linked glycosylation at the N297 residue of the CH2 domain. Non-fucosylated antibodies bind this receptor with increased affinity and trigger FcγRIII-mediated effector functions more efficiently than native, fucosylated antibodies. For example, recombinant production of non-fucosylated antibody in CHO cells in which the alpha-1,6-fucosyl transferase enzyme has been knocked out results in antibody with 100-fold increased ADCC activity (see Yamane-Ohnuki et al., Biotechnol Bioeng. 87(5):614-22, 2004). Similar effects can be accomplished through decreasing the activity of alpha-1,6-fucosyl transferase enzyme or other enzymes in the fucosylation pathway, e.g., through siRNA or antisense RNA treatment, engineering cell lines to knockout the enzyme(s), or culturing with selective glycosylation inhibitors (see Rothman et al., Mol Immunol. 26(12):1113-23, 1989). Some host cell strains, e.g. Lec13 or rat hybridoma YB2/0 cell line naturally produce antibodies with lower fucosylation levels (see Shields et al., J Biol Chem. 277(30):26733-40, 2002 and Shinkawa et al., J Biol Chem. 278(5):3466-73, 2003). An increase in the level of bisected carbohydrate, e.g. through recombinantly producing antibody in cells that overexpress GnTIII enzyme, has also been determined to increase ADCC activity (see Umana et al., Nat Biotechnol. 17(2):176-80, 1999).

In other embodiments, glycosylation of the multispecific antigen binding proteins described herein is decreased or eliminated by removing one or more glycosylation sites, e.g., from the Fc region of the binding protein. Amino acid substitutions that eliminate or alter N-linked glycosylation sites can reduce or eliminate N-linked glycosylation of the antigen binding protein. In certain embodiments, the multispecific antigen binding proteins described herein comprise a mutation at position N297 (EU numbering), such as N297Q, N297A, or N297G. In one particular embodiment, the multispecific antigen binding proteins of the invention comprise a Fc region from a human IgG1 antibody with a N297G mutation. To improve the stability of molecules comprising a N297 mutation, the Fc region of the molecules may be further engineered. For instance, in some embodiments, one or more amino acids in the Fc region are substituted with cysteine to promote disulfide bond formation in the dimeric state. Residues corresponding to V259, A287, R292, V302, L306, V323, or 1332 (EU numbering) of an IgG1 Fc region may thus be substituted with cysteine. Preferably, specific pairs of residues are substituted with cysteine such that they preferentially form a disulfide bond with each other, thus limiting or preventing disulfide bond scrambling. Preferred pairs include, but are not limited to, A287C and L306C, V259C and L306C, R292C and V302C, and V323C and 1332C. In particular embodiments, the multispecific antigen binding proteins described herein comprise a Fc region from a human IgG1 antibody with mutations at R292C and V302C. In such embodiments, the Fc region may also comprise a N297G mutation.

Modifications of the multispecific antigen binding proteins of the invention to increase serum half-life also may desirable, for example, by incorporation of or addition of a salvage receptor binding epitope (e.g., by mutation of the appropriate region or by incorporating the epitope into a peptide tag that is then fused to the antigen binding protein at either end or in the middle, e.g., by DNA or peptide synthesis; see, e.g., WO96/32478) or adding molecules such as PEG or other water soluble polymers, including polysaccharide polymers. The salvage receptor binding epitope preferably constitutes a region wherein any one or more amino acid residues from one or two loops of a Fc region are transferred to an analogous position in the antigen binding protein. Even more preferably, three or more residues from one or two loops of the Fc region are transferred. Still more preferred, the epitope is taken from the CH2 domain of the Fc region (e.g., an IgG Fc region) and transferred to the CH1, CH3, or VH region, or more than one such region, of the antigen binding protein. Alternatively, the epitope is taken from the CH2 domain of the Fc region and transferred to the CL region or VL region, or both, of the antigen binding protein. See International applications WO 97/34631 and WO 96/32478 for a description of Fc variants and their interaction with the salvage receptor.

The present invention includes one or more isolated nucleic acids encoding the multispecific antigen binding proteins and components thereof described herein. Nucleic acid molecules of the invention include DNA and RNA in both single-stranded and double-stranded form, as well as the corresponding complementary sequences. DNA includes, for example, cDNA, genomic DNA, chemically synthesized DNA, DNA amplified by PCR, and combinations thereof. The nucleic acid molecules of the invention include full-length genes or cDNA molecules as well as a combination of fragments thereof. The nucleic acids of the invention are preferentially derived from human sources, but the invention includes those derived from non-human species, as well.

Relevant amino acid sequences from an immunoglobulin or region thereof (e.g. variable region, Fc region, etc.) or polypeptide of interest may be determined by direct protein sequencing, and suitable encoding nucleotide sequences can be designed according to a universal codon table.

Alternatively, genomic or cDNA encoding monoclonal antibodies from which the binding domains of the multispecific antigen binding proteins of the invention may be derived can be isolated and sequenced from cells producing such antibodies using conventional procedures (e.g., by using oligonucleotide probes that are capable of binding specifically to genes encoding the heavy and light chains of the monoclonal antibodies).

An “isolated nucleic acid,” which is used interchangeably herein with “isolated polynucleotide,” is a nucleic acid that has been separated from adjacent genetic sequences present in the genome of the organism from which the nucleic acid was isolated, in the case of nucleic acids isolated from naturally-occurring sources. In the case of nucleic acids synthesized enzymatically from a template or chemically, such as PCR products, cDNA molecules, or oligonucleotides for example, it is understood that the nucleic acids resulting from such processes are isolated nucleic acids. An isolated nucleic acid molecule refers to a nucleic acid molecule in the form of a separate fragment or as a component of a larger nucleic acid construct. In one preferred embodiment, the nucleic acids are substantially free from contaminating endogenous material. The nucleic acid molecule has preferably been derived from DNA or RNA isolated at least once in substantially pure form and in a quantity or concentration enabling identification, manipulation, and recovery of its component nucleotide sequences by standard biochemical methods (such as those outlined in Sambrook et al., Molecular Cloning: A Laboratory Manual, 2nd ed., Cold Spring Harbor Laboratory, Cold Spring Harbor, NY (1989)). Such sequences are preferably provided and/or constructed in the form of an open reading frame uninterrupted by internal non-translated sequences, or introns, that are typically present in eukaryotic genes. Sequences of non-translated DNA can be present 5′ or 3′ from an open reading frame, where the same do not interfere with manipulation or expression of the coding region. Unless specified otherwise, the left-hand end of any single-stranded polynucleotide sequence discussed herein is the 5′ end; the left-hand direction of double-stranded polynucleotide sequences is referred to as the 5′ direction. The direction of 5′ to 3′ production of nascent RNA transcripts is referred to as the transcription direction; sequence regions on the DNA strand having the same sequence as the RNA transcript that are 5′ to the 5′ end of the RNA transcript are referred to as “upstream sequences;” sequence regions on the DNA strand having the same sequence as the RNA transcript that are 3′ to the 3′ end of the RNA transcript are referred to as “downstream sequences.”

The present invention also includes nucleic acids that hybridize under moderately stringent conditions, and more preferably highly stringent conditions, to nucleic acids encoding polypeptides as described herein. The basic parameters affecting the choice of hybridization conditions and guidance for devising suitable conditions are set forth by Sambrook, Fritsch, and Maniatis (1989, Molecular Cloning: A Laboratory Manual, Cold Spring Harbor Laboratory Press, Cold Spring Harbor, N.Y., chapters 9 and 11; and Current Protocols in Molecular Biology, 1995, Ausubel et al., eds., John Wiley & Sons, Inc., sections 2.10 and 6.3-6.4), and can be readily determined by those having ordinary skill in the art based on, for example, the length and/or base composition of the DNA. One way of achieving moderately stringent conditions involves the use of a prewashing solution containing 5×SSC, 0.5% SDS, 1.0 mM EDTA (pH 8.0), hybridization buffer of about 50% formamide, 6×SSC, and a hybridization temperature of about 55° C. (or other similar hybridization solutions, such as one containing about 50% formamide, with a hybridization temperature of about 42° C.), and washing conditions of about 60° C., in 0.5×SSC, 0.1% SDS. Generally, highly stringent conditions are defined as hybridization conditions as above, but with washing at approximately 68° C., 0.2×SSC, 0.1% SDS. SSPE (1×SSPE is 0.15M NaCl, 10 mM NaH₂PO₄, and 1.25 mM EDTA, pH 7.4) can be substituted for SSC (1×SSC is 0.15M NaCl and 15 mM sodium citrate) in the hybridization and wash buffers; washes are performed for 15 minutes after hybridization is complete. It should be understood that the wash temperature and wash salt concentration can be adjusted as necessary to achieve a desired degree of stringency by applying the basic principles that govern hybridization reactions and duplex stability, as known to those skilled in the art and described further below (see, e.g., Sambrook et al., 1989). When hybridizing a nucleic acid to a target nucleic acid of unknown sequence, the hybrid length is assumed to be that of the hybridizing nucleic acid. When nucleic acids of known sequence are hybridized, the hybrid length can be determined by aligning the sequences of the nucleic acids and identifying the region or regions of optimal sequence complementarity. The hybridization temperature for hybrids anticipated to be less than 50 base pairs in length should be 5 to 10° C. less than the melting temperature (Tm) of the hybrid, where Tm is determined according to the following equations. For hybrids less than 18 base pairs in length, Tm (° C.)=2(#of A+T bases)+4(#of G+C bases). For hybrids above 18 base pairs in length, Tm (° C.)=81.5+16.6(log 10 [Na+])+0.41(% G+C)−(600/N), where N is the number of bases in the hybrid, and [Na+] is the concentration of sodium ions in the hybridization buffer ([Na+] for 1×SSC=0.165M). Preferably, each such hybridizing nucleic acid has a length that is at least 15 nucleotides (or more preferably at least 18 nucleotides, or at least 20 nucleotides, or at least 25 nucleotides, or at least 30 nucleotides, or at least 40 nucleotides, or most preferably at least 50 nucleotides), or at least 25% (more preferably at least 50%, or at least 60%, or at least 70%, and most preferably at least 80%) of the length of the nucleic acid of the present invention to which it hybridizes, and has at least 60% sequence identity (more preferably at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, and most preferably at least 99.5%) with the nucleic acid of the present invention to which it hybridizes, where sequence identity is determined by comparing the sequences of the hybridizing nucleic acids when aligned so as to maximize overlap and identity while minimizing sequence gaps as described in more detail above.

Variants of the antigen binding proteins described herein can be prepared by site-specific mutagenesis of nucleotides in the DNA encoding the polypeptide, using cassette or PCR mutagenesis or other techniques well known in the art, to produce DNA encoding the variant, and thereafter expressing the recombinant DNA in cell culture as outlined herein. However, antigen binding proteins comprising variant CDRs having up to about 100-150 residues may be prepared by in vitro synthesis using established techniques. The variants typically exhibit the same qualitative biological activity as the naturally occurring analogue, e.g., binding to antigen. Such variants include, for example, deletions and/or insertions and/or substitutions of residues within the amino acid sequences of the antigen binding proteins. Any combination of deletion, insertion, and substitution is made to arrive at the final construct, provided that the final construct possesses the desired characteristics. The amino acid changes also may alter post-translational processes of the antigen binding protein, such as changing the number or position of glycosylation sites. In certain embodiments, antigen binding protein variants are prepared with the intent to modify those amino acid residues which are directly involved in epitope binding. In other embodiments, modification of residues which are not directly involved in epitope binding or residues not involved in epitope binding in any way, is desirable, for purposes discussed herein. Mutagenesis within any of the CDR regions and/or framework regions is contemplated. Covariance analysis techniques can be employed by the skilled artisan to design useful modifications in the amino acid sequence of the antigen binding protein. See, e.g., Choulier, et al., Proteins 41:475-484, 2000; Demarest et al., J. Mol. Biol. 335:41-48, 2004; Hugo et al., Protein Engineering 16(5):381-86, 2003; Aurora et al., US Patent Publication No. 2008/0318207 A1; Glaser et al., US Patent Publication No. 2009/0048122 A1; Urech et al., WO 2008/110348 A1; Borras et al., WO 2009/000099 A2. Such modifications determined by covariance analysis can improve potency, pharmacokinetic, pharmacodynamic, and/or manufacturability characteristics of an antigen binding protein.

The present invention also includes vectors comprising one or more nucleic acids encoding one or more components of the multispecific antigen binding proteins of the invention (e.g. variable regions, light chains, heavy chains, modified heavy chains, and Fd fragments). The term “vector” refers to any molecule or entity (e.g., nucleic acid, plasmid, bacteriophage or virus) used to transfer protein coding information into a host cell. Examples of vectors include, but are not limited to, plasmids, viral vectors, non-episomal mammalian vectors and expression vectors, for example, recombinant expression vectors. The term “expression vector” or “expression construct” as used herein refers to a recombinant DNA molecule containing a desired coding sequence and appropriate nucleic acid control sequences necessary for the expression of the operably linked coding sequence in a particular host cell. An expression vector can include, but is not limited to, sequences that affect or control transcription, translation, and, if introns are present, affect RNA splicing of a coding region operably linked thereto. Nucleic acid sequences necessary for expression in prokaryotes include a promoter, optionally an operator sequence, a ribosome binding site and possibly other sequences. Eukaryotic cells are known to utilize promoters, enhancers, and termination and polyadenylation signals. A secretory signal peptide sequence can also, optionally, be encoded by the expression vector, operably linked to the coding sequence of interest, so that the expressed polypeptide can be secreted by the recombinant host cell, for more facile isolation of the polypeptide of interest from the cell, if desired. In certain embodiments, a signal peptide is selected from the group consisting of MDMRVPAQLLGLLLLWLRGARC (SEQ ID NO: 52), MAWALLLLTLLTQGTGSWA (SEQ ID NO: 53), MTCSPLLLTLLIHCTGSWA (SEQ ID NO: 54), MEAPAQLLFLLLLWLPDTTG (SEQ ID NO: 55), MEWTWRVLFLVAAATGAHS (SEQ ID NO: 56), METPAQLLFLLLLWLPDTTG (SEQ ID NO: 57), METPAQLLFLLLLWLPDTTG (SEQ ID NO: 58), MKHLWFFLLLVAAPRWVLS (SEQ ID NO: 59), and MEWSWVFLFFLSVTTGVHS (SEQ ID NO: 60).

Typically, expression vectors used in the host cells to produce the multispecific antigen proteins of the invention will contain sequences for plasmid maintenance and for cloning and expression of exogenous nucleotide sequences encoding the components of the multispecific antigen binding proteins. Such sequences, collectively referred to as “flanking sequences,” in certain embodiments will typically include one or more of the following nucleotide sequences: a promoter, one or more enhancer sequences, an origin of replication, a transcriptional termination sequence, a complete intron sequence containing a donor and acceptor splice site, a sequence encoding a leader sequence for polypeptide secretion, a ribosome binding site, a polyadenylation sequence, a polylinker region for inserting the nucleic acid encoding the polypeptide to be expressed, and a selectable marker element. Each of these sequences is discussed below.

Optionally, the vector may contain a “tag”-encoding sequence, i.e., an oligonucleotide molecule located at the 5′ or 3′ end of the polypeptide coding sequence; the oligonucleotide tag sequence encodes polyHis (such as hexaHis), streptavidin, FLAG, HA (hemaglutinin influenza virus), myc, or another “tag” molecule for which commercially available antibodies exist. This tag is typically fused to the polypeptide upon expression of the polypeptide, and can serve as a means for affinity purification or detection of the polypeptide from the host cell. Affinity purification can be accomplished, for example, by column chromatography using antibodies against the tag as an affinity matrix. Optionally, the tag can subsequently be removed from the purified polypeptide by various means such as using certain peptidases for cleavage.

Flanking sequences may be homologous (i.e., from the same species and/or strain as the host cell), heterologous (i.e., from a species other than the host cell species or strain), hybrid (i.e., a combination of flanking sequences from more than one source), synthetic or native. As such, the source of a flanking sequence may be any prokaryotic or eukaryotic organism, any vertebrate or invertebrate organism, or any plant, provided that the flanking sequence is functional in, and can be activated by, the host cell machinery.

Flanking sequences useful in the vectors of this invention may be obtained by any of several methods well known in the art. Typically, flanking sequences useful herein will have been previously identified by mapping and/or by restriction endonuclease digestion and can thus be isolated from the proper tissue source using the appropriate restriction endonucleases. In some cases, the full nucleotide sequence of a flanking sequence may be known. Here, the flanking sequence may be synthesized using routine methods for nucleic acid synthesis or cloning.

Whether all or only a portion of the flanking sequence is known, it may be obtained using polymerase chain reaction (PCR) and/or by screening a genomic library with a suitable probe such as an oligonucleotide and/or flanking sequence fragment from the same or another species. Where the flanking sequence is not known, a fragment of DNA containing a flanking sequence may be isolated from a larger piece of DNA that may contain, for example, a coding sequence or even another gene or genes. Isolation may be accomplished by restriction endonuclease digestion to produce the proper DNA fragment followed by isolation using agarose gel purification, Qiagen® column chromatography (Chatsworth, CA), or other methods known to the skilled artisan. The selection of suitable enzymes to accomplish this purpose will be readily apparent to one of ordinary skill in the art.

An origin of replication is typically a part of those prokaryotic expression vectors purchased commercially, and the origin aids in the amplification of the vector in a host cell. If the vector of choice does not contain an origin of replication site, one may be chemically synthesized based on a known sequence, and ligated into the vector. For example, the origin of replication from the plasmid pBR322 (New England Biolabs, Beverly, MA) is suitable for most gram-negative bacteria, and various viral origins (e.g., SV40, polyoma, adenovirus, vesicular stomatitus virus (VSV), or papillomaviruses such as HPV or BPV) are useful for cloning vectors in mammalian cells. Generally, the origin of replication component is not needed for mammalian expression vectors (for example, the SV40 origin is often used only because it also contains the virus early promoter).

A transcription termination sequence is typically located 3′ to the end of a polypeptide coding region and serves to terminate transcription. Usually, a transcription termination sequence in prokaryotic cells is a G-C rich fragment followed by a poly-T sequence. While the sequence is easily cloned from a library or even purchased commercially as part of a vector, it can also be readily synthesized using known methods for nucleic acid synthesis.

A selectable marker gene encodes a protein necessary for the survival and growth of a host cell grown in a selective culture medium. Typical selection marker genes encode proteins that (a) confer resistance to antibiotics or other toxins, e.g., ampicillin, tetracycline, or kanamycin for prokaryotic host cells; (b) complement auxotrophic deficiencies of the cell; or (c) supply critical nutrients not available from complex or defined media. Specific selectable markers are the kanamycin resistance gene, the ampicillin resistance gene, and the tetracycline resistance gene. Advantageously, a neomycin resistance gene may also be used for selection in both prokaryotic and eukaryotic host cells.

Other selectable genes may be used to amplify the gene that will be expressed. Amplification is the process wherein genes that are required for production of a protein critical for growth or cell survival are reiterated in tandem within the chromosomes of successive generations of recombinant cells. Examples of suitable selectable markers for mammalian cells include dihydrofolate reductase (DHFR) and promoterless thymidine kinase genes. Mammalian cell transformants are placed under selection pressure wherein only the transformants are uniquely adapted to survive by virtue of the selectable gene present in the vector. Selection pressure is imposed by culturing the transformed cells under conditions in which the concentration of selection agent in the medium is successively increased, thereby leading to the amplification of both the selectable gene and the DNA that encodes another gene, such as one or more components of the multispecific antigen binding proteins described herein. As a result, increased quantities of a polypeptide are synthesized from the amplified DNA.

A ribosome-binding site is usually necessary for translation initiation of mRNA and is characterized by a Shine-Dalgarno sequence (prokaryotes) or a Kozak sequence (eukaryotes). The element is typically located 3′ to the promoter and 5′ to the coding sequence of the polypeptide to be expressed. In certain embodiments, one or more coding regions may be operably linked to an internal ribosome binding site (IRES), allowing translation of two open reading frames from a single RNA transcript.

In some cases, such as where glycosylation is desired in a eukaryotic host cell expression system, one may manipulate the various pre- or prosequences to improve glycosylation or yield. For example, one may alter the peptidase cleavage site of a particular signal peptide, or add prosequences, which also may affect glycosylation. The final protein product may have, in the −1 position (relative to the first amino acid of the mature protein) one or more additional amino acids incident to expression, which may not have been totally removed. For example, the final protein product may have one or two amino acid residues found in the peptidase cleavage site, attached to the amino-terminus. Alternatively, use of some enzyme cleavage sites may result in a slightly truncated form of the desired polypeptide, if the enzyme cuts at such area within the mature polypeptide.

Expression and cloning vectors of the invention will typically contain a promoter that is recognized by the host organism and operably linked to the molecule encoding the polypeptide. The term “operably linked” as used herein refers to the linkage of two or more nucleic acid sequences in such a manner that a nucleic acid molecule capable of directing the transcription of a given gene and/or the synthesis of a desired protein molecule is produced. For example, a control sequence in a vector that is “operably linked” to a protein coding sequence is ligated thereto so that expression of the protein coding sequence is achieved under conditions compatible with the transcriptional activity of the control sequences. More specifically, a promoter and/or enhancer sequence, including any combination of cis-acting transcriptional control elements is operably linked to a coding sequence if it stimulates or modulates the transcription of the coding sequence in an appropriate host cell or other expression system.

Promoters are untranscribed sequences located upstream (i.e., 5′) to the start codon of a structural gene (generally within about 100 to 1000 bp) that control transcription of the structural gene. Promoters are conventionally grouped into one of two classes: inducible promoters and constitutive promoters. Inducible promoters initiate increased levels of transcription from DNA under their control in response to some change in culture conditions, such as the presence or absence of a nutrient or a change in temperature. Constitutive promoters, on the other hand, uniformly transcribe a gene to which they are operably linked, that is, with little or no control over gene expression. A large number of promoters, recognized by a variety of potential host cells, are well known. A suitable promoter is operably linked to the DNA encoding e.g., heavy chain, light chain, modified heavy chain, or other component of the multispecific antigen binding proteins of the invention, by removing the promoter from the source DNA by restriction enzyme digestion and inserting the desired promoter sequence into the vector.

Suitable promoters for use with yeast hosts are also well known in the art. Yeast enhancers are advantageously used with yeast promoters. Suitable promoters for use with mammalian host cells are well known and include, but are not limited to, those obtained from the genomes of viruses such as polyoma virus, fowlpox virus, adenovirus (such as Adenovirus 2), bovine papilloma virus, avian sarcoma virus, cytomegalovirus, retroviruses, hepatitis-B virus and most preferably Simian Virus 40 (SV40). Other suitable mammalian promoters include heterologous mammalian promoters, for example, heat-shock promoters and the actin promoter.

Additional promoters which may be of interest include, but are not limited to: SV40 early promoter (Benoist and Chambon, 1981, Nature 290:304-310); CMV promoter (Thomsen et al., 1984, Proc. Natl. Acad. U.S.A. 81:659-663); the promoter contained in the 3′ long terminal repeat of Rous sarcoma virus (Yamamoto et al., 1980, Cell 22:787-797); herpes thymidine kinase promoter (Wagner et al., 1981, Proc. Natl. Acad. Sci. U.S.A. 78: 1444-1445); promoter and regulatory sequences from the metallothionine gene Prinster et al., 1982, Nature 296:39-42); and prokaryotic promoters such as the beta-lactamase promoter (Villa-Kamaroff et al., 1978, Proc. Natl. Acad. Sci. U.S.A. 75:3727-3731); or the tac promoter (DeBoer et al., 1983, Proc. Natl. Acad. Sci. U.S.A. 80:21-25). Also of interest are the following animal transcriptional control regions, which exhibit tissue specificity and have been utilized in transgenic animals: the elastase I gene control region that is active in pancreatic acinar cells (Swift et al., 1984, Cell 38:639-646; Ornitz et al., 1986, Cold Spring Harbor Symp. Quant. Biol. 50:399-409; MacDonald, 1987, Hepatology 7:425-515); the insulin gene control region that is active in pancreatic beta cells (Hanahan, 1985, Nature 315: 115-122); the immunoglobulin gene control region that is active in lymphoid cells (Grosschedl et al., 1984, Cell 38:647-658; Adames et al., 1985, Nature 318:533-538; Alexander et al., 1987, Mol. Cell. Biol. 7: 1436-1444); the mouse mammary tumor virus control region that is active in testicular, breast, lymphoid and mast cells (Leder et al., 1986, Cell 45:485-495); the albumin gene control region that is active in liver (Pinkert et al., 1987, Genes and Devel. 1:268-276); the alpha-feto-protein gene control region that is active in liver (Krumlauf et al., 1985, Mol. Cell. Biol. 5: 1639-1648; Hammer et al., 1987, Science 253:53-58); the alpha 1-antitrypsin gene control region that is active in liver (Kelsey et al., 1987, Genes and Devel. 1: 161-171); the beta-globin gene control region that is active in myeloid cells (Mogram et al, 1985, Nature 315:338-340; Kollias et al, 1986, Cell 46:89-94); the myelin basic protein gene control region that is active in oligodendrocyte cells in the brain (Readhead et al., 1987, Cell 48:703-712); the myosin light chain-2 gene control region that is active in skeletal muscle (Sani, 1985, Nature 314:283-286); and the gonadotropic releasing hormone gene control region that is active in the hypothalamus (Mason et al., 1986, Science 234: 1372-1378).

An enhancer sequence may be inserted into the vector to increase transcription of DNA encoding a component of the multispecific antigen binding proteins (e.g., light chain, heavy chain, modified heavy chain, Fd fragment) by higher eukaryotes. Enhancers are cis-acting elements of DNA, usually about 10-300 bp in length, that act on the promoter to increase transcription. Enhancers are relatively orientation and position independent, having been found at positions both 5′ and 3′ to the transcription unit. Several enhancer sequences available from mammalian genes are known (e.g., globin, elastase, albumin, alpha-feto-protein and insulin). Typically, however, an enhancer from a virus is used. The SV40 enhancer, the cytomegalovirus early promoter enhancer, the polyoma enhancer, and adenovirus enhancers known in the art are exemplary enhancing elements for the activation of eukaryotic promoters. While an enhancer may be positioned in the vector either 5′ or 3′ to a coding sequence, it is typically located at a site 5′ from the promoter. A sequence encoding an appropriate native or heterologous signal sequence (leader sequence or signal peptide) can be incorporated into an expression vector, to promote extracellular secretion of the antibody. The choice of signal peptide or leader depends on the type of host cells in which the antibody is to be produced, and a heterologous signal sequence can replace the native signal sequence. Examples of signal peptides are described above. Other signal peptides that are functional in mammalian host cells include the signal sequence for interleukin-7 (IL-7) described in U.S. Pat. No. 4,965,195; the signal sequence for interleukin-2 receptor described in Cosman et al.,1984, Nature 312:768; the interleukin-4 receptor signal peptide described in EP Patent No. 0367 566; the type I interleukin-1 receptor signal peptide described in U.S. Pat. No. 4,968,607; the type II interleukin-1 receptor signal peptide described in EP Patent No. 0 460 846.

The expression vectors that are provided may be constructed from a starting vector such as a commercially available vector. Such vectors may or may not contain all of the desired flanking sequences. Where one or more of the flanking sequences described herein are not already present in the vector, they may be individually obtained and ligated into the vector. Methods used for obtaining each of the flanking sequences are well known to one skilled in the art. The expression vectors can be introduced into host cells to thereby produce proteins, including fusion proteins, encoded by nucleic acids as described herein.

In certain embodiments, nucleic acids encoding the different components of the multispecific antigen binding proteins of the invention may be inserted into the same expression vector. In such embodiments, the two nucleic acids may be separated by an internal ribosome entry site (IRES) and under the control of a single promoter such that the light chain and heavy chain are expressed from the same mRNA transcript. Alternatively, the two nucleic acids may be under the control of two separate promoters such that the light chain and heavy chain are expressed from two separate mRNA transcripts.

Similarly, for IgG-scFv multispecific antigen binding proteins, the nucleic acid encoding the light chain may be cloned into the same expression vector as the nucleic acid encoding the modified heavy chain (fusion protein comprising the heavy chain and scFv) where the two nucleic acids are under the control of a single promoter and separated by an IRES or where the two nucleic acids are under the control of two separate promoters. For IgG-Fab multispecific antigen binding proteins, nucleic acids encoding each of the three components may be cloned into the same expression vector. In some embodiments, the nucleic acid encoding the light chain of the IgG-Fab molecule and the nucleic acid encoding the second polypeptide (which comprises the other half of the C-terminal Fab domain) are cloned into one expression vector, whereas the nucleic acid encoding the modified heavy chain (fusion protein comprising a heavy chain and half of a Fab domain) is cloned into a second expression vector. In certain embodiments, all components of the multispecific antigen binding proteins described herein are expressed from the same host cell population. For example, even if one or more components is cloned into a separate expression vector, the host cell is co-transfected with both expression vectors such that one cell produces all components of the multispecific antigen binding proteins.

After the vector has been constructed and the one or more nucleic acid molecules encoding the components of the multispecific antigen binding proteins described herein has been inserted into the proper site(s) of the vector or vectors, the completed vector(s) may be inserted into a suitable host cell for amplification and/or polypeptide expression. Thus, the present invention encompasses an isolated host cell comprising one or more expression vectors encoding the components of the multispecific antigen binding proteins. The term “host cell” as used herein refers to a cell that has been transformed, or is capable of being transformed, with a nucleic acid and thereby expresses a gene of interest. The term includes the progeny of the parent cell, whether or not the progeny is identical in morphology or in genetic make-up to the original parent cell, so long as the gene of interest is present. A host cell that comprises an isolated nucleic acid of the invention, preferably operably linked to at least one expression control sequence (e.g. promoter or enhancer), is a “recombinant host cell.”

The transformation of an expression vector for an antigen binding protein into a selected host cell may be accomplished by well-known methods including transfection, infection, calcium phosphate co-precipitation, electroporation, microinjection, lipofection, DEAE-dextran mediated transfection, or other known techniques. The method selected will in part be a function of the type of host cell to be used. These methods and other suitable methods are well known to the skilled artisan, and are set forth, for example, in Sambrook et al., 2001, supra.

A host cell, when cultured under appropriate conditions, synthesizes an antigen binding protein that can subsequently be collected from the culture medium (if the host cell secretes it into the medium) or directly from the host cell producing it (if it is not secreted). The selection of an appropriate host cell will depend upon various factors, such as desired expression levels, polypeptide modifications that are desirable or necessary for activity (such as glycosylation or phosphorylation) and ease of folding into a biologically active molecule.

Exemplary host cells include prokaryote, yeast, or higher eukaryote cells. Prokaryotic host cells include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacillus, such as B. subtilis and B. licheniformis, Pseudomonas, and Streptomyces. Eukaryotic microbes such as filamentous fungi or yeast are suitable cloning or expression hosts for recombinant polypeptides. Saccharomyces cerevisiae, or common baker's yeast, is the most commonly used among lower eukaryotic host microorganisms. However, a number of other genera, species, and strains are commonly available and useful herein, such as Pichia, e.g. P. pastoris, Schizosaccharomyces pombe; Kluyveromyces, Yarrowia; Candida; Trichoderma reesia; Neurospora crassa; Schwanniomyces, such as Schwanniomyces occidentalis; and filamentous fungi, such as, e.g., Neurospora, Penicillium, Tolypocladium, and Aspergillus hosts such as A. nidulans and A. niger.

Host cells for the expression of glycosylated antigen binding proteins can be derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection of such cells are publicly available, e.g., the L-1 variant of Autographa californica NPV and the Bm-5 strain of Bombyx mori NPV.

Vertebrate host cells are also suitable hosts, and recombinant production of antigen binding proteins from such cells has become routine procedure. Mammalian cell lines available as hosts for expression are well known in the art and include, but are not limited to, immortalized cell lines available from the American Type Culture Collection (ATCC), including but not limited to Chinese hamster ovary (CHO) cells, including CHOK1 cells (ATCC CCL61), DXB-11, DG-44, and Chinese hamster ovary cells/-DHFR (CHO, Urlaub et al., Proc. Natl. Acad. Sci. USA 77: 4216, 1980); monkey kidney CV1 line transformed by SV40 (COS-7, ATCC CRL 1651); human embryonic kidney line (293 or 293 cells subcloned for growth in suspension culture, (Graham et al., J. Gen Virol. 36: 59, 1977); baby hamster kidney cells (BHK, ATCC CCL 10); mouse sertoli cells (TM4, Mather, Biol. Reprod. 23: 243-251, 1980); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human hepatoma cells (Hep G2, HB 8065); mouse mammary tumor (MMT 060562, ATCC CCL51); TRI cells (Mather et al., Annals N.Y Acad. Sci. 383: 44-68, 1982); MRC 5 cells or FS4 cells; mammalian myeloma cells, and a number of other cell lines. In another embodiment, a cell line from the B cell lineage that does not make its own antibody but has a capacity to make and secrete a heterologous antibody can be selected. CHO cells are preferred host cells in some embodiments for expressing the multispecific antigen binding proteins of the invention.

Host cells are transformed or transfected with the above-described nucleic acids or vectors for production of multispecific antigen binding proteins and are cultured in conventional nutrient media modified as appropriate for inducing promoters, selecting transformants, or amplifying the genes encoding the desired sequences. In addition, novel vectors and transfected cell lines with multiple copies of transcription units separated by a selective marker are particularly useful for the expression of antigen binding proteins. Thus, the present invention also provides a method for preparing a multispecific antigen binding protein described herein comprising culturing a host cell comprising one or more expression vectors described herein in a culture medium under conditions permitting expression of the multispecific antigen binding protein encoded by the one or more expression vectors; and recovering the multispecific antigen binding protein from the culture medium.

The host cells used to produce the antigen binding proteins of the invention may be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. In addition, any of the media described in Ham et al., Meth. Enz. 58: 44, 1979; Barnes et al., Anal. Biochem. 102: 255, 1980; U.S. Pat. Nos. 4,767,704; 4,657,866; 4,927,762; 4,560,655; or 5,122,469; WO90103430; WO 87/00195; or U.S. Patent Re. No. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as Gentamycin™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

Upon culturing the host cells, the multispecific antigen binding protein can be produced intracellularly, in the periplasmic space, or directly secreted into the medium. If the antigen binding protein is produced intracellularly, as a first step, the particulate debris, either host cells or lysed fragments, is removed, for example, by centrifugation or ultrafiltration. The bispecifc antigen binding protein can be purified using, for example, hydroxyapatite chromatography, cation or anion exchange chromatography, or preferably affinity chromatography, using the antigen(s) of interest or protein A or protein G as an affinity ligand. Protein A can be used to purify proteins that include polypeptides that are based on human γ1, γ2, or γ4 heavy chains (Lindmark et al., J. Immunol. Meth. 62: 1-13, 1983). Protein G is recommended for all mouse isotypes and for human γ3 (Guss et al., EMBO J. 5: 15671575, 1986). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the protein comprises a CH3 domain, the Bakerbond ABX□□resin (J. T. Baker, Phillipsburg, N.J.) is useful for purification. Other techniques for protein purification such as ethanol precipitation, Reverse Phase HPLC, chromatofocusing, SDS-PAGE, and ammonium sulfate precipitation are also possible depending on the particular multispecific antigen binding protein to be recovered.

### EXAMPLES

### Materials and Methods

### Plasmid Construction

The antibody HC and LC genes were synthesized by Twist Bioscience and then individually cloned into a mammalian transient expression vector using a Golden Gate assembly method³⁰. To reduce protein heterogenicity, all HCs were constructed with human IgG1 scaffold (IgG1-SEFL2) carrying an aglycosylation mutation and a novel engineered disulfide bond³¹. For Hetero-Fcs that require HC heterodimerization, charge pair mutations (CPMs) were introduced into the Fc regions. After sequencing confirmation by Sanger, transfection-grade DNA were prepared using Qiagen Maxi plasmid purification kit, and then mixed at a ratio of 1:1 (HC:LC) for monoclonal antibodies and hybrid IgGs (non-cognate HC/LC pairs), 1:1:1:1 (HC1:LC1:HC2:LC2) for 4-chain Hetero-Fcs and 2:1:1 (cLC:HC1:HC2) for cLC Hetero-Fcs (3-chain Hetero-Fcs).

### Cell Culture and Protein Expression

All proteins were transiently expressed in suspension human embryonic kidney HEK293-6E cells (NRC-BRI) using an in-house improved protocol (Ref Grace's paper). Briefly, cells were maintained in FreeStyle F-17 medium (Thermo Fisher) with 0.1% Kolliphor P188 (Sigma), 25 μg/ml G418 (Gibco) and 6 mM L-glutamine (Invitrogen). To achieve a density of 2×10⁶ viable cells per ml for optimal transfection, cells were passaged 26 hours in advance of transfection. For each ml of cells, 0.5 μg DNA was complexed with 1.5 μl PEImax reagent (Polysciences) in 100 μl FreeStyle F-17 medium for 10 min, and then added to cell culture. One day after transfection, cell cultures were fed with Tryptone N1 solution (Organotechnie) and glucose (Thermo Fisher) to a final concentration of 2.5 g/L and 4.5 g/L, respectively. Three days later, 3.75 mM sodium valproate (MP Biomedicals) was added to enhance protein expression. At day 6 post transfection, conditioned medium was harvested for purification.

High-Throughput Protein Purification with ProA Magnetic Beads

The KingFisher® Flex system (Thermo Fisher) was used for high-throughput protein purification with magnetic ProA beads (GE Life Sciences). Briefly, 4 ml HEK293-6E cells in 24-well deep blocks were transfected for protein expression, followed by addition of 100 μl magnetic ProA beads were added one day before harvest. Then, beads were collected and subjected to KingFisher purification with a 24 deep-well magnetic head. After washing 3 times with PBS and twice with Milli-Q water, proteins were eluted with 500 μl of 100 mM sodium acetate at pH 3.6 for 10 min, and then immediately neutralized by adding 10 μl of 3 M Tris, pH 11.0.

Two-Step Purification with ProA and Cation Ion Exchange Chromatography

Proteins expressed in 40 ml HEK293-6E cells were purified using ProA affinity capture (1 ml HiTrap MabSelect SuRe, GE Life Sciences), eluted with 100 mM sodium acetate, pH 3.6 followed immediately by buffer exchange into 10 mM sodium acetate, 150 mM NaCl, pH 5.2 using a 5 ml HiTrap Desalting column (GE Life Sciences) as described previously³².

For ion-exchange chromatography, ProA eluates (1.5-1.8 ml) were diluted with 20 ml of 20 mM MES, pH 6.2 and loaded onto 1 ml cation ion-exchange column (SP-HP HiTrap, GE Life Sciences) at 1 ml/min. The column was washed with 8 column volumes of the same buffer at 1 ml/min and the proteins were eluted with a linear 0-400 mM NaCl gradient over 40 column volumes at 0.4 ml/min. Fractions of 90% or higher purity (as determined by SEC and MS) were pooled and their concentration was determined using Multiscan GO microplate reader (Thermo Fisher) 33.

### Non-Reducing SDS-PAGE

To analyze ProA purified samples, 1-2 μg of proteins were loaded onto Novex 4-20% Tris-Glycine gels (Invitrogen) in the absence of reducing agent. A PageRuler prestained protein ladder 10-250kD (Thermo Fisher) was included on each gel. After running at 150 V for 1 hour, the gel was stained using InstantBlue Coomassie Protein Stain (Expedeon), briefly washed with Milli-Q water, and then imaged with a Gel Doc System (Bio-Rad).

### Protein Mass Spectrometry by Liquid Chromatography-Mass Spectrometry (LC-MS)

Quantitation of protein species was performed on a high-resolution LC-MS system as previously described 34 with some modification. Briefly, about 15 μg of each purified sample was analyzed by non-reduced LC-MS to maintain sample integrity and retain chain pairing information. The LC-MS system consisted of an Agilent 1290 Infinity II UPLC connected to an Agilent 6224 ESI-TOF mass spectrometer. Chromatographic separation utilized a Zorbax RRHD 300SB-C8 2.1 X 50 mm, 1.8 um UPLC column heated to 70° C. and run at a flow rate of 0.5 ml/min. The MS method scans m/z [1000-7000] acquiring 0.7 spectra/sec. The resulting spectra were summed, then deconvoluted using either the Agilent Mass Hunter Qualitative Analysis software (Version B.07.00) or Intact Program module from Protein Metrics. An Excel-based tool was used to calculate the intact mass and intensity of various correct and mispaired species. In case of a 4-chain Hetero-Fc, the following species are calculated: IgG species with four unique chains, IgG species with HC heterodimer but 2× of one LC, IgG species with HC homodimer, and ½ Ab species.

### Characterization of Binding Affinity

To measure the binding affinities (K_{D} equilibrium dissociation constant) of the purified antibodies (bispecific or monoclonal) to soluble antigens, the antibodies were first captured onto streptavidin SAX biosensor tips with a biotinylated, polyclonal capture antibody (Jackson ImmunoResearch) and then incubated with a dilution series of each soluble antigen. This assay format was chosen so the bivalent antibodies would be immobilized and on the biosensor tips and tested versus the same serial dilution of each soluble antigen. The measured quantitative K_{D} affinities thus represent monovalent 1:1 binding interaction and can be directly compared. Experiments were run in a ForteBio Octet HTX instrument using the 96-tip mode with standard 5 Hz data acquisition rate at 27° C. and 1000 RPM. Using Genedata Screener V16 software, raw Octet binding data was processed with installed SPR kinetic curve fitting package and globally fit to a 1:1 binding model to determine the association rate constant (kₐ) and the dissociation rate constant (k_{d}). The equilibrium dissociation constant (K_{D}) was then calculated the as a ratio of k_{d}/kₐ.

### Results

### Diversity of Antibody Variable Regions Provides Rationale for the Chain Selectivity Assessment

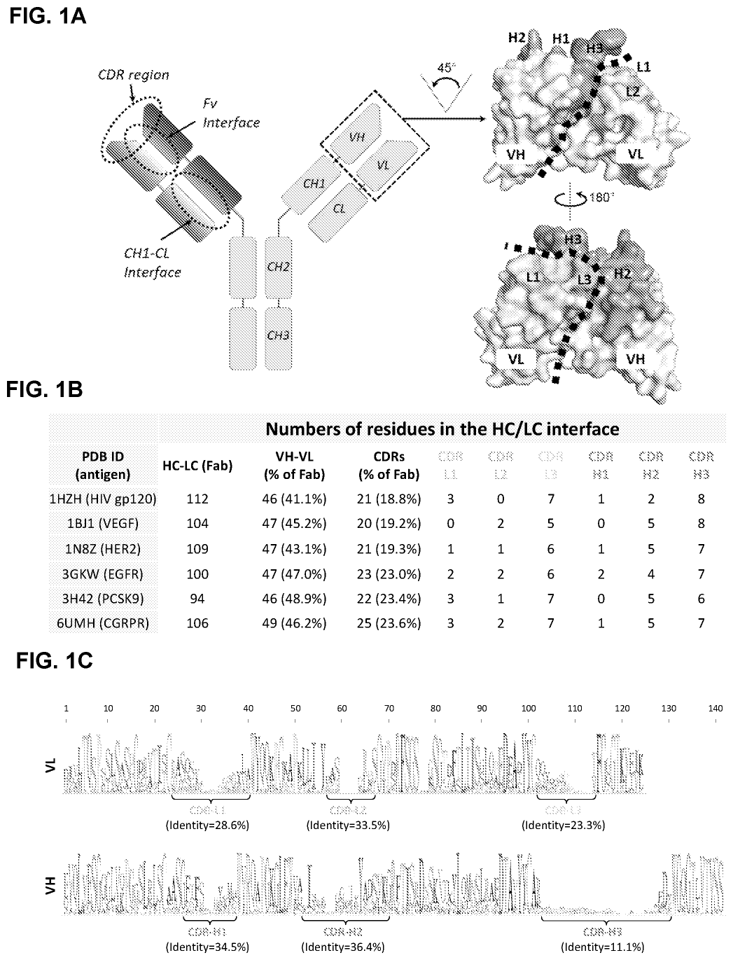

The IgG molecule is composed of two fragment antigen binding (Fab) regions that recognize the epitope and a fragment crystallizable (Fc) region that interacts with receptors on the cell surface (FIG. 1A). In the Fab region, two main points of contact mediate the HC/LC interaction: the VH/VL and CH1/CL domains. Although antibodies mostly conserve the CH1/CL interface (with only the LC kappa/lambda diversity to account for), the VH-VL interface is highly variable and unique to each antibody. Indeed, here both framework (FW) and complementary determining regions (CDRs) directly engage across the interface (FIG. 1A). To further evaluate the native properties of the VH/VL interface, 6 x-ray structures of Abs generated against a variety of relevant therapeutic targets were analyzed. Across these 6 structures, it was calculated that ˜100 (94 to 112) residues mediate the HC/LC interface (buried surface area of 1,620-1,880 Å²) with over 40% of those locate in the VH/VL interface, with the CDRs contributing ˜20% of the total (FIG. 1B). Among all 6 CDRs, the CDR-H2 and CDR-H3 in the HC and CDR-L3 in the LC have the most interfacing residues and are responsible for over 15% of the overall HC/LC interaction. Since CDR-H3 and CDR-L3 are major determinants for antibody target specificity, they are highly variable displaying low sequence identity (11.1 and 23.3%, respectively (FIG. 1C)). Due to the fact that highly diverse CDRs can contribute to HC/LC interaction, it was hypothesized that some HC/LC pairs and interfaces may be more favorable than others. Moreover, as the VH/VL interaction is reportedly the first step during the assembly of the quaternary IgG structure^{21,22}, a favorable VH/VL pair may be key to prime the cognate HC/LC pairing. Therefore, as correct HC/LC pairing is critical to the assembly of 4-chain Hetero-Fcs, it was rationalized that parental mAbs showing high preference for cognate HC/LC pairing over non-cognate can serve as ideal building blocks. Simultaneously, those chains shown to be nonspecific (i.e. promiscuous LCs) present an opportunity to explore cLCs. Consequently, the deep understanding of the native properties of the antibody's sequences may facilitate the production of Bispecifics more amenable to its design goals.

### Description of Chain Selectivity Assessment Screening Methods

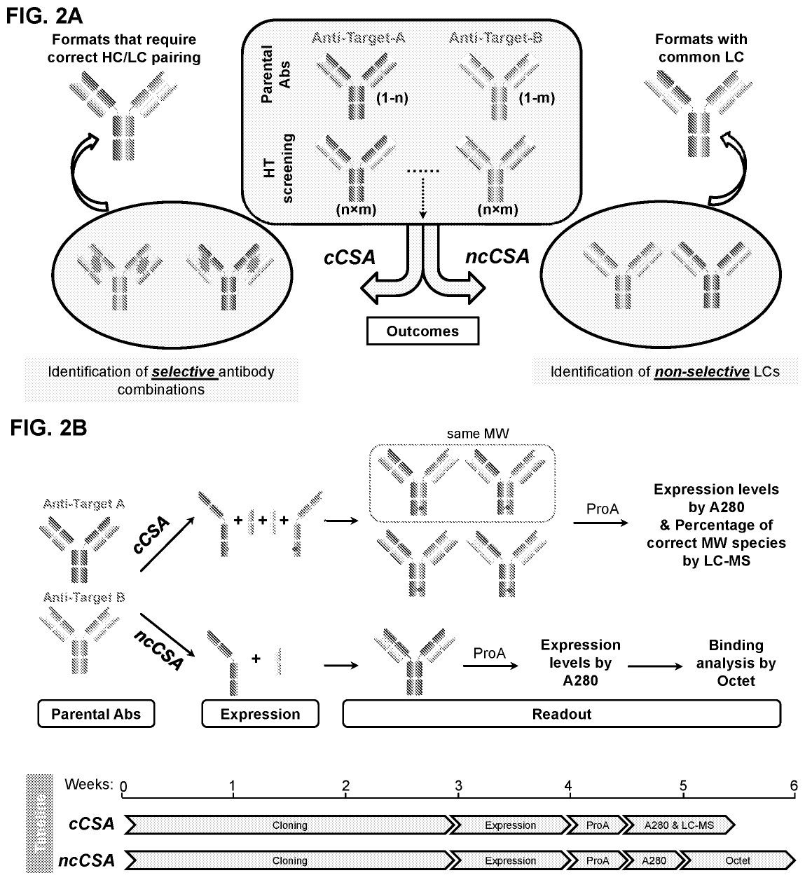

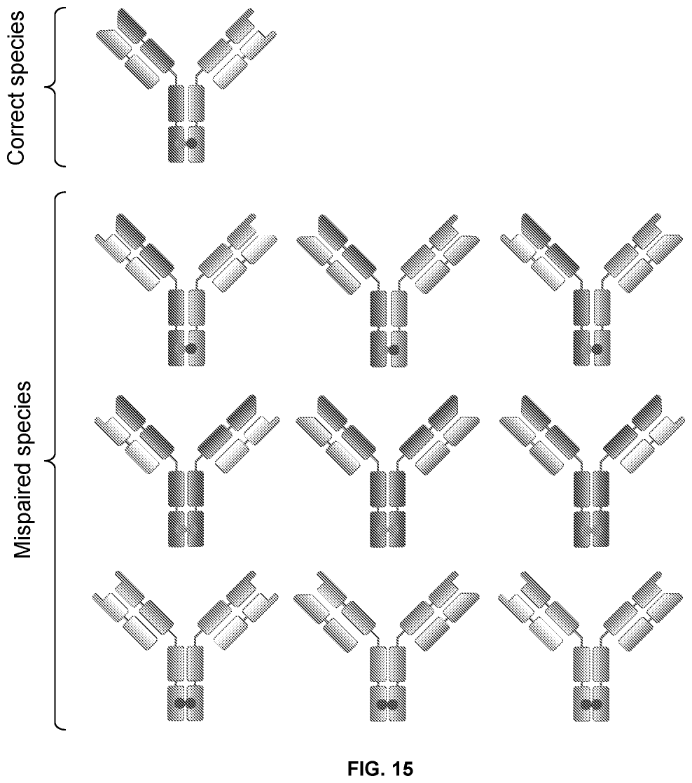

To facilitate the development of IgG-like Bispecifics, a high-throughput screening process, CSA, to evaluate HC/LC selectivity (FIG. 2A), is envisioned. Since the antibody HC is only secreted when bound to the LC^{21,22}, the measure of expression level of a given HC/LC pair may correlate to the HC/LC pairing efficiency. Starting from 2 panels of parental antibodies (anti-Target-A and anti-Target-B), high-throughput CSA was deployed in 2 different scenarios (competition and non-competition) to evaluate the specificity in the assembly of Hetero-Fcs (4 chains) and to identify promiscuous LCs for cLC Hetero-Fcs (3 chains), respectively (FIGS. 2A and B). The cCSA experiment mimics a co-expression scenario of 4 different chains (2×HCs and 2×LCs), resulting in all possible combinations between anti-Target-A and anti-Target-B parental antibodies. The expressed Hetero-Fcs were purified from conditioned medium with ProA beads and the percentage of correct HC/LC pairing was quantitated through high-resolution liquid chromatography-mass spectrometry (LC-MS) (FIGS. 2B and 15). It's worth noting that 2 out the 4 Hetero-Fc species may show the same MW, with one of them representing a scenario where both LCs (LCA and LCB) have a perfect cross pair with the opposite non-cognate HCs (FIG. 2B). Although this LC-MS cannot distinguish such species, this scramble of LCs is rarely seen even after limited proteolysis, strongly suggesting that the correct MW detected by LC-MS indicates correct HC/LC pairing. The high percentage of correct species is an indication of the preference for cognate HC/LC pairing over the non-cognate. The antibody combinations with high levels of correct species will be selected as the optimal building blocks for Bispecifics that require correct/specific HC/LC pairing (FIG. 2B). As for the ncCSA experiment, every single HC from anti-Target-A pairs with every LC from anti-Target-B at a 1:1 ratio, and vice versa. High expression levels (comparable to HC/LC cognate pair or higher) measured by ProA capture will indicate that LCs pair well and allow the non-cognate HCs to fold and to be secreted (FIG. 2B). In contrast, low expression levels suggest that the VH/VL interface of these chain(s) are specific to their cognate interfaces and as result cannot accommodate others. Next, using the same ProA purified material, a second high-throughput step was integrated to characterize the binding affinity using ForteBio Octet. This protocol enables a quick (less than 5 weeks) and efficient tool to screen and identify rare and valuable LCs that can be used as building blocks for cLC bispecific Abs.

### Identification of Low-Crosspairing Antibody Combinations with Competition CSA Method

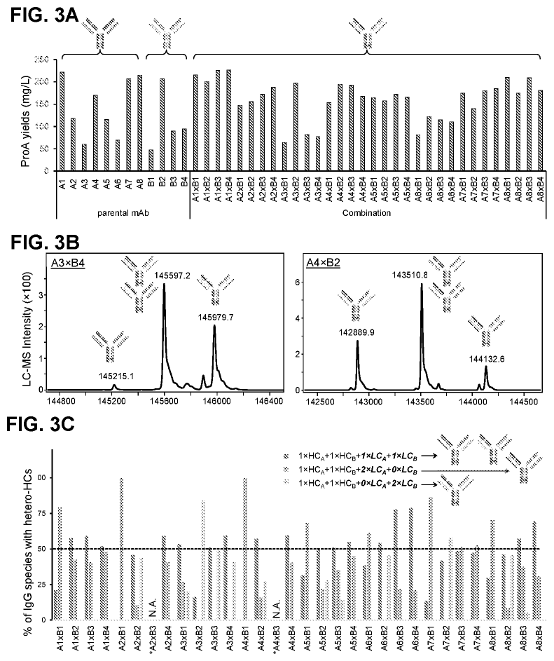

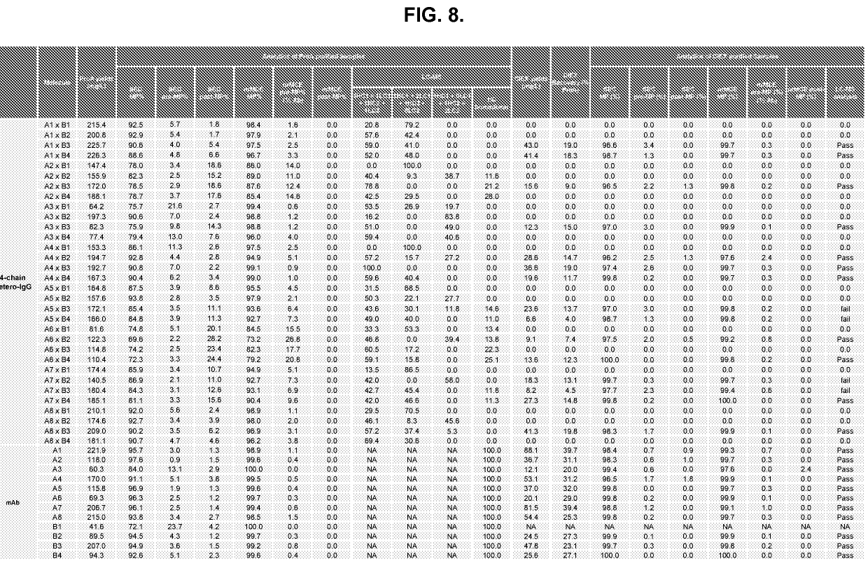

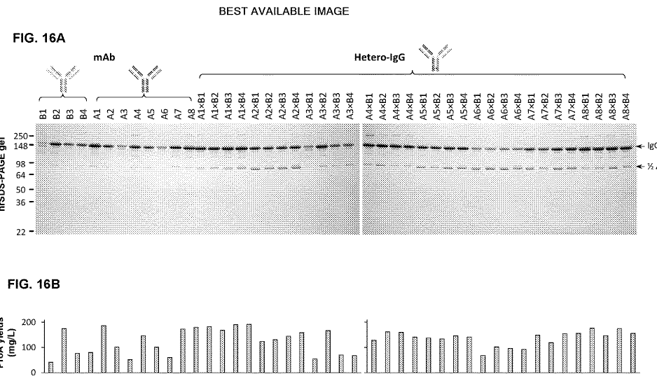

To validate the cCSA method, 2 panels of parental antibodies, 8× anti-Target-A Abs (A1-A8) and 4× anti-Target-B (B1-B4), were selected. Mutations were introduced to drive Heavy-Heavy chain pairing and combined antibodies from each target, resulting in 32 combinations for evaluation. Following the purification of the secreted IgGs from conditioned media, the expression levels of the combinations and parental mAbs were measured by A280 (FIGS. 3A and 16). Notably, parental mAbs demonstrated a high variation in expression levels ranging from 50 to 250 mg/L. Surprisingly, most of the antibody combinations expressed at levels higher than 100 mg/L with few exceptions (A3×B1, A3×B3, A3×B4 and A6×B1), which may be related to the low expressing parents A3 and B1. To identify whether the species assembled and secreted have correct HC/LC pairing, non-reducing LC-MS was used to analyze the ProA purified proteins (exemplified in FIG. 3B). By using the Fc region of the molecules for purification with ProA beads, HC containing molecules only was selected for and all other species (e.g. LC dimers) are discarded. Although the LC-MS analysis cannot confirm whether the 2 pairs of cognate HC/LC are correctly paired, it can determine whether each species has a copy of each of the 4 chains. While not definitive, this is a required condition towards the assembly and production of Hetero-Fcs. Therefore, the percentage for each of these 3 possible HC/LC scenarios was calculated: 1×HC_{A}+1×HC_{B}+1×LC_{A}+1×LC_{B}, 1×HC_{A}+1×HC_{B}+2×LC_{A}+0×LC_{B} and 1×HC_{A}+1×HC_{B}+0×LC_{A}+2×LC_{B}, all containing HC heterodimers (FIG. 3C). Although two combinations (A2×B3 and A4×B3) failed in LC-MS analysis due to small MW difference between two LCs (<60 Da), successful quantitation of the IgG species in all other 30 combinations was achieved. Furthermore, although Fc CPMs in the Ab combinations was deployed to enhance HC heterodimerization, there were still small amounts of homodimers and/or ½ Abs (FIG. 8). However, since the focus of this study is on HC/LC pairing, those species were excluded from further analysis. Interestingly, the data showed that in about half of the molecules tested (17/32), the percentage of the desired species (1×HC_{A}+1×LC_{A}+1×HC_{B}+1×LC_{B}) detected was ≥50% of the total species present in the ProA purified samples (FIG. 3C). Surprisingly, in 2 combinations (A6×B3 and A6×B4) the percentage of the correct product was >75%. In contrast, the ProA purified samples of the remaining 13 combinations contained <50% of the species of interest, with 5 of those combinations (A1×B1, A2×B1, A3×B2, A4×B1 and A7×B1) containing less than 25% of the desired species (FIG. 3C). In most cases (23/32) only 1/2 LCs appeared in the undesired product (1×HC_{A}+1×HC_{B}+2×LC_{A}+0×LC_{B} and 1×HC_{A}+1×HC_{B}+0×LC_{A}+2×LC_{B}), suggesting that either one of the LCs is overexpressing or that LC_{A} and LC_{B} may compete during expression and molecule assembly leading to a complete or partial suppression of the other (FIG. 3C). Thus, with this information, the cCSA method can efficiently screen and identify suitable combinations of parental mAbs with native properties that enable the correct assembly of the Hetero-Fcs upon expression in a single cell. This allows for deselection of candidates while directing efforts towards those with more promising characteristics, resulting in time and resources savings.

### Low-Crosspairing Antibody Combinations are Good Candidates for Making 4-Chain Hetero-Fcs

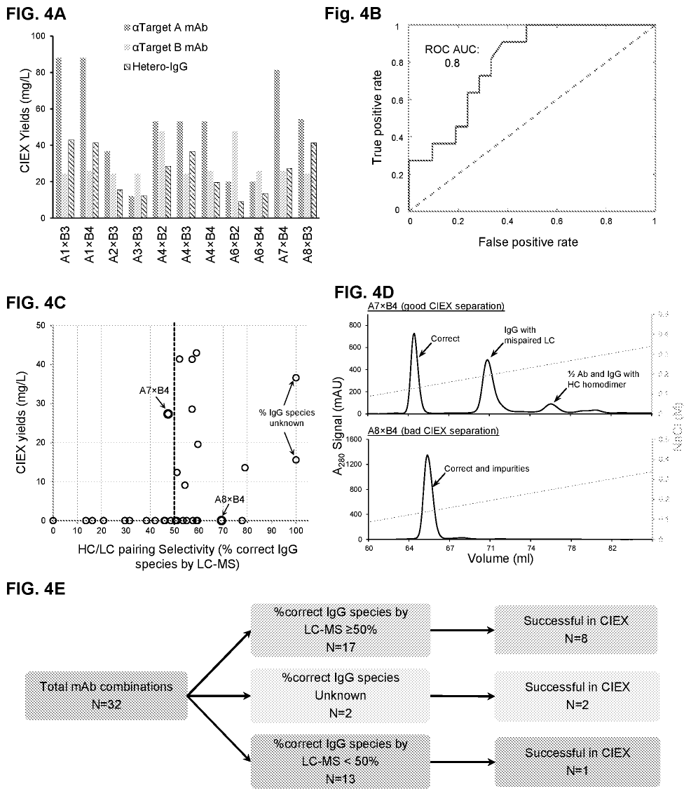

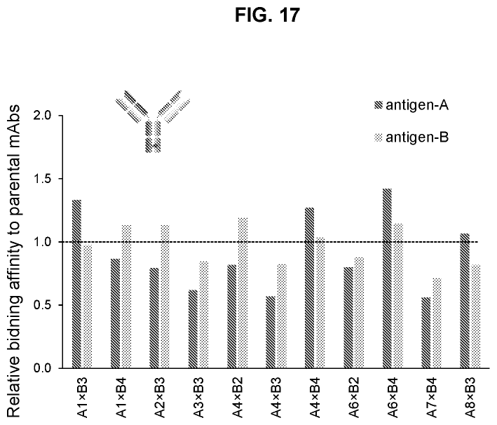

In the cCSA method described above, more than half (17/32) of the combinations showed high level (≥50%) of desired species (1×HC_{A}+1×LC_{A}+1×HC_{B}+1×LC_{B}) (FIG. 3C). However, to assess whether this high-throughput method was truly predictive of correct cognate HC/LC pairing, it was decided to scale-up the 32 Hetero-Fcs and perform a 2-step purification with ProA followed by a cation exchange chromatography (CIEX). The goal was not only to quantify the final yields of the desired species but also to study the separation profile in the CIEX step. From the 32 Hetero-Fcs, 11 were successfully purified with a final purity >90% as determined by SEC and LC-MS (FIG. 4A and FIG. 8). Interestingly, the final yields for the parental mAbs, the building blocks for these Hetero-Fcs, appear to correlate with the yields of the resulting bispecific molecules. Indeed, Hetero-Fcs A1×B3, A1×B4, A4×B3 and A8×B3 displaying the highest protein yields all contain at least one of the high expressing parental mAbs A1, A4 and A8 (FIG. 4A). For final verification, the final pools with LC-MS purity >90% were evaluated for binding against Target-A and —B, critical to evaluate the cognate HC/LC pairing. The affinity of each arm in the bispecific molecule to its respective target was comparable to that of the parent mAbs (FIG. 17), confirming that all 11 Hetero-Fcs have correct HC/LC in both arms.

Overall, this data aligns well with the cCSA experiment (FIG. 4E). From the 11 successfully purified Hetero-Fcs, the percentage of correct HC/LC pairing predicted by LC-MS was high for 8 molecules (≥50% correct HC/LC species, A1×B3, A1×B4, A3×B3, A4×B2, A4×B4, A6×B2, A6×B4 and A8×1B3), good for A7×B4 (47.4% correct species) and unknown for A2×B3 and A4×B3 (indeterminate due to the small MW difference between chains). Most importantly, none of the molecules that showed low HC/LC pairing by CSA could be purified by CIEX (FIGS. 3C and 4A). Indeed, the percentage of correct species determined by cCSA was predictive of which Ab combinations resulted in successful Hetero-Fcs (ROC AUC 0.80, FIG. 4B). Furthermore, we also attempted to build correlation between the percentage of HC/LC pairing predicted by cCSA and the final yields. As shown in FIG. 4C, most of the combinations (12/13) with <50% correct HC/LC pairing by cCSA failed later during the CIEX purification. One exception was the A7×B4 molecule that while displaying 47.4% pairing, it showed an CIEX profile with suitable separation (FIG. 4D). Interestingly, CIEX could not purify 9 out of 17 molecules with >50% correct HC/LC pairing predicted by cCSA (FIG. 4C). Indeed, to illustrate this phenomenon we have selected the example of the A8×B4 Hetero-Fc that displayed 69.4% correct pairing in the cCSA experiments. This molecule exhibits a well-shaped peak that conceals the mispaired species in such a way that no resolution between the two different species (1×HC_{A}+1×HC_{B}+1×LC_{A}+1×LC_{B} and 1×HC_{A}+1×HC_{B}+2×LC_{A}+0×LC_{B}) is observed (FIG. 4D). Thus, while the cCSA method can predict high performing Hetero-Fc molecules based on the native VH/VL interface, properties not screened by cCSA (such as those influencing separation profiles on ion-exchange chromatography columns) also play a significant role in selecting Hetero-Fcs.

### Screening for Common LCs with the Non-Competition CSA Method

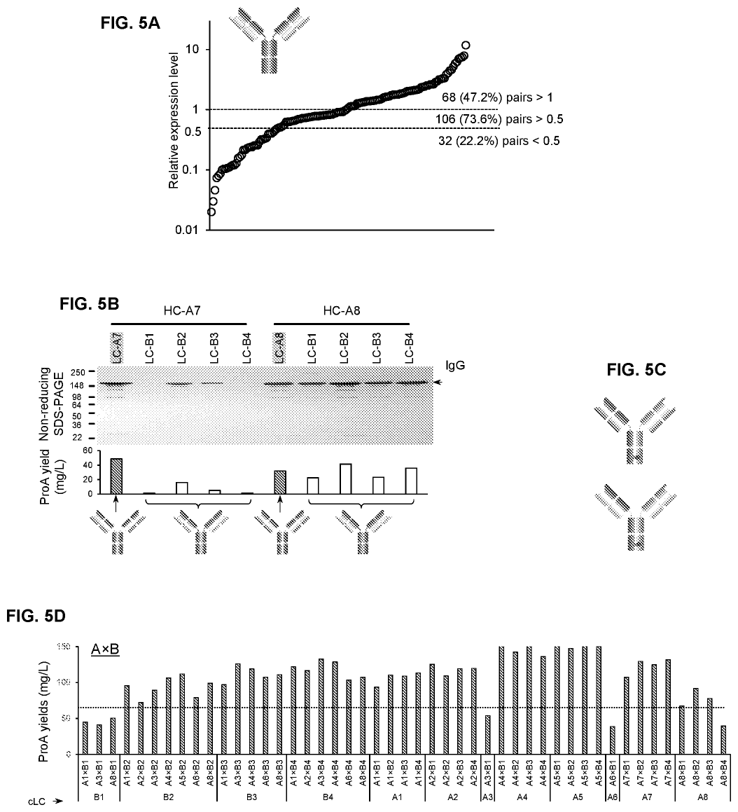

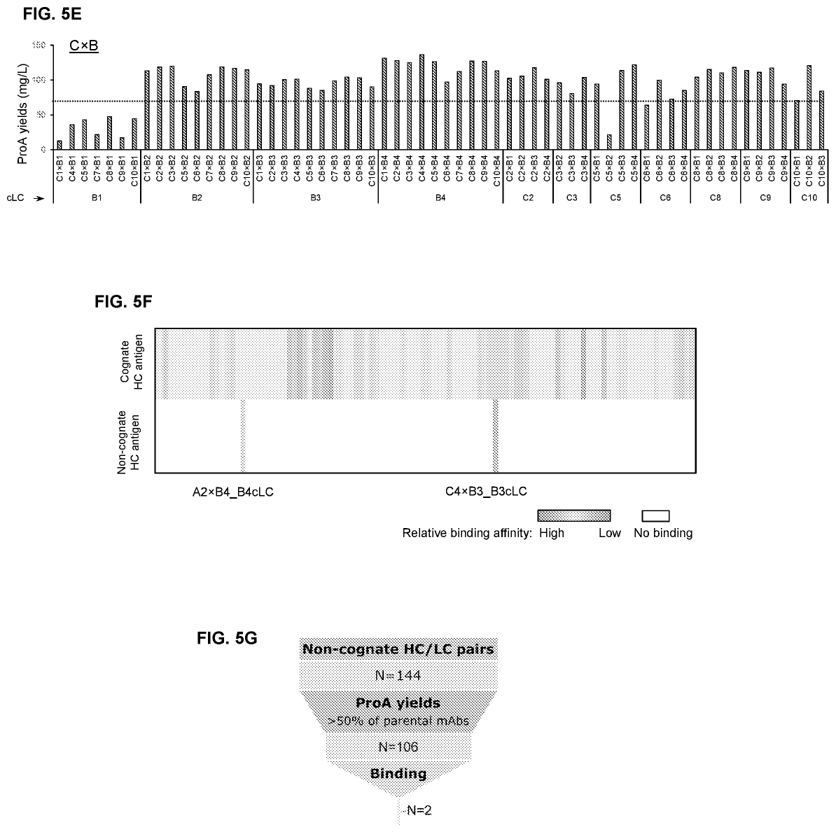

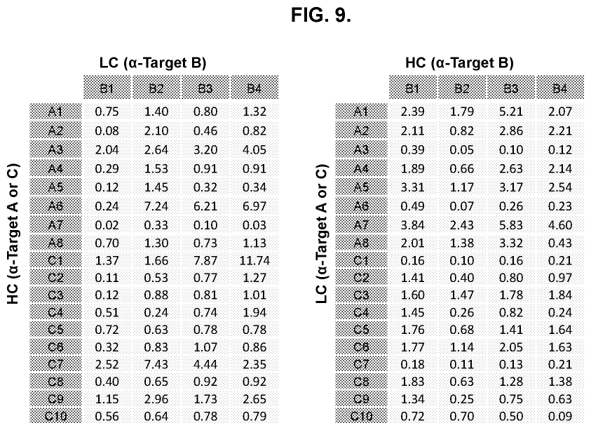

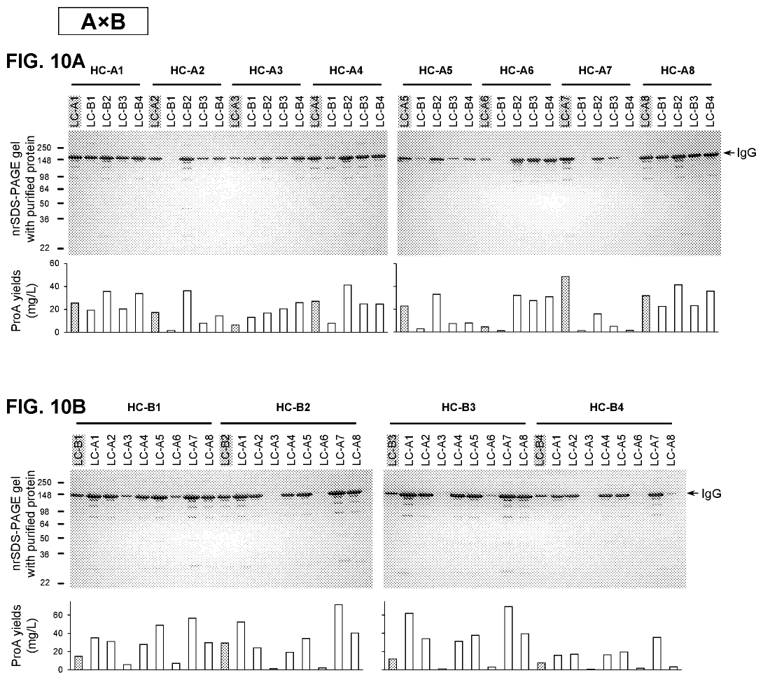

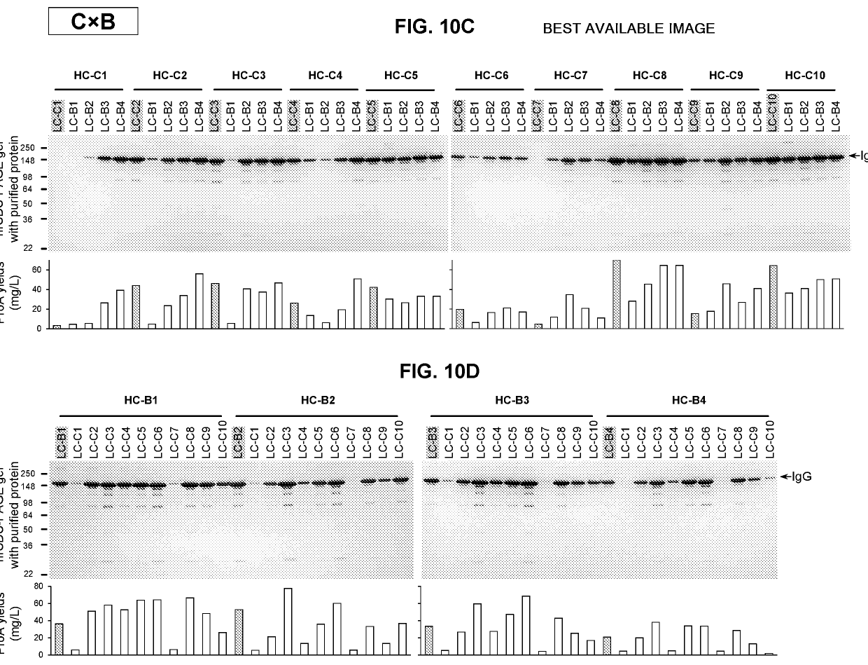

As shown above, while some LCs pair preferably with their cognate HCs, other LCs can also bind to non-cognate HCs efficiently. If the resulting non-cognate HC/LC pair retains binding, such a LC could serve as a common LC (cLC). The ncCSA method is envisioned as an opportunistic approach to evaluate whether the parental mAbs offer such LCs (FIG. 2B). To demonstrate the efficacy of this method, two bispecific programs (A×B and C×B) with 8 anti-Target-A, 4 anti-Target-B and 10 anti-Target-C parental mAbs were selected. As shown in FIG. 9, every LC of anti-Target-B was paired with individual HCs from anti-Target-A or -C mAbs. Meanwhile, each HC of anti-Target-B was combined with individual LCs from different anti-Target-A or -C mAbs. The resulting 144 non-cognate HC/LC pairs were expressed together with 22 parental mAbs (control) in 293 6E and purified with ProA beads, followed by analysis with non-reducing SDS PAGE gel and A280 quantitation (FIG. 10). Further analysis of the ProA yields for these 144 non-cognate HC/LC pairs (FIG. 5A and FIG. 9) showed that only 38 of the 144 combinations (26.4%) displayed a significant reduction in expression levels (50% or lower relative to the controls), suggesting an overall widespread promiscuous behavior within HC/LC pairing. Of the remaining 106 combinations, 68 (47.2%) HC/LC non-cognate pairing molecules displayed higher expression levels than the corresponding parental mAb controls (FIG. 5A). However, many LCs were not broadly promiscuous. A closer look into 2 examples is useful to illustrate this phenomenon. When LCs-B1-4 were paired with HC-A7, protein expression was significantly lower when compared to the cognate LC-A7 (control) (FIG. 5B). In contrast, the expression levels of the same anti-Target-B LCs while paired with HC-A8 are comparable or higher than cognate LC-A8 (control). Thus, suggesting that the anti-Target-B LCs are promiscuous with respect to HC-A8 but not towards HC-A7, highlighting the role that HCs also play a role in determining LC cross pairing.

### Binding Analysis Identified Common LC Hetero-Fc Candidates

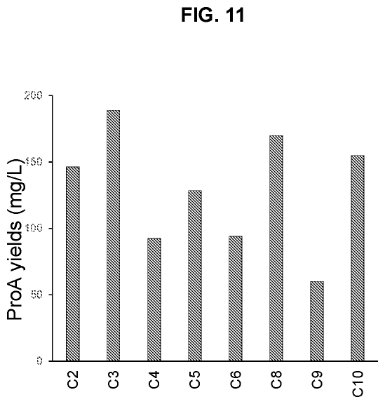

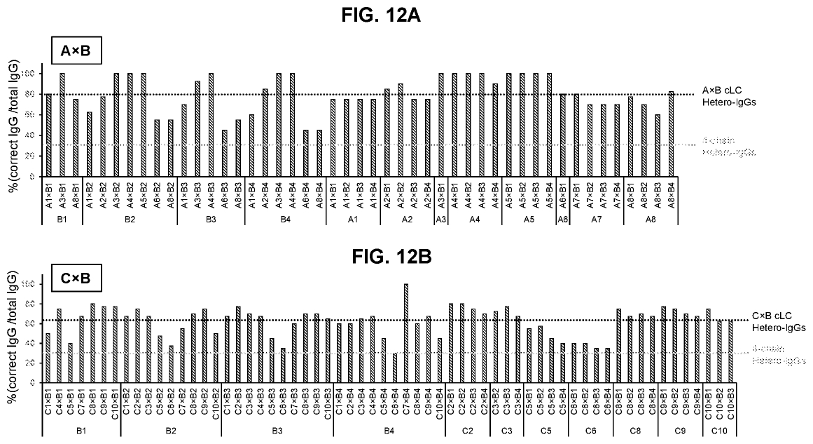

Although well-expressing non-cognate Ab combinations are promising candidates to assemble cLC Bispecifics, expression levels alone provide no insight into function. To select for functional Bispecifics, selected promiscuous LCs (demonstrating 50% or higher expression levels relative to cognate controls during the ncCSA assay (FIG. 5A)) were assembled into a Hetero-Fc format with both the cognate and non-cognate HCs, containing Fc CPMs to promote HC dimerization (FIG. 5C). These cLC Hetero-Fcs were evaluated with a high-throughput binding assay to identify candidates that allow for binding to both targets. After high-throughput expression in 4 mL deep well blocks (DWB), with HEK293-6E cells, the cLC Hetero-Fcs were purified by ProA. The yields for 92 out of 106 of the molecules (86.8%), was ˜100 mg/L, which is comparable to the parental mAbs (FIG. 3A and FIG. 11), with only 14 cLC Hetero-Fcs (13.2%) showing ProA yields lower than 60 mg/L (FIGS. 5D and E). All molecules with cLC B1 showed a remarkably lower expression, suggesting that this LC may act as limiting factor in the overall expression of these Bispecifics (FIG. 5D). Indeed, the ProA yield for B1 Ab with 41.6 mg/L was the lowest among all parental mAbs used as building blocks for the generation of these Bispecifics (FIG. 3A). Although A3 and A6 parental mAbs also showed a relatively low ProA recovery (60.3 and 69.3 mg/L, respectively), the cLC Hetero-Fcs containing these 2 building blocks showed acceptable protein yields when combined with any B parental but B1, suggesting that in this case the non-cognate HCs may have rescued the expression levels (FIG. 5D). Another important observation is that the cLC Hetero-Fcs (A×B and C×B) also showed a ˜2-fold increase overall in correct pairing over the 4-chain Hetero-Fcs just after ProA purification, highlighting the impact of HC/LC pairing in the production levels of these molecules (FIG. 12).

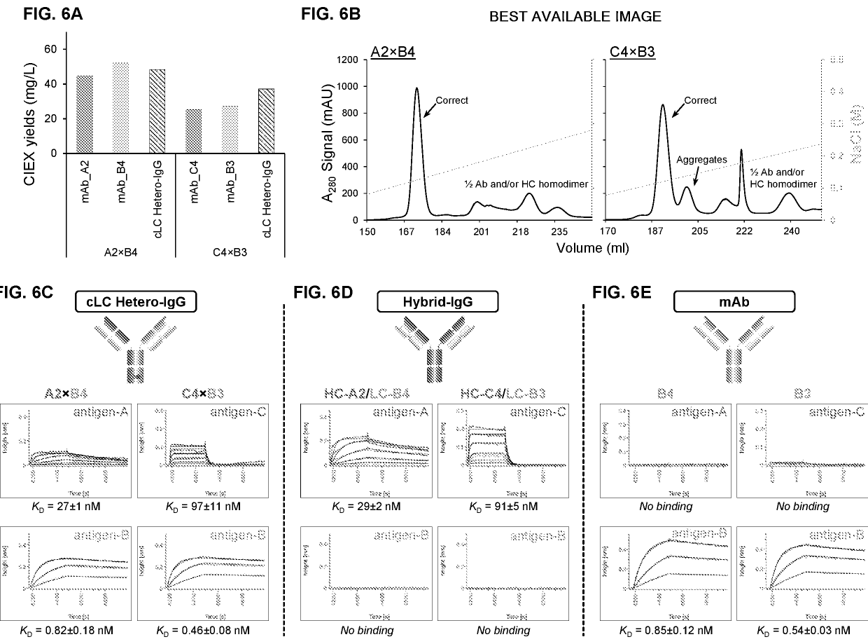

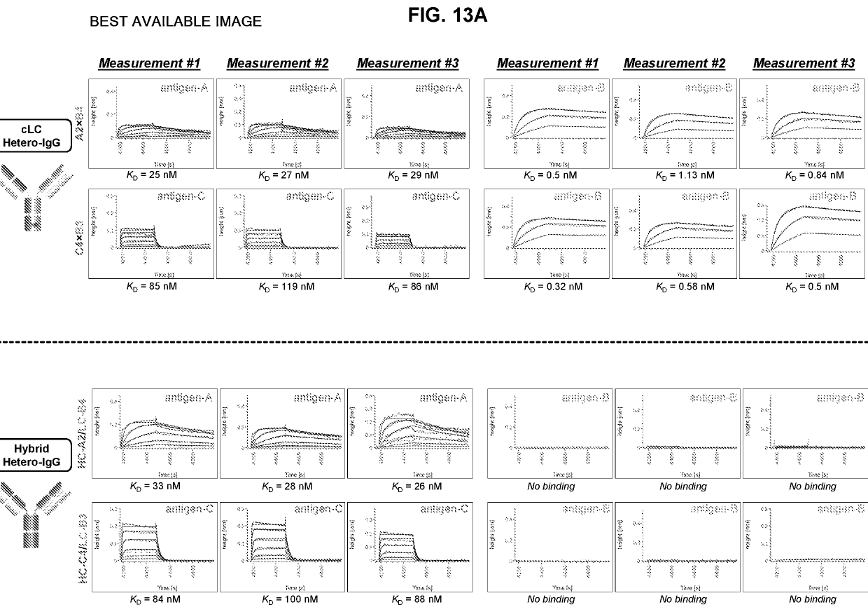

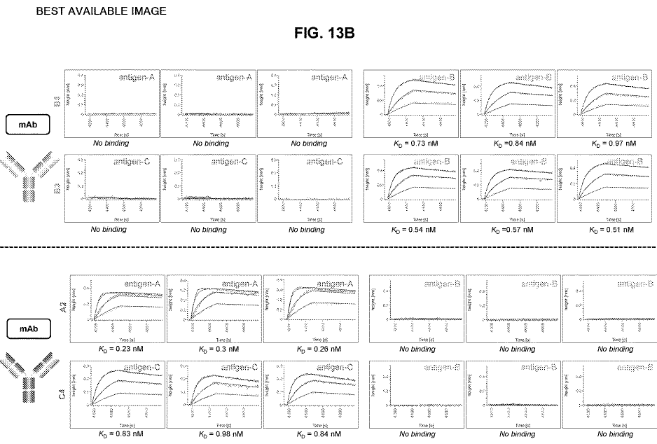

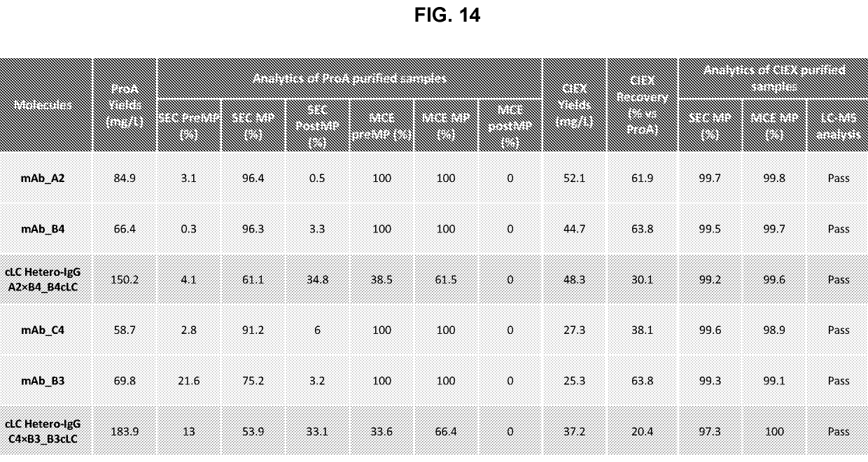

For rapid binding screening, these single-step purified samples were then assessed by ForteBio Octet. To minimize interference by residual impurities, the cLC-Hetero-Fc molecules were first captured onto Streptavidin fiber optic biosensors with a biotinylated anti-human IgG Fc polyclonal antibody via the Fc region and soluble antigen-A, -B or -C were loaded for incubation. As expected, all cLC Hetero-Fcs displayed binding to their respective targets via the cognate HC/LC arm, with comparable affinity to the parental mAbs (FIG. 5F). 2 cLC Hetero-Fcs (A2×B4 and C4×B3), also showed detectable binding via the non-cognate HC/LC arm recognizing Target-A or -C(FIG. 5F). In the case of A2×B4, the B4 LC was paired with both HCs (A2 and B4), whereas for C4×B3 the HCs (C4 and B3) were both paired with the B3 LC. Of note, these cLCs were both generated against Target-B. Although this non-canonical binding is lower than the single-digit nM binding typically observed for parental mAbs (FIG. 13), it demonstrates how the ncCSA method provides a new opportunity to identify LCs with unique structural features allowing for highly efficient pairing with non-cognate HCs (FIG. 5G). Furthermore, rapid binding analysis can reveal those rare cLCs that also retain binding to a new epitope. Since the manufacturability of IgG-like Bispecifics is often challenging with production levels below that of mAbs²³, the expression and purification properties of these cLC Hetero-Fcs was explored. To better mimic the scale and purification process required for therapeutic candidates, these 2 molecules were expressed in 250 mL HEK293-6E cells and subjected to a 2-step purification with ProA followed by CIEX to meet the purity target of >95%. Notably, the levels of protein secretion, by ProA, were over 2-fold higher for these 2 cLC Hetero-Fcs when compared to the parental mAbs (FIG. 14). More importantly, these cLC Hetero-Fcs showed a final yield comparable to or higher than the parental mAbs (FIG. 6A), all with over 97% purity of the desired species (FIG. 14). Moreover, these Bispecifics showed favorable CIEX profiles with correct species easily separated from the impurities (FIG. 6B). The binding assay was then repeated using the fully purified cLC Hetero-Fcs, to confirm their affinity for the respective antigens. As observed initially (FIG. 5F), these 2 molecules showed binding affinity via their non-cognate HC/LC arms to antigen-A or C while retaining the binding properties in the cognate arms to antigen-B (FIGS. 6C and S7). To validate the affinity measured for these cLC Hetero-Fcs, two hybrid IgGs composed of a non-cognate HC and LC each (HC-A2/LC-B4 and HC-C4/LC-B3) were expressed and purified. The comparable affinities of the hybrid molecules to antigen-A and —C via their non-cognate arms (FIG. 6D), further confirmed the cLC Hetero-Fcs binding. The binding signal for the hybrid IgGs was ˜2-fold higher than the signal observed for the non-cognate arm in the cLC Hetero-Fcs, which agrees with the number of binding sites present in these molecules (2 vs 1, respectively). Moreover, since neither of them seems to retain binding to antigen-B, it suggests that the binding capability of hybrid IgGs is mostly driven by HC CDRs but not LC. Inversely, to also exclude the possibility of non-specific binding to antigen-A or —C by the cognate arms in the cLC Hetero-Fcs, the binding for B4 and B3 parental mAbs was tested. As shown in FIG. 6E, B4 and B3 mAbs did not bind to these antigens, further demonstrating the binding detected for the non-cognate arm is not derived from a non-specific interaction between cognate arm and antigen-A or -C nor is the result of cLC alone.

Altogether, the ncCSA method successfully identified two rare LCs from a pool of mAbs that paired with non-cognate HCs showing mAb like productivity while allowing the HC in this non-cognate HC/LC arm to retain binding activity.

All references cited within the present application are hereby incorporated by reference.
1. Labrijn, A. F., Janmaat, M. L., Reichert, J. M. & Parren, P. Bispecific antibodies: a mechanistic review of the pipeline. Nat Rev Drug Discov 18, 585-608 (2019).
2. Lu, R. M. et al. Development of therapeutic antibodies for the treatment of diseases. J Biomed Sci 27, 1 (2020).
3. Fan, G., Wang, Z., Hao, M. & Li, J. Bispecific antibodies and their applications. J Hematol Oncol 8, 130 (2015).
4. Wolf, E., Hofmeister, R., Kufer, P., Schlereth, B. & Baeuerle, P. A. BiTEs: bispecific antibody constructs with unique anti-tumor activity. Drug Discov Today 10, 1237-1244 (2005).
5. Kantarjian, H. et al. Blinatumomab versus Chemotherapy for Advanced Acute Lymphoblastic Leukemia. N Engl J Med 376, 836-847 (2017).
6. Brinkmann, U. & Kontermann, R. E. The making of bispecific antibodies. MAbs 9, 182-212 (2017).
7. Wang, Q. et al. Design and Production of Bispecific Antibodies. Antibodies (Basel) 8 (2019).
8. Spiess, C., Zhai, Q. & Carter, P. J. Alternative molecular formats and therapeutic applications for bispecific antibodies. Mol Immunol 67, 95-106 (2015).
9. Suresh, M. R., Cuello, A. C. & Milstein, C. Bispecific monoclonal antibodies from hybrid hybridomas. Methods Enzymol 121, 210-228 (1986).
10. Carter, P. Bispecific human IgG by design. J Immunol Methods 248, 7-15 (2001).
11. Ridgway, J. B., Presta, L. G. & Carter, P. ‘Knobs-into-holes’ engineering of antibody CH3 domains for heavy chain heterodimerization. Protein Eng 9, 617-621 (1996).
12. Davis, J. H. et al. SEEDbodies: fusion proteins based on strand-exchange engineered domain (SEED) CH3 heterodimers in an Fc analogue platform for asymmetric binders or immunofusions and bispecific antibodies. Protein Eng Des Sel 23, 195-202 (2010).
13. Gunasekaran, K. et al. Enhancing antibody Fc heterodimer formation through electrostatic steering effects: applications to bispecific molecules and monovalent IgG. J Biol Chem 285, 19637-19646 (2010).
14. Dillon, M. et al. Efficient production of bispecific IgG of different isotypes and species of origin in single mammalian cells. MAbs 9, 213-230 (2017).
15. Shiraiwa, H. et al. Engineering a bispecific antibody with a common light chain: Identification and optimization of an anti-CD3 epsilon and anti-GPC3 bispecific antibody, ERY974. Methods 154, 10-20 (2019).
16. Merchant, A. M. et al. An efficient route to human bispecific IgG. Nat Biotechnol 16, 677-681 (1998).
17. Krah, S. et al. Generation of human bispecific common light chain antibodies by combining animal immunization and yeast display. Protein Eng Des Sel 30, 291-301 (2017).
18. Garces, F. et al. Molecular Insight into Recognition of the CGRPR Complex by Migraine Prevention Therapy Aimovig (Erenumab). Cell Rep 30, 1714-1723 e1716 (2020).
19. Bruggemann, M. et al. Human antibody production in transgenic animals. Arch Immunol Ther Exp (Warsz) 63, 101-108 (2015).
20. Foltz, I. N., Gunasekaran, K. & King, C. T. Discovery and bio-optimization of human antibody therapeutics using the XenoMouse(R) transgenic mouse platform. Immunol Rev 270, 51-64 (2016).
21. Feige, M. J. et al. An unfolded CH1 domain controls the assembly and secretion of IgG antibodies. Mol Cell 34, 569-579 (2009).
22. Feige, M. J. & Buchner, J. Principles and engineering of antibody folding and assembly. Biochim Biophys Acta 1844, 2024-2031 (2014).
23. Qiong Wang, Y. C., Jaeyoung Park, Xiao Liu, Yifeng Hu, Tiexin Wang, & Betenbaugh, K. M.a.M. J. Design and Production of Bispecific Antibodies. Antibodies (2019).
24. Schaefer, W. et al. Immunoglobulin domain crossover as a generic approach for the production of bispecific IgG antibodies. Proc Natl Acad Sci USA 108, 11187-11192 (2011).
25. Van Blarcom, T. et al. Productive common light chain libraries yield diverse panels of high affinity bispecific antibodies. MAbs 10, 256-268 (2018).
26. Feige, M. J., Hendershot, L. M. & Buchner, J. How antibodies fold. Trends Biochem Sci 35, 189-198 (2010).
27. Saphire, E. O. et al. Crystal structure of a neutralizing human IGG against HIV-1: a template for vaccine design. Science 293, 1155-1159 (2001).
28. Scapin, G. et al. Structure of full-length human anti-PD1 therapeutic IgG4 antibody pembrolizumab. Nat Struct Mol Biol 22, 953-958 (2015).
29. Krissinel, E. & Henrick, K. Inference of macromolecular assemblies from crystalline state. J Mol Biol 372, 774-797 (2007).
30. Engler, C., Kandzia, R. & Marillonnet, S. A one pot, one step, precision cloning method with high throughput capability. PLoS One 3, e3647 (2008).
31. Jacobsen, F. W. et al. Engineering an IgG Scaffold Lacking Effector Function with Optimized Developability. J Biol Chem 292, 1865-1875 (2017).
32. Yoo, D., Provchy, J., Park, C., Schulz, C. & Walker, K. Automated high-throughput protein purification using an AKTApurifier and a CETAC autosampler. J Chromatogr A 1344, 23-30 (2014).
33. Winters, D., Chu, C. & Walker, K. Automated two-step chromatography using an AKTA equipped with in-line dilution capability. J Chromatogr A 1424, 51-58 (2015).
34. Spahr, C. S. et al. Discovery, characterization, and remediation of a C-terminal Fc-extension in proteins expressed in CHO cells. MAbs 10, 1291-1300 (2018).

We claim:

1. A method for selecting a multispecific antibody construct, the method comprising:
(a) obtaining a plurality of antibody Fab fragments, scFvs, or a combination thereof wherein each Fab fragment and scFv comprises three heavy chain CDRs and three light chain CDRs that specifically bind a first antigen;
(b) obtaining a plurality of antibody Fab fragments, scFvs, or a combination thereof wherein each Fab fragment and scFv comprises three heavy chain CDRs and three light chain CDRs that specifically bind a second antigen;
(c) cloning into a vector:
(i) the CDRs of the three heavy chain CDRs that specifically bind the first antigen,
(ii) the CDRs of the three heavy chain CDRs that specifically bind the second antigen, and
(iii) three light chain CDRs selected from the group consisting of
(a) the CDRs of the three light chain CDRs that specifically bind the first antigen,
(b) the CDRs of the three light chain CDRs that specifically bind the second antigen, and
(c) CDRs of three light chain CDRs that do not specifically bind either the first antigen or the second antigen;
wherein the vector(s) encode(s) for one type of multispecific antibody construct module and a plurality of vectors are generated that encode for a plurality of multispecific antibody constructs comprising the heavy chain CDRs that bind to each antigen and the light chain CDRs of (c)(iii);
(d) expressing each multispecific antibody construct in a mammalian host cell;
(e) purifying each multispecific antibody construct;
(f)(i) measuring the expression levels of each multispecific antibody construct and (ii) measuring the binding affinity of each multispecific antibody construct to the first antigen and the second antigen, wherein steps (f)(i) and (f)(ii) can be performed simultaneously or in any order; and
(g) comparing the expression levels of (f)(i) and the binding affinities of (f)(ii) for each multispecific antibody construct in order to identify the optimal pairing of the three heavy chain CDRs that specifically bind a first antigen and the light chain CDRs of (c)(iii) with the optimal pairing of the three heavy chain CDRs that specifically bind a second antigen and the same light chain CDRs of (c)(iii).

2. The method according to claim 1, wherein the plurality of antibody Fab fragments, scFvs, or a combination thereof, wherein each Fab fragment and scFv comprise three heavy chain CDRs and three light chain CDRs that specifically bind a first antigen is selected from the group consisting of at least two antibody Fab fragments, scFvs, or a combination thereof: at least three antibody Fab fragments, scFvs, or a combination thereof; at least four antibody Fab fragments, scFvs, or a combination thereof; at least five antibody Fab fragments, scFvs, or a combination thereof: at least six antibody Fab fragments, scFvs, or a combination thereof; at least seven antibody Fab fragments, scFvs, or a combination thereof at least eight antibody Fab fragments, scFvs, or a combination thereof; at least nine antibody Fab fragments, scFvs, or a combination thereof; and at least ten antibody Fab fragments, scFvs, or a combination thereof.

3. The method according to claim 1, wherein the plurality of antibody Fab fragments, scFvs, or a combination thereof, wherein each Fab fragment and scFv comprise three heavy chain CDRs and three light chain CDRs that specifically bind a second antigen is selected from the group consisting of at least two antibody Fab fragments, scFvs, or a combination thereof; at least three antibody Fab fragments, scFvs, or a combination thereof; at least four antibody Fab fragments, scFvs, or a combination thereof; at least five antibody Fab fragments, scFvs, or a combination thereof: at least six antibody Fab fragments, scFvs, or a combination thereof; at least seven antibody Fab fragments, scFvs, or a combination thereof; at least eight antibody Fab fragments, scFvs, or a combination thereof; at least nine antibody Fab fragments, scFvs, or a combination thereof; and at least ten antibody Fab fragments, scFvs, or a combination thereof.

4. The method according to claim 1, wherein the multispecific antibody construct modules include at least two of the modules selected from the group consisting of Fab/Fab hetero Fc, scFab/scFab hetero Fc, Fab/scFv hetero Fc, Fab/Fab-scFv hetero Fc, Fab/scFv-Fab hetero Fc, Fab/Fab hetero Fc-scFv, IgG-Fab, scFab-Fe-Fab, IgG-scFv, scFv-IgG, and Fab-scFv-Fc.

5. The method according to claim 1, wherein the mammalian host cell is selected from the group consisting of Chinese hamster ovary (“CHO”) cells, monkey kidney CV1 line transformed by SV40 (“COS-7”); human embryonic kidney line HEK293-6E (“HEK293-6E”); human embryonic kidney line 293 (“HEK93”)baby hamster kidney cells (“BHK”); mouse sertoli cells (“TM4”); monkey kidney cells (“CV1”): African green monkey kidney cells (“VERO-76”); human cervical carcinoma cells (“HELA”): canine kidney cells (“MDCK”); buffalo rat liver cells (“BRL”); human lung cells (“W138”); human hepatoma cells (“Hep G2”); mouse mammary tumor (“MMT”); TRI cells; MRC 5 cells: and FS4 cells.

6. The method according to claim 1, wherein the expression levels are determined by a method selected from the group consisting of A280 measurement SDS-PAGE, microchip capillary electrophoresis (MCE), Bradford assay, and bicinchoninic acid (BCA) assay.

7. The method according to claim 1, wherein binding affinity of each multispecific antibody construct to the first antigen and the second antigen is measured using Octet, Forte Bio, Carterra LSA, SPR and Flow cytometry.

8. The method according to claim 1, wherein each multispecific antibody construct is purified by Protein A, Lambda and Kappa resins, and affinity tag purification.

9. A method for selecting a multispecific antibody construct, the method comprising:
(a) obtaining a plurality of at least two antibody Fab fragments, scFvs, or a combination thereof, wherein each Fab fragment and scFv comprises three heavy chain CDRs and three light chain CDRs that specifically bind a first antigen;
(b) obtaining a plurality of at least two antibody Fab fragments, scFvs, or a combination thereof, wherein each Fab fragment and scFv comprises three heavy chain CDRs and three light chain CDRs that specifically bind a second antigen:
(c) cloning into a vector:
(i) the CDRs of the three heavy chain CDRs that specifically bind the first antigen,
(ii) the CDRs of the three heavy chain CDRs that specifically bind the second antigen, and
(iii) three light chain CDRs selected from the group consisting of
(a) the CDRs of the three light chain CDRs that specifically bind the first antigen,
(b) the CDRs of the three light chain CDRs that specifically bind the second antigen, and
(c) CDRs of three light chain CDRs that do not specifically either the first antigen or the second antigen;
wherein the vector(s) encode(s) for a multispecific antibody construct nodule and a plurality of vectors are generated that encode for a plurality of multispecific antibody constructs comprising the heavy chain CDRs that bind to each antigen and the light chain CDRs of (c)(iii);
(d) expressing each multispecific antibody construct in a mammalian host cell, wherein the mammalian host cell is selected from the group consisting of HEK293-6E cells and CHO cells;
(e) purifying each multispecific antibody construct using Protein A chromatography;
(f)(i) measuring the expression levels of each multispecific antibody construct using A280 measurement and (ii) measuring the binding affinity of each multispecific antibody construct to the first antigen and the second antigen using Octet,
wherein steps (f)(i) and (f)(ii) can be performed simultaneously or in any order; and
(g) comparing the expression levels of (f)(i) and the binding affinities of (f)(ii) for each multispecific antibody construct in order to identify the optimal pairing of the three heavy chain CDRs that specifically bind a first antigen and the light chain CDRs of (c)(iii) with the optimal pairing of the three heavy chain CDRs that specifically bind a second antigen and the same light chain CDRs of (c)(iii).

10. The method according to claim 9, wherein the multispecific antibody construct modules include at least two of the modules selected from the group consisting of Fab/Fab hetero Fc, scFab/scFab hetero Fc, Fab/scFv hetero Fc, Fab/Fab-scFv hetero Fc, Fab/scFv-Fab hetero Fc, Fab/Fab hetero Fc-scFv, IgG-Fab, scFab-Fc-Fab, IgG-scFv, scFv-IgG, and Fab-scFv-Fc.

11. A method for selecting a multispecific antibody construct, the method comprising:
(a) obtaining a plurality of antibody Fab fragments, scFvs, or a combination thereof, wherein each Fab fragment and scFv comprises three heavy chain CDRs and three light chain CDRs that specifically bind a first antigen;
(b) obtaining a plurality of antibody Fab fragments, scFvs, or a combination thereof, wherein each Fab fragment and scFv comprises three heavy chain CDRs and three light chain CDRs that specifically bind a second antigen;
(c) cloning the CDRs of the two pluralities into a vector(s) that encode(s) for a multispecific antibody construct module and a plurality of vectors are generated that encode a plurality of multispecific antibody constructs comprising the CDRs that bind to each antigen:
(d) expressing each multispecific antibody construct in a mammalian host cell;
(e) purifying each multispecific antibody construct;
(f)(i) measuring the expression levels of each multispecific antibody construct and (ii) calculating the percent of correct and incorrect multispecific antibody construct module species produced, wherein steps (f)(i) and (f)(ii) can be performed simultaneously or in any order; and
(g) comparing the expression levels of (f)(i) and the percentage of correct and incorrect multispecific antibody constrict module species of (f)(ii) for each multispecific antibody construct in order to identify the optimal pairing of three heavy chain CDRs and three light chain CDRs that specifically bind a first antigen with three heavy chain CDRs and three light chain CDRs that specifically bind a second antigen.

12. The method according to claim 11, wherein the plurality of antibody Fab fragments, scFvs, or a combination thereof, wherein each Fab fragment and scFv comprise three heavy chain CDRs and three light chain CDRs that specifically bind a first antigen is selected from the group consisting of at least two antibody Fab fragments, scFvs, or a combination thereof; at least three antibody Fab fragments, scFvs, or a combination thereof at least four antibody Fab fragments, scFvs, or a combination thereof; at least five antibody Fab fragments, scFvs, or a combination thereof; at least six antibody Fab fragments, scFvs, or a combination thereof; at least seven antibody Fab fragments, scFvs, or a combination thereof; at least eight antibody Fab fragments, scFvs, or a combination thereof; at least nine antibody Fab fragments, scFvs, or a combination thereof; and at least ten antibody Fab fragments, scFvs, or a combination thereof.

13. The method according to claim 11, wherein the plurality of antibody Fab fragments, scFvs, or a combination thereof, wherein each Fab fragment and scFv comprise three heavy chain CDRs and three light chain CDRs that specifically bind a second antigen is selected from the group consisting of at least two antibody Fab fragments, scFvs, or a combination thereof; at least three antibody Fab fragments, scFvs, or a combination thereof: at least four antibody Fab fragments, scFvs, or a combination thereof; at least five antibody Fab fragments, scFvs, or a combination thereof; at least six antibody Fab fragments, scFvs, or a combination thereof: at least seven antibody Fab fragments, scFvs, or a combination thereof; at least eight antibody Fab fragments, scFvs, or a combination thereof; at least nine antibody Fab fragments, scFvs, or a combination thereof; and at least ten antibody Fab fragments, scFvs, or a combination thereof.

14. The method according to claim 11, wherein the multispecific antibody construct module is selected from the group consisting of Fab/Fab hetero Fc, scFab/scFab hetero Fc, Fab/scFv hetero Fc, Fab/Fab-scFv hetero Fc, Fab/scFv-Fab hetero Fc, Fab/Fab hetero Fc-scFv, IgG-Fab, scFab-Fc-Fab, IgG-scFv, scFv-IgG, and Fab-scFv-Fc.

15. The method according to claim 11, wherein the mammalian host cell is selected from the group consisting of Chinese hamster ovary (“CHO”) cells, monkey kidney CV1 line transformed by SV40 (“COS-7”); human embryonic kidney line HEK293-6E (“HEK293-6E”); human embryonic kidney line 293 (“HEK293”); baby hamster kidney cells “BHK”); mouse sertoli cells (“TM4”): monkey kidney cells (“CV1”); African green monkey kidney cells (“VERO-76”); human cervical carcinoma cells (“HELA”); canine kidney cells (“MDCK”): buffalo rat liver cells (“BRL”); human lung cells (“W138”): human hepatoma cells (“Hep G2”); mouse mammary tumor (“MMT”); TRI cells; MRC 5 cells: and FS4 cells.

16. The method according to claim 1, wherein the expression levels are determined by a method selected from the group consisting of A280 measurement SDS-PAGE, microchip capillary electrophoresis (MCE), Bradford assay, aid bicinchoninic acid (BCA) assay.

17. The method according to claim 11, wherein the percentage of correct and incorrect multispecific antibody construct modality species is determined by a method selected from the group consisting of liquid chromatography-mass spectrometry (“LC-MS”), Caliper, HPLC SEC, SDS-PAGE, and microchip capillary electrophoresis (“MCE”).

18. The method according to claim 11, wherein each multispecific antibody construct is purified by Protein A, Lambda and Kappa resins, and affinity tag purification.

19. A method for selecting a multispecific antibody construct, the method comprising:
(a) obtaining a plurality of at least two antibody Fab fragments, scFvs, or a combination thereof, wherein each Fab fragment and scFv comprises three heavy chain CDRs and three light chain CDRs that specifically bind a first antigen:
(b) obtaining a plurality of at least two antibody Fab fragments, scFvs, or a combination thereof, wherein each Fab fragment and scFv comprises three heavy chain CDRs and three light chain CDRs that specifically bind a second antigen;
(c) cloning the CDRs of the two pluralities into a vector(s) that encode(s) for a multispecific antibody construct module and a plurality of vectors are generated that encode a plurality of multispecific antibody constructs that bind to each antigen;
(d) expressing each multispecific antibody construct in a mammalian host cell, wherein the mammalian host cell is selected from the group consisting of HEK293-6E cells and CHO cells;
(e) purifying each multispecific antibody construct using Protein A chromatography;
(f)(i) measuring the expression levels of each multispecific, antibody construct using A280 measurement and (ii) calculating the percent of correct and incorrect multispecific antibody construct module species produced using liquid chromatography-mass spectrometry (“LC-MS”), wherein steps (f)(i) and (f)(ii) can be performed simultaneously or in any order; and
(g) comparing the expression levels of (f)(i) and the percentage of correct and incorrect multispecific antibody construct module species of (f)(ii) for each multispecific antibody construct in order to identify the optimal pairing of three heavy chain CDRs and three light chain CDRs that specifically bind a first antigen with three heavy chain CDRs and three fight chain CDR-s that specifically bind a second antigen.

20. The method according to claim 19, wherein the multispecific antibody construct module is selected from the group consisting of Fab/Fab hetero Fc, scFab/scFab hetero Fc, Fab/scFv hetero Fc, Fab/Fab-scFv hetero Fc, Fab/scFv-Fab hetero Fc, Fab/Fab hetero Fc-scFv, IgG-Fab, scFab-Fc-Fab, IgG-scFv, scFv-IgG, and Fab-scFv-Fc.

21. A method for selecting a multispecific antibody construct, the method comprising:
(a) obtaining a first antibody Fab fragment or scFv, wherein each Fab fragment or scFv comprises three heavy chain CDRs and three light chain CDRs that specifically bind a first antigen;
(b) obtaining a second antibody Fab fragment or scFv, wherein each Fab fragment or scFv comprises three heavy chain CDRs and three light chain CDRs that specifically bind a second antigen;
(c) cloning into a vector:
(i) the CDRs of the three heavy chain CDRs that specifically bind the first antigen,
(ii) the CDRs of the three heavy chain CDRs that specifically bind the second antigen, and
(iii) three light chain CDRs selected from the group consisting of
(a) the CDRs of the three light chain CDRs that specifically bind the first antigen,
(b) the CDRs of the three light chain CDRs that specifically bind the second antigen, and
(c) CDRs of three light chain CDRs that do not specifically either the first antigen or the second antigen:
wherein the vector(s) encode(s) for more than one type of multispecific antibody construct module and a plurality of vectors are generated that encode for a plurality of multispecific antibody constructs of different modules comprising the heavy chain CDRs that bind to each antigen and the light chain CDRs of (c)(iii);
(d) expressing each multispecific antibody construct of the different modules in a mammalian host cell;
(e) purifying each multispecific antibody construct;
(f)(i) measuring the expression levels of each multispecific antibody construct and (ii) measuring the binding affinity of each multispecific antibody construct to the first antigen and the second antigen, wherein steps (f)(i) and (f)(ii) can be performed simultaneously or in any order; and
(g) comparing the expression levels of (f)(i) and the binding affinities of (f)(ii) for each multispecific antibody construct in order to identify the optimal module for pairing of the three heavy chain CDRs that specifically bind a first antigen and die light chain CDRs of (c)(iii) with the three heavy chain CDRs that specifically bind a second antigen and the same light chain CDRs of (c)(iii).

22. The method according to claim 21, wherein the multispecific antibody construct modules include at least two of the modules selected from the group consisting of Fab/Fab hetero Fc, scFab/scFab hetero Fc, Fab/scFv hetero Fc, Fab/Fab-scFv hetero Fc, Fab/scFv-Fab hetero Fc, Fab/Fab hetero Fe-scFv, IgG-Fab, scFab-Fc-Fab, IgG-scFv, scFv-IgG, and Fab-scFv-Fc.

23. The method according to claim 21, wherein the mammalian host cell is selected from the group consisting of Chinese hamster ovary (“CHO”) cells, monkey kidney CV1 line transformed by SV40 (“COS-7”); human embryonic kidney line HEK293-6E (“HEK293-6E”); human embryonic kidney line 293 (“HEK293”) baby hamster kidney cells (“BHK”); mouse sertoli cells (“TM4”); monkey kidney cells (“CV1”); African green monkey kidney cells (“VERO-76”); human cervical carcinoma cells (“HELA”); canine kidney cells (“MDCK”); buffalo rat liver cells (“BRL”): human lung cells (“W138”); human hepatoma cells (“Hep G2”): mouse mammary tumor (“MMT”); TRI cells: MRC 5 cells: and FS4 cells.

24. The method according to claim 21, wherein the expression levels are determined by a method selected from the group consisting of A280 measurement SDS-PAGE, microchip capillary electrophoresis (MCE), Bradford assay, and bicinchoninic acid (BCA) assay.

25. The method according to claim 21, wherein binding affinity of each multispecific antibody construct to the first antigen and the second antigen is measured using Octet, Forte Bio, Carterra LSA, SPR and Flow cytometry.

26. The method according to claim 21, wherein each multispecific antibody construct is purified by Protein A, Lambda and Kappa resins, and affinity tag purification.

27. A method for selecting a multispecific antibody construct, the method comprising:
(a) obtaining a first antibody Fab fragment or scFv, wherein each Fab fragment or scFv comprises three heavy chain CDRs and three light chain CDRs that specifically bind a first antigen:
(b) obtaining a second antibody Fab fragment or scFv, wherein each Fab fragment or scFv comprises three heavy chain CDRs and three light chain CDRs that specifically bind a second antigen;
(c) cloning into a vector:
(i) the CDRs of the three heavy chain CDRs that specifically bind the first antigen,
(ii) the CDRs of the three heavy chain CDRs that specifically bind the second antigen, and
(iii) three light chain CDRs selected from the group consisting of
(a) the CDRs of the three light chain CDRs that specifically bind the first antigen,
(b) the CDRs of the three light chain CDRs that specifically bind the second antigen, and
(c) CDRs of three light chain CDRs that do not specifically either the first antigen or the second antigen:
wherein the vector(s) encode(s) for more than one type of multispecific antibody construct module and a plurality of vectors are generated that encode for a plurality of multispecific antibody constructs of different modules comprising the heavy chain CDRs that bind to each antigen and the light chain CDRs of (c)(iii);
(d) expressing each multispecific antibody construct in a mammalian host cell, wherein the mammalian host cell is selected from the group consisting of HEK293-6E cells and CHO cells;
(e) purifying each multispecific antibody construct using Protein A chromatography;
(f)(i) measuring the expression levels of each multispecific antibody construct using A280 measurement and (ii) measuring the binding affinity of each multispecific antibody construct to the first antigen and the second antigen using Octet,
(g) comparing the expression levels of (f)(i) and the binding affinities of (f)(ii) for each multispecific antibody construct in order to identify the optimal module for pairing of the three heavy chain CDRs that specifically bind a first antigen and the light chain CDRs of (c)(iii) with the three heavy chain CDRs that specifically bind a second antigen and the same light chain CDRs of (c)(iii).

28. The method according to claim 27, wherein the multispecific antibody construct modules include at least two of the modules selected from the group consisting of Fab/Fab hetero Fc, scFab/scFab hetero Fc, Fab/scFv hetero Fc, Fab/Fab-scFv hetero Fc, Fab/scFv-Fab hetero Fc, Fab/Fab hetero Fc-scFv, IgG-Fab, scFab-Fc-Fab, IgG-scFv, scFv-IgG, and Fab-scFv-Fc.

29. A method for selecting a multispecific antibody construct, the method comprising:
(a) obtaining a first antibody Fah fragment or scFv, wherein each Fab fragment or scFv comprises three heavy chain CDRs and three light chain CDRs that specifically bind a first antigen;
(b) obtaining a second antibody Fab fragment or scFv, wherein each Fab fragment or scFv comprises three heavy chain CDRs and three light chain CDRs that specifically bind a second antigen;
(c) cloning the CDRs of the first antibody Fab fragment or scFv and the second antibody Fab fragment or scFv into a vector(s),
wherein the vector(s) encode(s) for more than one type of multispecific antibody construct module and a plurality of vectors are generated that encode for a plurality of multispecific antibody constructs of different modules comprising the CDRs that bind to each antigen:
(d) expressing each multispecific antibody construct in a mammalian host cell;
(e) purifying each multispecific antibody construct;
(f)(i) measuring the expression levels of each multispecific antibody construct and (ii) calculating the percent of correct and incorrect multispecific antibody construct module species produced, wherein steps (f)(i) and (f)(ii) can be performed simultaneously or in any order; and
(g) comparing the expression levels of (f)(i) and the percentage of correct and incorrect multispecific antibody construct module species of (f)(ii) for each multispecific antibody construct in order to identify the optimal multispecific antibody construct module for pairing of the three heavy chain CDRs and three light chain CDRs that specifically bind a first antigen with the three heavy chain CDRs and three light chain CDRs that specifically bind a second antigen.

30. The method according to claim 29, wherein the multispecific antibody construct modules include at least two of the modules selected from the group consisting of Fab/Fab hetero Fc, scFab/scFab hetero Fc, Fab/scFv hetero Fc, Fab/Fab-scFv hetero Fc, Fab/scFv-Fab hetero Fc, Fab/Fab hetero Fc-scFv, IgG-Fab, scFab-Fc-Fab, IgG-scFv, scFv-IgG, and Fab-scFv-Fc.

31. The method according to claim 29, wherein the mammalian host cell is selected from the group consisting of Chinese hamster ovary (“CHO”) cells, monkey kidney CV1 line transformed by SV40 (“COS-7”); human embryonic kidney line HEK293-6E (“HEK293-6E”); human embryonic kidney line 293 (“HEK293”); baby hamster kidney cells (“BHK”); mouse sertoli cells (“TM4”); monkey kidney cells (“CV1”); African green monkey kidney cells (“VERO-76”); human cervical carcinoma cells (“HELA”); canine kidney cells (“MDCK”); buffalo rat liver cells (“BRL”): human lung cells (“W138”); human hepatoma cells (“Hep G2”): mouse mammary tumor (“MMT”); TRI cells; MRC 5 cells: and FS4 cells.

32. The method according to claim 29, wherein the expression levels are determined by a method selected from the group consisting of A280 measurement SDS-PAGE, microchip capillary electrophoresis (MCE), Bradford assay, and bicinchoninic acid (BCA) assay.

33. The method according to claim 29, wherein the percentage of correct and incorrect multispecific antibody construct modality species is determined by a method selected from the group consisting of liquid chromatography-mass spectrometry (“LC-MS”), Caliper, HPLC SEC, SDS-PACE, and microchip capillary electrophoresis (“MCE”).

34. The method according to claim 29, wherein each multispecific antibody construct is purified by Protein A, Lambda and Kappa resins, and affinity tag purification.

35. A method for selecting a multispecific antibody construct, the method comprising:
(a) obtaining a first antibody Fab fragment or scFv, wherein each Fab fragment or scFv comprises three heavy chain CDRs and three light chain CDRs that specifically bind a first antigen;
(b) obtaining a second antibody Fab fragment or scFv, wherein each Fab fragment or scFv comprises three heavy chain CDRs and three light chain CDRs that specifically bind a second antigen;
(c) cloning the CDRs of the first antibody Fab fragment or scFv and the second antibody Fab fragment or scFv into a vector(s),
wherein the vector(s) encode(s) for more than one type of multispecific antibody construct module and a plurality of vectors are generated that encode for a plurality of multispecific antibody constructs of different modules comprising the CDRs that bind to each antigen:
(d) expressing each multispecific antibody construct in a mammalian host cell, wherein the mammalian host cell is selected from the group consisting of HEK293-6E cells and CHO cells;
(e) purifying each multispecific antibody construct using Protein A chromatography;
(f)(i) measuring the expression levels of each multispecific antibody construct using A280 and (ii) calculating the percent of correct and incorrect multispecific antibody construct module species produced using liquid chromatography-mass spectrometry (“LC-MS”),
wherein steps (f)(i) and (f)(ii) can be performed simultaneously or in any order; and
(g) comparing the expression levels of (f)(i) and the percentage of correct and incorrect multispecific antibody construct module species of (f)(ii) for each multispecific antibody construct in order to identify the optimal multispecific antibody construct module for pairing of the three heavy chain CDRs and three light chain CDRs that specifically bind a first antigen with the three heavy chain CDRs and three light chain CDRs that specifically bind a second antigen.

36. The method according to claim 35, wherein the multispecific antibody construct modules include at least two of the modules selected from the group consisting of Fab/Fab hetero Fc, scFab/scFab hetero Fc, Fab/scFv hetero Fc, Fab/Fab-scFv hetero Fc, Fab/scFv-Fab hetero Fc, Fab/Fab hetero Fc-scFv, IgG-Fab, scFab-Fc-Fab, IgG-scFv, scFv-IgG, and Fab-scFv-Fc.
